SEQUENCE LISTING (298 sequences)

SEQ ID NO: 1 - protein, 245 aa, Bos taurus
CGVPAIQPVLSGLSRIVNGEEAVPGSWPWQVSLQDKTGFHFCGGSLINENWVVTAAHCGVTTSDVVVAGEFDQGSSSEKIQKLKIAKVFKNSKYNSLTINNDITLLKLSTAASFSQTVSAVCLPSASDDFAAGTTCVTTGWGLTRYTNANTPDRLQQASLPLLSNTNCKKYWGTKIKDAMICAGASGVSSCMGDSGGPLVCKKNGAWTLVGIVSWGSSTCSTSTPGVYARVTALVNWVQQTLAAN

SEQ ID NO: 2 - protein, 245 aa, Sus scrofa
CGVPAIDPMLSGLSRIVNGEEAVPGSWPWQVSLQTSSGFHFCGGSLISEAWVVTAAHCEVRKSHLVVAGVSDLSSDEEAVQVLRIAEVVEHPLWNLHALRNDIALLKLATPARLSGAVSPVCLPSTNTSFPTGSLCATTGWGKTRHNALKTPDKLQQAALPIVSSADCKSYWGSKVTDVMICAGASGVSTCMGDSGGPLVCQKDGAWTLAGIVSWGSGRCLPFSPGVYARVTKFVPWILEVLKAN

SEQ ID NO: 3 - protein, 245 aa, Sus scrofa
CGVPAIDPMLSGLSRIVNGEEAVPGSWPWQVSLQTSSGFHFCGGSLISEAWVVTAAHCEVRKSHLVVAGVSDLSSDEEAVQVLRIAEVVEHPLWNLHALRNDIALLKLATPARLSGAVSPVCLPSTQTSFPTGSLCATTGWGKTRHNALKTPDKLQQAALPIVSSADCKSYWGSKVTDVMICAGASGVSTCMGDSGGPLVCQKDGAWTLAGIVSWGSGRCLPFSPGVYARVTKFVPWILEVLKAN

SEQ ID NO: 4 - protein, 245 aa, Gadus morhua
CGRPAISPVITGYSRIVNGEEAVPHSWSWQVSLQDQTGFHFCGGSLINENWVVTAAHCNVKNYHRVVLGEHDRSSNSEGVQVMTVGQVFKHPRYNGFTINNDILLVKLATPATLNMRVSPVCLAETDDVFEGGMKCVTSGWGLTRYNAADTPALLQQAALPLLTNEQCKKFWGNKISDLMICAGAAGASSCMGDSGGPLVCQKAGSWTLVGIVSWGSGTCTPTMPGVYARVTELRAWVDQTIAAN

SEQ ID NO: 5 - protein, 251 aa, Gadus morhua
AYGCGSPAIQPQVTGYARIVNGEEAVPHSWPWQVSLQQSNGFHFCGGSLINENWVVTAAHCNVRTYHRVIVGEHDKSSAITSEKVQILKPSKVFTHPEWNSRTINNDISLIKLASPAVLGTTVSPVCLGESSEVFAPGMKCVTSGWGLTRYNAPGTPNKLQQAALPLMSNEQCKRSWGSNMISDVMICAGAAGATSCMGDSGGPLVCQKDNVWTLVGIVSWGSSRCSTSTPAVYARVTELRGWVDQILAAN

SEQ ID NO: 6 - protein, 245 aa, Danio rerio
CGVPAIPPVVTGYARIVNGEEARPHSWPWQVSLQDSTGFHFCGGSLINENWVVTAAHCNVRTSHRVILGEHDRSSNAEAIQTIAVGKSIKHPNYNSFTINNDILLIKLATPAKINTHVSPVCLAETNDNFPGGMKCVTSGWGLTRYNAPDTPALLQQAALPLLTNDDCKRYWGTQITDLMICAGASGVSSCMGDSGGPLVCENNRVWTLVGIVSWGSSTCSTSTPAVYARVTKLRAWVDQTIASN

SEQ ID NO: 7 - protein, 219 aa, Beauveria bassiana
IAGGQDAELGWFPYTVSLRNRGRHNCGGIIIDQSSILTAAHCIVGLSADRVSIVAGAVKLDSGGTTYQVSAIHKHPGYNSLTITNDIAVLKLAKPLSYTPYIRPIALATQDPPAGSGTLGSGWGVSRSSGPVSNTLQWISLQSLDHKICQEKMGVTVSQICTTSANKGGMCRGDSGGPLTDERITLVGVYSWGRGCGDGYPDVYASVAYHREWIRSYMG

SEQ ID NO: 8 - protein, 238 aa, Daboia siamensis
QKSSELVVGGDECNINEHRSLVFLYNNSFGCSGTLINQQWVLSAVHCDMENVRIYLGVHNLRLRNNAEIRLPEERFFCLSSKSYTKWDKDIMLIKLDRPVKTSTYIAPLSLPSSPPRVGSVCRIMGWGAITSPQETFPGVTHCANINILPYSVCRAAYKGLPAQSRTLCGGILEGGIGSCMGDSGGPLICNGEMHGIVAWGDDTCAQPHKPVHYTKVYDYTDWIQSIIAGNTAATCPP

SEQ ID NO: 9 - protein, 239 aa, Macrovipera lebetinus
QKSSELVVGGDECNINEHRSLVFLYNSSFGCGGTLINQQWVLSAAHCDMENVQIYLGLHNLRLRNQDEQIRVAEEKFFCLSNKSYTKWDKDIMLIRLNSSVTYNTHIAPLSLPSSPPRVGSVCRIMGWGAITSPNETFPNVPHCANINILRYSVCRAAYRGLPAQSRTLCAGILQGGIGSCMGDSGGPLICNGEIQGIVSWGDDICAQPHKPVHYTKVFDYSDWIQSIIAGNTTATCPL

SEQ ID NO: 10 - protein, 239 aa, Macrovipera lebetinus
QKSSELVVGGDECNINEHRSLVFLYQSSFGCGGTLINQQWVLSAAHCDMENVQIYLGLHNLRLRNQDEQIRVAEEKFFCLSQKSYTKWDKDIMLIRLQSSVTYNTHIAPLSLPSSPPRVGSVCRIMGWGAITSPQETFPNVPHCANINILRYSVCRAAYRGLPAQSRTLCAGILQGGIGSCMGDSGGPLICNGEIQGIVSWGDDICAQPHKPVHYTKVFDYSDWIQSIIAGQTTATCPL

SEQ ID NO: 11 - protein, 239 aa, Macrovipera lebetinus
QKSSELVVGGDECNINEHRSLVFLYQSSFGCGGTLINQQWVLSAAHCDMENVQIYLGLHNLRLRNQDEQIRVAEEKFFCLSQKSYTKWDKDIMLIRLQSSVTYNTHIAPLSLPSSPPRVGSVCRIMGWGAITSPQETFPNVPHCANINILRYSVCRAAYRGLPAQSRTLCAGILQGGIGSCMGDSGGPLICNGEIQGIVSWGDDICAQPHKPVHYTKVFDYSDWIQSIIAGNTAATCPL

SEQ ID NO: 12 - protein, 240 aa, Macrovipera lebetinus
EKSSELVIGGRPCNINQHRSLALLYNSSGFLCGGTLINQQWVLSAAHCDMENMQIYLGLHNFSLPNMDQKRRVAEEKFFCLSNKSYTKWDKDIMLIKLNRRVKTSTHIAPLSLPSNPPRLRSVCRIMGWGSITSPRETLPYVPHCANIMILRYWVCRAIYGSLPAKSRTLCAGVPRRRIGSCLGDSGGPLICNGQIQGIASWGSDPCVNHGAPGVYTKVFDYNDWIQSIIAGNTAATCPP

SEQ ID NO: 13 - protein, 240 aa, Macrovipera lebetinus
EKSSELVIGGRPCNINQHRSLALLYQSSGFLCGGTLINQQWVLSAAHCDMENMQIYLGLHQFSLPNMDQKRRVAEEKFFCLSQKSYTKWDKDIMLIKLNRRVKTSTHIAPLSLPSNPPRLRSVCRIMGWGSITSPRETLPYVPHCANIMILRYWVCRAIYGSLPAKSRTLCAGVPRRRIGSCLGDSGGPLICNGQIQGIASWGSDPCVNHGAPGVYTKVFDYNDWIQSIIAGNTAATCPP

SEQ ID NO: 14 - protein, 245 aa, Capra hircus
CGVPAIQPVLSGLARIVNGEDAVPGSWPWQVSLQDSTSFHFCGGSLISEDWVVTAAHCGVTTSDVVVAGEFDQGSQTENTQVLKIGKVFKNPKFSILTVRNDITLLKLATPAQFSETVSAVCLPSTDEDFPAGMLCATTGWGKTKYNALKTPDKLQQATLPIVSNTDCKKYWGSRVTDVMICAGASGVSSCMGDSGGPLVCQKDGVWTLAGIVSWGSSTCSTSTPAVYARVTALMPWVQETLAAN

SEQ ID NO: 15 - protein, 245 aa, Rattus norvegicus
CGVPTIQPVLTGLSRIVNGEDAIPGSWPWQVSLQDKTGFHFCGGSLISEDWVVTAAHCGVKTSDVVVAGEFDQGSDEENIQVLKIAQVFKNPKFNMFTVRNDITLLKLATPAQFSETVSAVCLPNVDDDFPPGTVCATTGWGKTKYNALKTPEKLQQAALPIVSEADCKKSWGSKITDVMTCAGASGVSSCMGDSGGPLVCQKDGVWTLAGIVSWGSGVCSTSTPAVYSRVTALMPWVQQILEAN

SEQ ID NO: 16 - protein, 245 aa, Oryctolagus cuniculus
CGVPAIQPVLTGLSRIVNGEDAVPGSWPWQVSLQDKTGFHFCGGSLISENWVVTAAHCGVSTSHLVVAGEFDQGSSQEKVQVLKIAKVFKNPKFNIFTIRNDITLLKLATPASFSQTVSAVCLPNADDDFPAGTLCATTGWGKIRYNANTTPEKLQQAALPLLSNADCQKYWGTKITSVMICAGANGVSSCIGDSGGPLVCQKNGAWTLVGIVSWGSNTCSTSTPAVYTRVTELIPWVQEILAAN

SEQ ID NO: 17 - protein, 245 aa, Balaenoptera acutorostrata
  source 1..245: note = subspecies scammoni
CGVPAIDPVLSGLSRIVNGEDAVPGSWPWQVSLQDSTGFHFCGGSLISEDWVVTAAHCGVKTSDLVVAGEFDQGSDAENIQVLKIAKVFKNPKFNMLTVRNDITLLKLATPARFSETVSPVCLPSANDDFPAGMLCVTTGWGKTRYNAPKTPDKLQQATLPILSNANCRKYWGNKITDVMICAGASGVSSCMGDSGGPLVCQKDGAWTLVGIVSWGSGTCSTSTPAVYARVTALIPWVQKILAAN

SEQ ID NO: 18 - protein, 245 aa, Gallus gallus
CGVPAITPVIRGYNRIVNGEPAVPGSWPWQVSLQRYGNFHFCGGSLISEQWVVTAAHCGVRTTDTVVLGEYDQETASSDVQRLGIAKVFRNPSYSSLTIRNDITLIKLATPAQLNARVAPVCLPQATDDFPGGLTCVTTGWGLLNSNDENTPAILQQAALPLLTNAQCKQYWGFRIYDVMVCAGADGSSSCMGDSGGPLVCQKDGAWTLVGIVSWGSSTCSTSTPGVYARVTKLREWIDSVLAAN

SEQ ID NO: 19 - protein, 245 aa, Crocodylus porosus
CGTPAIQPIISGYARIVNGEEAIPGSWPWQVSLQDNTGFHFCGGSLISEDWVVTAAHCGVKTTDRVVLGAFDLGFLGEDVQVLKIAQVFKNRNFNMLTLRDDITLLKLATPAKMTARVSPVCLPEATEYFSGGITCVTTGWGLTNPNAQKTPDKLQQVALPLLTNVQCKSYWSYRIKDCMVCAGAKGASSCMGDSGGPLVCQKDGIWTLVGIVSWGSNTCSPSSPGVYTRVSKLRPWINEIIAAN

SEQ ID NO: 20 - protein, 243 aa, Bombus vosnesenskii
FPEGQIVGGKDAPVGKFPHQVSLRQSGSHFCGGSIIDSRHILTAAHCVQGLNNLKSITVHAGTNQLNTNGQSYGVEKAVAHKGFNSNTLLNDIAIIRVNQNIAFNNLVKSIKLASGSNTYEGSNCILSGWGTTRLGGKSPNNLQYINLLVESQSKCKQAHWRVQSSHICTYTKVGEGACHGDSGGPLIHDDVQIGVVSFGQPCAVGKPDVYTRVSSFIPWINSQKSYLMNETEEQPEDAIYIA

SEQ ID NO: 21 - protein, 245 aa, Apis cerana
YGFPNNQIVGGKDAPVGKFPYQVSLRKNKNHFCGGSIIDSRTILTAAHCVEGLSNLNGITVQAGTNQLNSGGVSYVPEKVVAHSGFNSLSLVNDIALIRVNQAISFNNLVQPIKLASGTNTYEGSSCVLSGWGTVKLNGNVPNNLQYINLLIEKQSECKKVHWRVQSSHICTFTKYGEGACHGDSGGPLVVNGVQVGIVSFGQPCAVGKPDVYTRVSSFSSWIQQHKSFLMEEKSEAPADGVYIS

SEQ ID NO: 22 - protein, 229 aa, Solenopsis invicta
YGVPESKIVGGKDAPVGKYPYQVSLRLSGSHRCGASILDNNNVLTAAHCVDGLSNLNRLKVHVGTNYLSESGDVYDVEDAVVNKNYDDFLLRNDVALVHLTNPIKFNDLVQPIKLSTNDEDLESNPCTLTGWGSTRLGGNTPNALQEIELIVHPQKQCERDQWRVIDSHICTLTKRGEGACHGDSGGPLVANGAQIGIVSFGSPCALGEPDVYTRVSSFVSWINANLKK

SEQ ID NO: 23 - protein, 246 aa, Danaus plexippus
FILPQFYDSPSPRIVGGENAPVGSAPHQASLRSIFGSHFCGGSIISKRWILTAAHCTVGQSTFTIKVVVGTNSLTSGGEKYSVEKIIVHEDYDGGEIINDVSVVKVSTDIEFTDLVQPIQLPKTDTAEGAVLTLTGWGRTSYPGSLPDRLQVISLIALSVNQCQQIYKGIVSSNVYDTQICSLTKSGEGACHGDSGGPLVEGDKVVGIVSWGVPCARGYPDVYTRVYSFKDWILSKTNMTDESIIN

SEQ ID NO: 24 - protein, 240 aa, Photinus pyralis
AVSPFNNLFGTVPKDEFDWKIVGGSDAPEGAYPYQVSLRRFGSHSCGGSIVDATTVMTAAHCVTGAKPSALRVVVGSNKLNSGGVSYEIESIRDHKGFDSWRLTNDIAILKLKTPITFTDKVKTVDLETEYLGAEVDCVLSGWGRLNYPGSLPNNLQHIGLKTITSEDCAKLLNHSSVDRTHICTLTKSGEGACHGDSGGPLVANKQIGIVSFGRPCAKGYPDAFTRVSAFTDWIEKNRY

SEQ ID NO: 25 - protein, 237 aa, Aedes aegypti
TLSPEFLEWEGRIVGGQNAGSGQFPWQVSLRSSGNAHFCGGSIINNRWVLSAAHCTVGRTLANTRVVVGTHLLNSGGVAHNSARIVNHPSYNANSLTNDISLVQTASIIAFNNLAQPIGLGATFINTASGALASGWGQLGANVGIPNNLQWLSTSIITLADCRNRHSVVNRGRVHDNTICTLNPSGQGMCMGDSGGPLVHGGNQQGVVSWGIPCGLGSPDVFARVSSHRTWILNNAV

SEQ ID NO: 26 - protein, 245 aa, Tenebrio molitor
LPLNEKITPFNATSGRIINGSPASKGQFPWQAAVIFTTSSGQSFCGGSIISNQWILTAAHCARGATSFTIYLGTNTLTPDSGSTTVTTTRAVIHPSYNPSTLANDVALIQLTSPITFNTYVQPITLASSTLGSGVSVTVSGWGKTSDSASGVTNTLNYVTLSSITNTQCSNYYGSLGSGVVCATGASKSTCNGDSGGPLVTGSGSNAVHVGVVSFVSSRGCESGYPSGYSRTAYYRDWIRTTTGV

SEQ ID NO: 27 - protein, 238 aa, Tenebrio molitor
SPISERIVNGTDAREGEIPYIVSLRLQDRHTCGGSILDSRHILTAAHCVQYDVIYYSVQYGVTRISSALNAPNVIEVSKITVHEGYVPGNGYLNDVAVVELKSDIPFGVHVQPVKLPSLAAATPENAPATLSGWGYPYTGGAVMEDLQTVDIVVFSDADCERIHAGTGPTYSNYHVCAGIPEGGKGQCSGDSGGPLVVDGVQVGIVSWSVKPCTVQGFPGVFIKVSTQLGWIQRQVAA

SEQ ID NO: 28 - protein, 241 aa, Tenebrio molitor
TPVNVDSNIDWRVVGGSTATPHQFPFIVSLRTPYDSHNCGGSIIAKNYVITAAHCVSGYAPSYYTVVAGTNQLNATNPLRLKVAQIIVHPEYSSSLILNDVALLRLETPVEESEEVQIVGLETEYVDTVRDCVLVGWGRTSYPGSIPNDLQFLNERTYPNDECVNRWASAHAVYSSQICTLTKVGEGACHGDSGGPLVVVKDDKFSLIALVSWGSPCARGMPDVYTRVASFHEFITDNIKN

SEQ ID NO: 29 - protein, 252 aa, Diatraea saccharalis
EQIREREERSLSQRIVGGSSAEEGQFPYQAGLLTDMIGLQGNGVCGGSLVSANRVVTAAHCWFDGMHQAWRVTVILGSTRLFSGGQRLATSAVVTHPGWQPPLIRHDVAIIYLPSNAVMSNTVAPIALPSGDLLNQNFVGELATASGFGLTSDNANITTNQFLSHVNLNVISNNICRFSFPLLIQEDHICTSGLGGVGTCRGDSGGPLVIFRNNSPILIGITSFGSPLGCESIMPAAYARVTEYVDFINQHL

SEQ ID NO: 30 - protein, 234 aa, Apis mellifera
ENLEKIVGGTNASPGQFPYQVSLRKSGRHFCGGTLITERHIVTAAHCIHGIVSAPYNDFTVVTGTISNINGGQSYCVAKATVNPDFKPSSSESYRNDIAIVTLADTVKSNTYQKPISPASSDPPVGATLIMSGWGRTSTNGNLPEILQTTNVYLMSNEECQKRIPNYHIYNGQLCTFKRKGVGICMGDSGGPLVYNGELIGIASWVIPCAQGYPDAYTRVTQYRNFINQIVSNI

SEQ ID NO: 31 - protein, 238 aa, Apis mellifera
LSWNYNPKIINGEDAKEGEIPYQVSLQNKFSSFHFCGGSILNENYVITAAHCVHGKFSEDIKVVAGTINLANPRYENDVNEIIVHEKYNVSDSWKNDIALLKVKTSFTLSNTISSVPLPSPNDIIKPNDLATVSGWGRLRQGGPTTIYLQRVNILIANQEYCELTYKKINYTVYESQICAYYPTSEKGSCNGDSGGPLTVNGKLVGLVSWAMGCALIDYPTVYTRVESYLDWIKENAI

SEQ ID NO: 32 - protein, 245 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVVGDLQVGIVSFGQPCAVGKPDVYTRVSSFTSWINEHKSFLMEERGEAPADGFYIS

SEQ ID NO: 33 - protein, 245 aa, Apis mellifera; identical to SEQ ID NO: 32

SEQ ID NO: 34 - protein, 246 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGSCHGDSGGPLVCGDLQVGIVSFGQDICAVGKPVVYTRVSSFTSWINEHKSFLMEERGEAPADGFYIS

SEQ ID NO: 35 - protein, 245 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDAILSGWGTTKLNGNVPNNLQWIKLKIETEQKCKQAHWRVQSSHICTFTKSGEGACHGDSGGPLVVGDLQVGIVSFGQPCAVGKPDVYTRVSSFTSWINEHKSFLMEERGEAPADGFYIS

SEQ ID NO: 36 - protein, 245 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDACLSGWGTTKLNGNVPNNLQWIKLKIETEQKCKQAHWRVQSSHICTFTKSGEGACHGDSGGPLCVGDLQVGIVSFGQPCAVGKPDVYTRVSSFTSWINEHKSFLMEERGEAPADGFYIS

SEQ ID NO: 37 - protein, 245 aa, Apis mellifera
YGVPESKIVGGKNALSGQFPYQVSLRKNGSHFCGGSIIDSRTILTAAHCVEGLSNLNGITVVAGTNQLNSGGVSYVPEKVVAHPSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVCGDLQVGIVSFGQPCAVGKPDVYTRVSSFTSWINEHKSFLMEERGEAPADGFYIS

SEQ ID NO: 38 - protein, 245 aa, Apis mellifera; identical to SEQ ID NO: 37

SEQ ID NO: 39 - protein, 245 aa, Apis mellifera
YGVPESKIVGGKNALSGQFPYQVSLRKQGSHFCGGSIIDSRTILTAAHCVEGLSNLNGITVVAGTNQLNSGGVSYVPEKVVAHPSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVCGDLQVGIVSFGQPCAVGKPDVYTRVSSFTSWINEHKSFLMEERGEAPADGFYIS

SEQ ID NO: 40 - protein, 245 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGPCHGDSGGPLVVGDLQVGIVSFGQPCAVGKPDVYTRVSSFTSWINEHKSFLMEERGEAPADGFYIS

SEQ ID NO: 41 - protein, 245 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGVCHGDSGGPLVVGDLQVGIVSFGQPCAVGKPDVYTRVSSFTSWINEHKSFLMEERGEAPADGFYIS

SEQ ID NO: 42 - protein, 246 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVVGGPLQVGIVSFGQPCAVGKPDVYTRVSSFTSWINEHKSFLMEERGEAPADGFYIS

SEQ ID NO: 43 - protein, 245 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVVGDLQVGIVSFGQPCAVGKPGVYTRVSSFTSWINEHKSFLMEERGEAPADGFYIS

SEQ ID NO: 44 - protein, 245 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVVGDLQVGIVSFGQPCAVGKPHVYTRVSSFTSWINEHKSFLMEERGEAPADGFYIS

SEQ ID NO: 45 - protein, 245 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIITEQKCKQAHWRVQSSHICTFTKSGEGACHGDSGGPLVVGDLQVGIVSFGQPCAVGKPDVYTRVSSFTSWINEHKSFLMEERGEAPADGFYIS

SEQ ID NO: 46 - protein, 245 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTLTKSGEGACHGDSGGPLVVGDLQVGIVSFGQPCAVGKPDVYTRVSSFTSWINEHKSFLMEERGEAPADGFYIS

SEQ ID NO: 47 - protein, 245 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVVGDLQVGIVSWGQPCAVGKPDVYTRVSSFTSWINEHKSFLMEERGEAPADGFYIS

SEQ ID NO: 48 - protein, 230 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVVGDLQVGIVSFGQPCAVGKPDVYTRVSSFTSWINEHKSNI

SEQ ID NO: 49 - protein, 245 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVVGDLQVGIVSFGQPCAVGKPDVYTRVSSFTSWINEHKSFLMEERGEAPADGARIS

SEQ ID NO: 50 - protein, 245 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSIEGACHGDSGGPLVVGDLQVGIVSFGQPCAVGKPDVYTRVSSFTSWINEHKSFLMEERGEAPADGFYIS

SEQ ID NO: 51 - protein, 246 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGESSCHGDSGGPLVCGDLQVGIVSFGQSTCAVGKPVVYTRVSSFTSWINEHKSFLMEERGEAPADGFYIS

SEQ ID NO: 52 - protein, 245 aa, Apis mellifera
YGVPESYIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVVGDLQVGIVSFGQPCAVGKPDVYTRVSSFTSWINEHKSFLMEERGEAPADGFYIS

SEQ ID NO: 53 - protein, 245 aa, Apis mellifera
YGVPESYIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVVGDLQVGIVSFGQPCAVGKPDVYTRVSSFTSWINEHKSFLMEERGEAPADGARIS

SEQ ID NO: 54 - protein, 230 aa, Apis mellifera
YGVPESYIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVVGDLQVGIVSFGQPCAVGKPDVYTRVSSFTSWINEHKSAN

SEQ ID NO: 55 - protein, 245 aa, Apis mellifera
YGVPESYIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVCGDLQVGIVSFGQPCAVGKPDVYTRVSSFTSWINEHKSFLMEERGEAPADGFYIS

SEQ ID NO: 56 - protein, 228 aa, Apis mellifera
YGVPESYIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVCGDLQVGIVSFGQPCAVGKPDVYTRVSSFTSWINEHKS

SEQ ID NO: 57 - protein, 245 aa, Apis mellifera
YGVPESKIVGGKNALSGQFPYQVSLRKQKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVVGDLQVGIVSFGQPCAVGKPDVYTRVSSFTSWINEHKSFLMEERGEAPADGFYIS

SEQ ID NO: 58 - protein, 245 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVVGDLQVGIVSFGQACAVGKPDVYTRVSSFTSWINEHKSFLMEERGEAPADGFYIS

SEQ ID NO: 59 - protein, 245 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVVGDLQVGIVSFGQCPAVGKPDVYTRVSSFTSWINEHKSFLMEERGEAPADGFYIS

SEQ ID NO: 60 - protein, 245 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVCGDLQVGIVSFGQPCAVGKPDVYTRVSSFTSWINEHKSFLMEERGEAPADGFYIS

SEQ ID NO: 61 - protein, 228 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVCGDLQVGIVSFGQPCAVGKPDVYTRVSSFTSWINEHKS

SEQ ID NO: 62 - protein, 245 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVVGDLQVGIGSFGQPCAVGKPDVYTRVSSFTSWINEHKSFLMEERGEAPADGFYIS

SEQ ID NO: 63 - protein, 245 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVVGDLQVGIWSFGQPCAVGKPDVYTRVSSFTSWINEHKSFLMEERGEAPADGFYIS

SEQ ID NO: 64 - protein, 228 aa, Apis mellifera
YGVPESKIVGGKNALSGQFPYQVSLRKNKSHFCGGSIIDSRTILTAAHCVEGLSNLNGITVQAGTNQLNSGGVSYVPEKVVAHRSFNALSLVNDIALIRVNQDISFTNLIQPIKLASGSKTYEGSDCILSGWGTTKLNGNVPNNLQWIKLKIETEQKCKQAHWRVQSSHICTFTKSGEGACHGDSGGPLVVGDLQVGIVSFGQPCAVGKPDVYTRVSSFTSWINEHKS

SEQ ID NO: 65 - protein, 395 aa, Gammaproteobacteria bacterium
SVSPRIVGGTLSRENAWPFMTALMQKAAELNIAQESYTAFFLAGVPQRSFSGALVNCEQGFDVCSNAKGKICLIERGITFFSEKIANCRIGGGIAAVIYNNEVSSLSWRADNAKIPAVSISQNDGQILLNHLGEQIQYVPLDTIPTTSFCGGSYIGGKWVVTAAHCVSQVSADLIALNIGGQNLETDQDNIYYAKRIALHQRWNPETIENDVALIELTTEPQGITPIKIADESQLTQAIEDFTEVTAIGRGSKLATSSKLVTELERPDPLLYEVQLSVVSNRNCEKIFDEYEISNGADPTIVDTTTNIASGMLCAGNLAGGEGTCFGDSGGPLVLNQNGENYLIGLTSWGVGCAQPDLYGVYSRAPHYKDSIEAVLNDQYLTRPTDFPSLRPRKS

SEQ ID NO: 66 - protein, 647 aa, Pseudoalteromonas sp.
KQVTISPVKPKIVGGEEATEGVYPWMAAIVATAPSVATSLKVGENSIETSPFSFSPAGAVSGELASCELGDAECTDVEDKVCLIERGTVTFAEKANNCEAGGGVGVVIYNNVEGHFSGTLGEGFEGTIPVVGITQADGQALLDEVATAEKAGSTAAIEVSAEETLTQQSSCGASFLGDRWVLTAAHCVDSPNSINMKVNVGEFDLADGAEDAIEIKNLYLHPGYDTNTLHDDIALIELAESVDKPTIKIADLELTSQLATDNSVATSIGWGGRTGYLPGEGPTSDFPEILHEVDLQLYSSLQCRTIIAESRTRDTDFEFTEADVDVTEQMICAGTPAGGLSTCQGDSGGPLIVNTNDGWQQVGVVSWGLGCAAPGYPGVYTRAGAYQDWMDGITDGIAITQLHDFTTLPETDSRTFKLMVEQNSGEAASVTYEIEGSSTFTVEEGACNTLAAGETCELSATYTPVEAGAHEAFVVIKSDNAALKSSKTKLMGNALASASDLKDASGSTSEEVSLFTGGDVKWEENAEGGLQSGDIDDGQESILMAEVNGEGKLTFEWGVSSEENVEDPAEPWDALYLTVNGELHSYISGDVAFESVELELPEGKNLITWSYIKDGAVTELEDTGYVRNINFAKKEPIVIPQMPITEQ

SEQ ID NO: 67 - protein, 240 aa, Scytalidium lignicola
APGGNGFARRQARRQARAAGLKASPFRQVNAKEATVESNWGGAILIGSDFDTVSATANVPSASGGSSAAGTAWVGIDGDTCQTAILQTGFDWYGDGTYDAWYEWYPEVSDDFSGITISEGDSIQMSVTATSDTSGSATLEQLTTGQKVSKSFSQESSGSLCRTNAEFIIEDFEECNSQGSDCEFVPFASFSPAVEFTDCSVTSDGESVSLDDAQITQVIINNQDVTDCSVSGTTVSCSYV

SEQ ID NO: 68 - protein, 355 aa, Metarhizium anisopliae
APTQAASLHPQILEAMKRDLGLNAEQATVRVAREIHATDVIEQLRSSVAFAGAWIDADVLYIGITDQALADEVTAAGATPIVMTNSLSKLEKAKEDLDKIFIGRANTLETSSDTSSGIASYFVDVAANKLVIEALADSHGHAEQLAAQVGLTSEFEVRTVETMPTTMATVQGGDVYYINRSSRCSIGFAVTTGFVSAGHCGGSGASATTSSGEALGTFSGSVFPGSADMAYVRTVSGTVLRGYINGYGQGSFPVSGSSEAAVGASICRSGSTTQVHCGTIGAKGATVNYPQGAVSGLTRTSVCAEPGDSGGSFYSGSQAQGVTSGGSGDCSRGGTTYFQPVNRILQTYGLTLVTA

SEQ ID NO: 69 - protein, 337 aa, Thermobifida fusca
QELALKRDLGLSDAEVAELRAAEAEAVELEEELRDSLGSDFGGVYLDADTTEITVAVTDPAAVSRVDADDVTVDVVDFGETALNDFVASLNAIADTADPKVTGWYTDLESDAVVITTLRGGTPAAEELAERAGLDERAVRIVEEDEEPQSLAAIIGGNPYYFGNYRCSIGFSVRQGSQTGFATAGHCGSTGTRVSSPSGTVAGSYFPGRDMGWVRITSADTVTPLVNRYNGGTVTVTGSQEAATGSSVCRSGATTGWRCGTIQSKNQTVRYAEGTVTGLTRTTACAEGGDSGGPWLTGSQAQGVTSGGTGDCRSGGITFFQPINPLLSYFGLQLVTG

SEQ ID NO: 70 - protein, 423 aa, Streptomyces griseus
DPAATAAPPVSADSLSPGMLAALERDLGLDEDAARSRIANEYRAAAVAAGLEKSLGARYAGARVSGAKATLTVATTDASEAARITEAGARAEVVGHSLDRFEGVKKSLDKAALDKAPKNVPVWYVDVAANRVVVNAASPAAGQAFLKVAGVDRGLVTVARSAEQPRALADIRGGDAYYMNGSGRCSVGFSVTRGTQNGFATAGHCGRVGTTTNGVNQQAQGTFQGSTFPGRDIAWVATNANWTPRPLVNGYGRGDVTVAGSTASVVGASVCRSGSTTGWHCGTIQQLNTSVTYPEGTISGVTRTSVCAEPGDSGGSYISGSQAQGVTSGGSGNCSSGGTTYFQPINPLLQAYGLTLVTSGGGTPTDPPTTPPTDSPGGTWAVGTAYAAGATVTYGGATYRCLQAHTAQPGWTPADVPALWQRV

SEQ ID NO: 71 - protein, 360 aa, Streptomyces sp.
SSELPSAETAKADALVEQLPAGMVDAMERDLGVPAAEVGNQLVAEHEAAVLEESLSEDLSGYAGSWIVEGTSEHVVATTDRAEAAEITAAGATATVVEHSLAELEAVKDILDEAATANPEDAAPVWYVDVTTNEVVVLASDVPAAEAFVAASGADASTVRVERSDESPQPFYDLVGGDAYYIGNGRCSIGFSVRQGSTPGFVTAGHCGSVGNATTGFNRVSQGTFRGSWFPGRDMAWVAVNSNWTPTSLVRNSGSGVRVTGSTQATVGSSICRSGSTTGWRCGTIQQHNTSVTYPQGTITGVTRTSACAQPGDSGGSFISGTQAQGVTSGGSGNCSIGGTTFHQPVNPILSQYGLTLVRS

SEQ ID NO: 72 - protein, 360 aa, Streptomyces sp.
SSELPSAETAKADALVEQLPAGMVDAMERDLGVPAAEVGNQLVAEHEAAVLEESLSEDLSGYAGSWIVEGTSEHVVATTDRAEAAEITAAGATATVVEHSLAELEAVKDILDEAATANPEDAAPVWYVDVTTNEVVVLASDVPAAEAFVAASGADASTVRVERSDESGLSRIVNGEEDAYYIGNGRCSIGFSVRQGSTPGFVTAGHCGSVGNATTGFNRVSQGTFRGSWFPGRDMAWVAVNSNWTPTSLVRNSGSGVRVTGSTQATVGSSICRSGSTTGWRCGTIQQHNTSVTYPQGTITGVTRTSACAQPGDSGGSFISGTQAQGVTSGGSGNCSIGGTTFHQPVNPILSQYGLTLVRS

SEQ ID NO: 73 - DNA, 735 bp, Bos taurus
  source 1..735: mol_type = genomic DNA
tgtggtgttcctgctattcaacctgttctttctggtctttctagaattgttaacggtgaggaagctgttcctggttcttggccttggcaggtttctttgcaggataagactggttttcatttttgtggtggttctttgattaatgagaattgggttgttactgctgctcattgtggtgttactacttctgatgttgttgttgctggtgagtttgatcaaggttcttcttctgagaagattcagaagttgaagattgctaaggtttttaagaatagtaagtataattctttgactattaacaacgatattactttgttgaagttgtcaactgctgctagtttttctcaaacagtttctgctgtttgtttgccatctgcttctgatgacttcgctgccggtaccacttgtgtcactactggttggggtttgactagatacactaacgccaatactccagatagacttcaacaagcttctttgccacttctttctaatactaactgtaagaagtattggggaaccaagattaaagatgctatgatttgtgccggagcctctggagtttcttcttgtatgggtgattctggtggtcctttggtttgtaagaagaacggagcttggactttggtcggaattgtctcttggggttcttctacttgttctactagtactcctggtgtttacgctagagttactgctttggttaattgggttcagcaaactttggctgctaat

SEQ ID NO: 74 - DNA, 756 bp, Sus scrofa
  source 1..756: mol_type = genomic DNA
tgtggtgttcctgctattgatcctatgttgtctggtttgtctagaattgttaacggtgaggaggctgttcctggttcttggccttggcaggtttctttgcagacttcttctggttttcatttttgtggtggttctttgatttctgaggcttgggttgttactgctgctcattgtgaggttagaaagtctcatcttgttgttgctggtgtttctgatttgtcttctgatgaggaggctgtccaggttttgagaattgctgaagttgttgagcatcctttgtggaacttgcatgctttgagaaatgatattgctttgttgaagttggctactcctgctagattgagtggtgctgtttctccagtttgtttgccttctactaatacttcttttcctactggttctttgtgtgctactactggttggggtaagactagacataacgctttgaagactccagataagttgcaacaggctgctttgcctattgtttcttccgctgactgtaagtcttactggggttctaaggttactgacgttatgatttgtgctggtgcttctggtgtttctacttgtatgggtgattctggtggtccattggtttgtcagaaggatggtgcttggactttggctggtattgtttcttggggttctggtagatgtttgcctttttctccaggtgtttacgctagagttactaagtttgttccttggattttggaggttttgaaggctaatcatcatcatcatcaccactaa

SEQ ID NO: 75 - DNA, 735 bp, Sus scrofa
  source 1..735: mol_type = genomic DNA
tgtggtgttcctgctattgatcctatgttgtctggattgtccaggattgttaacggtgaagaagctgttccaggatcttggccttggcaagtttctttgcaaacttcttctggatttcatttttgtggaggatctttgatttctgaggcttgggttgttactgctgctcattgtgaagttagaaagtctcatttggttgttgctggtgtttctgatttgtcttctgatgaagaagctgttcaagttttgagaattgctgaagttgttgaacatccattgtggaacctccacgctttgagaaacgatattgctttgttgaagttggctactccagctagattgtctggtgctgtttctccagtttgtttgccatctactcaaacttcttttccaactggatctttgtgtgctactactggatggggtaagactagacataacgctttgaaaactcctgataagttgcaacaagctgctttgcctattgtttcttctgctgattgtaagtcttactggggatctaaggttactgatgttatgatttgtgctggagcttctggagtttctacttgtatgggagattctggaggaccattggtttgtcaaaaggatggtgcttggactttggctggtattgtttcttggggatctggtagatgtttgccattttctccaggagtttacgctagagttactaagtttgttccttggattttggaagttttgaaggctaac

SEQ ID NO: 76 - DNA, 735 bp, Gadus morhua
  source 1..735: mol_type = genomic DNA
tgtggtagacctgctatttctcctgttattactggttattctagaattgttaatggtgaggaggctgttcctcatagttggagttggcaggtttctttgcaggatcagactggttttcatttttgtggtggttctttgattaatgagaattgggttgttactgctgctcattgtaatgttaagaattaccatagagttgttttgggtgagcatgatagatcttctaacagtgagggtgttcaagttatgactgttggtcaagtttttaagcatccaagatacaatggttttaccattaacaacgatattttgttggtcaagttggcaactccagccactcttaacatgagagtttctccagtttgtttggctgaaaccgatgatgtcttcgaaggtggtatgaagtgcgttacctccggttggggattgactagatacaacgctgccgatactccagctttgttgcaacaagctgctttgcctttgttgacaaacgagcaatgtaagaagttttggggtaacaagatttctgatttgatgatttgtgctggtgctgctggtgcttcttcttgtatgggtgattctggtggtccattggtttgtcagaaggccggttcttggactttggttggtattgtttcttggggttctggtacttgtactcctactatgcctggtgtttatgctagagttactgagttgagagcttgggttgatcagactattgctgctaat

SEQ ID NO: 77 - skipped

SEQ ID NO: 78 - DNA, 735 bp, Danio rerio
  source 1..735: mol_type = genomic DNA
tgtggagttcctgctattccaccagttgttactggatacgctagaattgttaacggtgaagaagctagaccacattcttggccctggcaagtttccttgcaagattctactggatttcatttttgtggtgggtctttgattaacgaaaactgggttgttactgctgctcattgtaacgttagaacttcacatagagttattttgggtgaacatgatagatcctctaacgctgaagctattcaaactattgctgttggtaagtcaattaagcatcctaactacaactcttttactattaacaacgatattttgttgattaagttggctactccagctaagattaacactcatgtttcacctgtttgtttggctgaaactaacgataactttccaggaggtatgaagtgtgttacttccggatggggattgactagatacaacgctccagatactccagctttgttgcaacaagctgctttgccattgttgactaacgatgattgtaagagatactggggtactcaaattactgatttgatgatttgtgctggtgcttcaggagtttcttcctgtatgggagattccggaggaccattggtttgtgaaaacaacagagtttggactttggttggaattgtttcatggggctcttccacttgttctacttccactccagctgtttacgctagagttactaagttgagagcttgggttgatcaaactattgcttcaaac

SEQ ID NO: 79 - DNA, 657 bp, Beauveria bassiana
  source 1..657: mol_type = genomic DNA
attgcaggagggcaagatgcagaacttggctggtttccatacacggtctcgttaagaaatcgaggtagacataattgtggtgggattatcatagaccaatcatcaattctgactgcagcccactgtatagttggtctaagcgctgacagagtttctatcgtggctggtgctgtcaaattggacagtggaggtaccacttaccaagtatcagctattcacaagcaccctggatacaattcgttgaccataacaaatgatatagctgtactgaagctcgcaaaaccgttgagttacacgccctatataaggcctatagccttggccactcaggatccccctgctggttcaggtactctgggttctggctggggggtgtcgagatcgtccggtcctgtctctaatacattacagtggatctctctgcaatcattggatcacaagatctgccaggagaaaatgggcgttactgtttcccagatctgcactactagcgctaacaaagggggtatgtgtcgtggggactctggtggtccgctaactgatgagagaatcactcttgtaggtgtgtacagttggggacgtggatgcggggacggatacccagacgtgtacgcctctgtggcatatcaccgagagtggatcagatcctatatggga

SEQ ID NO: 80 - DNA, 714 bp, Daboia siamensis
  source 1..714: mol_type = genomic DNA
caaaagtcttcagagcttgttgtcggcggtgacgagtgcaacatcaacgagcacagatcactggtgttcctctacaacaactctttcggatgttctggtactcttatcaaccaacaatgggtgctttccgcagtccactgtgatatggaaaacgttcgtatctacttgggtgtacacaatctacgtctcaggaacaatgctgaaatcagattgccagaagagagattcttttgtctaagttctaagtcttatactaaatgggacaaggacataatgctgattaagctggacagaccagttaaaacctccacttacatcgcccctctttccctcccctcttctccaccaagagtcggttctgtctgtagaattatgggttggggtgctattacgtctcctcaggaaacatttccaggcgttacccactgcgcaaacatcaacattctcccttactctgtttgtagagccgcctacaagggtttacctgctcagtccagaaccctatgtggaggtattctggaaggtggtattggttcctgtatgggagacagcggaggtcctctaatctgtaatggtgagatgcacggaatcgttgcttggggagacgacacttgcgctcaaccacataagccagttcattatactaaggtctacgattacactgattggattcaatcgattattgctggtaacaccgcagctacctgccctcca

SEQ ID NO: 81 - DNA, 717 bp, Macrovipera lebetinus
  source 1..717: mol_type = genomic DNA
caaaagagttctgaattagttgtcggtggtgatgagtgtaatataaatgagcatcgtagtttggtgttcttatacaattcttcgtttggatgtggtggcactttgatcaatcagcaatgggtcttgtctgctgcacattgtgacatggagaacgtacaaatttacttgggcctgcataacctaagattaagaaaccaagatgagcaaattcgtgtagccgaagagaagttcttttgtctgtctaacaagtcatataccaaatgggataaagatattatgttgattagactgaactcctctgtaacctacaacacgcacattgctcctctttccttgccatcttctccaccccgagttggatcagtctgcagaattatggggtggggagctattaccagtccaaatgagacatttcctaacgtgccacattgtgccaatattaatatcttgagatatagcgtctgtagggcagcctacagaggactgcctgcccagtccagaaccctttgtgccggcattctgcagggaggtattggaagctgtatgggtgattctggaggtcctttaatttgcaacggtgagatccagggtattgtcagctggggtgatgacatatgtgcccaaccccataaaccagtacattatacgaaggttttcgattattcggattggatccagtccataatcgcagggaatacgaccgctacgtgcccattg

SEQ ID NO: 82 - DNA, 717 bp, Macrovipera lebetinus
  source 1..717: mol_type = genomic DNA
cagaagtcctcggagcttgttgtaggtggagacgaatgtaacatcaatgaacatagaagcttagttttcctataccaaagttcttttggatgtggaggcacacttattaatcagcaatgggttttatcagcagcacattgtgatatggagaatgtccaaatatatttaggtttgcataatctaagattaagaaatcaggacgagcaaatacgtgtggcagaggagaagtttttctgtctcagtcagaaaagctacacaaagtgggacaaagacatcatgctgattagactccagtcctctgtaacctacaacactcacatcgctccactgagcttgccatcttcccctccgagagtgggatccgtgtgtcgtataatgggatggggcgctatcacttcaccacaggagactttccctaacgtccctcattgtgctaacattaatattttgcgttactctgtatgtcgtgctgcttacaggggcttgcctgctcagtctaggactctttgtgctggtattttgcaaggaggtatcggttcttgtatgggagatagcggtggccctcttatttgtaatggtgaaattcaaggtattgtcagttggggcgatgacatatgtgctcaacctcataaacctgtccactacaccaaagttttcgactatagtgactggattcagagcattattgctggtcaaacaactgctacatgtcccctg

SEQ ID NO: 83 - skipped

SEQ ID NO: 84 - DNA, 720 bp, Macrovipera lebetinus
  source 1..720: mol_type = genomic DNA
gagaagtcttccgaactcgttatcggggggaggccttgcaacattaaccaacatagatccttagctttattatataattcaagtggtttcttatgcggtggtactcttataaatcaacagtgggttctgtctgcagcacattgtgatatggagaatatgcaaatttacttgggattgcataatttttctctcccaaatatggatcaaaagcgtagggtggctgaagagaagtttttttgtctgtctaataaatcctacaccaagtgggacaaggatattatgctaataaaattaaatagaagagtcaagacctcaacccatatcgcaccactctcgctgcctagtaatccacccagattgagatccgtttgcagaattatgggctggggttctatcacctcgcctcgtgaaaccctaccttatgttccgcactgtgccaatattatgatcttgagatattgggtttgtagagcaatatacgggtctttgccagcaaagtcgcgaacgttgtgtgctggagttccacgacgtaggattggttcatgccttggagattcgggtggtccattgatatgtaatggtcaaattcagggaattgcatcctgggggtctgatccatgtgtcaaccacggagctccaggtgtttacacaaaagtttttgattacaatgactggattcaatctattatcgctggtaacactgccgcaacatgccctccc

SEQ ID NO: 85 - DNA, 720 bp, Macrovipera lebetinus
  source 1..720: mol_type = genomic DNA
gaaaagagttccgagctggtaattggaggtcgtccatgtaatattaatcagcacagatccctggctttgctttaccaatcatctggatttttatgcggtggaactctaatcaaccaacagtgggttttgtctgctgcccactgtgatatggagaatatgcagatctatcttggtttgcatcaatttagtctccctaacatggaccaaaagagaagagtggcagaagaaaaatttttttgtttaagccaaaaaagttataccaaatgggacaaggacattatgctcattaaattaaataggagagtgaaaaccagtacacacattgcaccgctcagcctgccttccaaccccccgcgactgagaagtgtgtgcagaatcatgggatggggttcaattactagtcctcgtgagacattaccctacgtgccccattgtgcaaatatcatgattttgagatattgggtttgtagagctatttatggcagtctgccagccaagagtagaaccctgtgcgctggagttccaaggcgtcgtattggatcctgcttgggagattccggtggaccacttatttgtaacggtcaaattcaggggatcgccagttggggatcagatccatgtgtcaaccacggcgctccaggagtatacactaaagtcttcgactacaatgattggattcaatcgattattgctggtaatactgcagcaacctgtccacct

SEQ ID NO: 86 - DNA, 735 bp, Capra hircus
  source 1..735: mol_type = genomic DNA
tgtggcgtacctgcgattcagccggttctgtcaggactcgctcgcatagttaatggggaagacgcagtccccggttcatggccttggcaagtgagcttgcaagattctacctcgtttcacttctgcggcggttcgttgatatccgaagactgggtcgtcactgcggctcattgcggggtcacaacatcggatgtagtcgtggctggggagttcgatcagggttcccagactgaaaatacccaagtcttaaaaattggcaaagtgtttaagaacccgaagttttctattctgacagttagaaacgatatcaccctgctaaaacttgccacgcctgcacagtttagtgagacggtgagcgcggtgtgcttacctagtactgatgaagacttcccagcaggtatgctctgtgcgaccaccggatgggggaaaacgaagtacaatgcattaaagacgccagacaagctacaacaagctacgcttcccatagttagtaacactgactgtaagaaatattggggttctcgagttacagacgtaatgatctgcgcgggggcatcaggagtttcttcctgcatgggggacagcggtggacccttggtatgtcagaaagatggcgtatggaccctcgccggaatcgtatcatggggctcctcgacgtgtagcactagtactccggctgtatatgccagggtcacagcgcttatgccatgggtgcaggagacactagccgccaac

SEQ ID NO: 87 - DNA, 735 bp, Rattus norvegicus
  source 1..735: mol_type = genomic DNA
tgcggggtccctaccattcagcctgttctgacaggtctatcgcgtatcgtaaatggcgaagacgccattccgggcagttggccgtggcaagtgtccctccaagacaaaaccggcttccatttttgtggtgggtccctcatttcggaagattgggtagtgactgccgcacactgcggggtcaagactagcgacgtagttgtcgctggagagttcgatcagggatcagacgaggagaatatccaagtccttaagattgctcaggtgtttaaaaatcctaagttcaacatgtttactgttcggaacgacataactctattaaagcttgccacgccagcgcaattctcagaaacggtttctgctgtctgcctcccaaatgtagatgatgattttcccccgggaacggtgtgtgcgaccactggatgggggaaaaccaaatacaacgccttaaaaacacccgaaaagttacaacaggcggcgctgcccatcgtgtccgaggcagattgtaaaaaatcatggggctctaagatcacagatgttatgacctgtgcaggtgcatctggagtaagtagttgcatgggtgactcaggcgggccactagtgtgccagaaggacggggtatggacgttggctggaatagtgtcttggggttcgggtgtctgttcgacaagcacgcctgccgtttatagccgagttacagcgttgatgccatgggtacagcaaatactggaggctaac

SEQ ID NO: 88 - DNA, 735 bp, Oryctolagus cuniculus
  source 1..735: mol_type = genomic DNA
tgtggggttccagcgatacagccagtcctgacgggattgtctcggattgtaaacggtgaggatgccgttcccgggtcgtggccgtggcaagtgagccttcaagataagacgggattccacttctgtggaggatcgctaattagcgaaaattgggttgtaacagccgctcattgcggtgtgagtacttcgcacttggtggttgcaggggaatttgaccaaggttccagtcaagaaaaggtgcaggtcctcaaaatagctaaggtatttaagaaccctaagttcaatatttttactatcaggaacgacattaccctattaaaacttgctacccctgcctccttttcacaaaccgtatccgcggtttgccttccaaacgcggatgacgacttccccgctggcaccctctgcgctactacaggctggggtaaaatccgttacaatgccaacacaacacctgaaaagctgcaacaggcggcactaccgttactgtctaacgcggattgtcagaaatattggggcactaaaataacctcagtaatgatctgcgccggtgcaaatggggtgtcaagttgtataggtgattctggcggccccctcgtatgccagaaaaatggggcctggacgttagtcggaattgtttcttgggggagcaatacatgtagcacttccacgccggcagtctatacgcgagtcaccgagttgataccgtgggtccaggagatcctcgctgcaaat

SEQ ID NO: 89 - DNA, 735 bp, Balaenoptera acutorostrata
  source 1..735: mol_type = genomic DNA; sub_species = scammoni
tgtggggtgcccgcgatagacccggttctcagcgggttaagcagaatcgtgaatggagaagatgcggtccctggttcgtggccgtggcaagttagtctgcaggacagcacaggctttcacttttgcggcggctcgctaatctctgaggattgggtggttactgccgcccattgtggtgtcaagacgagcgacctagtcgtcgctggggaattcgatcagggttctgatgctgagaacatccaggtacttaagatagcgaaagtatttaagaacccaaagttcaacatgctgacagtcaggaatgatattacgctgctgaaattggctactccggcacggttctctgaaaccgtttctcctgtgtgcctaccaagtgcaaacgacgatttcccagccggtatgttgtgtgtaacaacgggctgggggaagactcgctataatgccccaaaaacacccgacaaacttcaacaagctactcttcccatattatcaaacgccaattgtcgtaaatactggggaaataagatcaccgatgtaatgatatgcgcaggcgcatcgggagtatcatcttgcatgggcgactcggggggacctctcgtgtgccagaaagacggtgcttggactctcgttgggattgtctcctggggttcaggtacctgttccacgagtacccccgcggtatatgcccgagttaccgcgttgattccgtgggtgcaaaaaattttagcagcaaac

SEQ ID NO: 90 - DNA, 735 bp, Gallus gallus
  source 1..735: mol_type = genomic DNA
tgtggtgttcctgcgatcacgcccgtcattcgcggctacaataggattgtgaatggagagccggctgtccctggtagctggccgtggcaagtaagtctgcaacgttatggtaatttccacttttgtggcgggtcactaatctcggagcagtgggtcgttacggcggcgcattgcggcgtgaggaccaccgacactgtagtgcttggcgaatatgatcaagagacggcctcctcggatgtgcagcgattggggatcgctaaggtctttcggaacccatcttacagctcattaacaatcagaaatgatattaccttaattaaacttgcgaccccagcccagttgaacgcacgcgtagctccagtatgcttgccccaagctactgatgacttcccaggcggattgacatgtgtaacgacaggatggggcctgcttaactctaacgacgaaaatacacccgctatactgcaacaggccgcactcccgctattaacaaacgcccagtgcaagcagtactggggatttcgtatatatgacgttatggtttgcgcaggagcggatggttcttcttcctgtatgggggactcgggtgggcctctggtctgccaaaaggacggagcatggactctcgtggggatagtttcctggggtagtagtacgtgttcaactagtacccccggtgtgtacgcccgagtaactaaactacgggaatggatagattctgtcctcgccgcaaac

SEQ ID NO: 91 - DNA, 735 bp, Crocodylus porosus
  source 1..735: mol_type = genomic DNA
tgtgggactcctgcgatacagccgattatctctggatacgcaagaattgttaatggagaagaggccataccgggttcttggccatggcaggtttccttacaagacaacactgggttccatttctgtggcgggtcgctcatcagtgaggattgggtcgtgacagcggcacactgtggtgtaaaaacgacggatcgggttgtacttggtgcttttgacctgggattccttggcgaagatgtgcaggttctaaaaatcgctcaagtattcaagaacaggaattttaacatgttgactctccgtgacgacattaccctattaaagctggcgacgccagcgaaaatgacggcccgggtctcccccgtctgcttacccgaggctaccgaatatttttcaggcggtattacctgtgtgaccacggggtgggggctcacaaacccgaacgcgcagaagacacccgacaagctgcaacaagtagcattgcctctcctaacaaacgtgcagtgcaaaagctattggtcttaccgtattaaagattgcatggtgtgtgctggtgccaaaggggcatcgagctgcatgggcgattcaggcggaccactggtctgtcaaaaggatggtatatggactcttgtcggtatagtcagttggggaagcaatacctgcagtccttcttcaccgggcgtttacacacgcgtatcgaagttgcgaccctggatcaatgaaataatcgcagccaat

SEQ ID NO: 92 - DNA, 729 bp, Bombus vosnesenskii
  source 1..729: mol_type = genomic DNA
tttcccgaaggtcagatcgtgggcggcaaggatgcgcctgttggaaagtttcctcaccaagtgtccttgcgtcagagcgggtcacatttttgtggggggagtattatcgactctaggcatattcttacggcagcgcactgcgttcaaggcctaaataatctcaaaagcatcaccgtgcatgccggtactaaccagcttaataccaacggtcagtcgtatggagtagaaaaagcagtcgcgcacaaggggttcaatagtaataccctcctgaacgacattgctataattcgcgtaaatcaaaatattgctttcaacaatctagtcaaatcgataaaactagccagcggctccaacacatatgagggttcaaactgcatcttgtcggggtggggaacaacgcggctgggcgggaagagtccgaataacctccaatacataaatttattagtagagagccagtcaaagtgtaaacaagcacattggcgagttcaatcgtctcacatctgcacatatactaaagtcggggagggcgcctgccacggagatagtggaggtccgctgatacatgatgatgttcaaattggagtggtatcttttggtcagccctgtgcggtaggtaagccagacgtgtacactagagtctcatctttcataccatggataaactcccagaaatcctacttgatgaacgaaacggaagagcaaccggaggacgcaatctatatcgct

SEQ ID NO: 93 - DNA, 735 bp, Apis cerana
  source 1..735: mol_type = genomic DNA
tacggatttcccaataaccaaatcgtagggggtaaggacgcaccggtagggaagttcccataccaggtttcgctccgaaaaaacaagaaccacttctgtggcggatctattatagacagccgcaccattttaacagcagctcattgcgtggagggtctttcgaacctgaatggcatcactgtacaagccgggacgaatcaactaaattccggtggcgtgtcttatgttccggagaaagttgtggcgcactcaggttttaattcactgagcttagttaacgatattgccctcatccgtgtcaaccaggctatatcgttcaataacctagtccaacccataaaattggcgagtggaacaaatacttacgaaggatcaagttgcgtcttgtccggatgggggacggttaaactcaatgggaacgtacctaacaatcttcaatatattaacttactaatcgagaaacaatcggaatgtaagaaggtgcactggagagtccaatcttctcatatttgtacctttaccaagtatggcgaaggggcgtgccatggtgattcaggcgggccattggtcgtcaatggcgtccaggtgggcatcgtttcgttcggtcaaccgtgcgcagtgggtaaacccgatgtatatacgcgggtatcttctttcagtagctggatacagcagcataagtcctttcttatggaggaaaaatccgaggctcctgccgacggagtttacataagt

SEQ ID NO: 94 - DNA, 687 bp, Solenopsis invicta
  source 1..687: mol_type = genomic DNA
tatggggtgcccgagagcaagatagtagggggcaaggacgcccccgtgggaaaatacccatatcaagtctctttaagactgagtgggagccatcgatgcggggcttcaatattggacaataacaacgtcttaactgcagcgcactgtgtagatggactctccaatctgaacagactaaaggttcatgttggtactaattacttgtctgagtcaggggatgtgtacgacgtggaagacgctgttgtgaataaaaactatgatgattttcttctccgcaacgatgtagcactagttcatctgacaaacccgattaaattcaacgacttggtgcagcctatcaaactgagcacaaacgatgaggatttagaaagtaatccatgcacgctaacaggttggggttcgacgcgactcggaggtaatacccctaatgcgttgcaggaaattgaacttatagtccacccccaaaagcagtgtgagcgggaccaatggcgtgttatcgactcgcacatatgcactcttaccaaaaggggagagggcgcctgccacggagactcgggaggcccgttagtcgccaatggcgctcagatcggcattgtatcttttggctccccatgtgcgctcggtgaacctgatgtatatacgcgcgttagttcattcgtctcctggattaacgcaaatcttaaaaag

SEQ ID NO: 95 - DNA, 738 bp, Danaus plexippus
  source 1..738: mol_type = genomic DNA
ttcattcttccacagttttacgatagcccatcaccccgcatagttggaggggagaacgcgcctgttggatctgcgccacaccaggcgtctctaagatcaatcttcggatcgcatttttgtgggggctcgattatttccaagcgttggattctaactgcagctcattgtacggtaggacaaagtacgtttactattaaagtagtggtggggactaattcactgacaagcggtggcgaaaagtactcagtagagaaaataatcgtccacgaggactacgacggtggagagatcataaacgatgtctcggtcgtcaaggtgagcacggacatcgaatttaccgatttagttcagcccattcaactgcctaaaaccgacacagccgaaggtgctgtgttgacccttacaggatggggccgaacgtcgtacccgggctcactcccggatcggctccaagttatcagtctcattgccctgagtgttaatcaatgccagcaaatatataaagggatcgtaagttctaatgtatacgatacacagatatgcagcctaactaagtccggggagggcgcatgccacggtgactccggtggtcccttggtcgaaggggacaaagtggtcggaattgtatcttggggcgtgccgtgtgctcgtggctatcctgatgtttataccagggtgtatagcttcaaggactggatattatccaagactaacatgacagatgaatccatcataaat

SEQ ID NO: 96 - DNA, 720 bp, Photinus pyralis
  source 1..720: mol_type = genomic DNA
gcggtatccccttttaacaacttattcggaactgttccaaaagacgagtttgattggaaaatagtaggtggttctgatgctcccgaaggagcttacccttaccaggtttctctgcggaggtttggcagtcactcttgcgggggttcgattgtggacgcaacaaccgttatgacagcagcccattgtgtcactggggcaaagccgtccgcactccgtgtggtcgtcggctctaataaactaaactcaggcggagtgtcatatgagatcgaatcaatacgggatcacaagggttttgatagctggagactaaccaatgatattgctatccttaagctcaagactccgataacgttcacggacaaggtaaaaacggtagatctggagacggaatatctcggcgcggaagtagattgcgtgcttagtgggtgggggagattgaactatcccgggtcattacctaataatctgcaacatatcggattgaagacaataacatccgaggactgtgccaaacttttaaatcactcgagcgttgatcgtacccatatatgcactttgaccaaatcgggcgagggtgcctgtcacggggacagcggaggcccactagtcgccaataaacagattggtattgttagcttcggacgaccctgtgctaagggatacccggacgcgttcaccagggtcagtgcgttcactgactggatcgaaaaaaaccgctat

SEQ ID NO: 97 - DNA, 711 bp, Aedes aegypti
  source 1..711: mol_type = genomic DNA
actttatccccagaatttcttgagtgggaaggaaggatagttggcggccaaaacgccggaagtggtcagttcccctggcaagtgagcctacgctcgagtggcaacgcgcacttctgtggtgggtcaatcatcaacaacagatgggtgttaagcgccgcacactgtacggttggacgaacactggcgaacacccgtgttgtcgtcgggacgcacttattgaacagcggaggagtagcgcataactcagctcggatagtaaatcacccatcgtataatgcgaattccctaacgaacgatatcagtctagtgcagaccgcctctattattgcatttaataatctggctcagccgatcggtctcggggcaacattcataaataccgcaagcggtgctttagcgtctggctgggggcaactgggtgccaacgtaggaatccctaacaatctccaatggctttcaacatccataataacgcttgcagactgccgaaatagacactcagtcgtcaatcgggggcgcgtgcatgacaacactatttgcaccctgaacccctctggtcagggtatgtgcatgggcgattctggggggcctttggttcatggtggaaatcagcaaggtgtcgtatcctgggggattccgtgtggcttgggctcgccagatgtgtttgctagggttagttcgcatcgtacttggattctcaataatgctgta

SEQ ID NO: 98 - DNA, 735 bp, Tenebrio molitor
  source 1..735: mol_type = genomic DNA
cttcccctgaatgagaagatcactcctttcaacgccacctcaggacggatcattaatggaagtccggcttctaaaggccaatttccttggcaggcagcagtgatattcacgacaagctcaggacagagcttttgcggggggtctatcatttcgaaccagtggatactgactgcggcgcactgcgctcgaggagccaccagtttcactatatacctcgggactaacacactaaccccagactctggttccacgactgtcacgacaacccgcgccgttatccacccaagctataatcccagcacgcttgctaatgacgtagcattgattcagttaacatcgccgataacctttaatacttacgttcaacctataacattagcaagttcgacgctaggctcgggagtgagcgtaactgtgtctggttggggaaagacatcagattccgcttcgggggttaccaacacgcttaactacgtaactctctcctccatcacgaacacacaatgtagtaattattatggtagtctgggtagtggggttgtttgtgcgaccggcgcgtccaaatccacgtgtaatggcgatagcggtggtcccttggtcactgggagtggatctaacgcagtgcatgtcggggtagtgtctttcgtctcgtcaaggggctgcgaatccggctacccatcaggttattcacgtacggcctactatcgagactggattagaacaaccacaggcgtc

SEQ ID NO: 99 - DNA, 714 bp, Tenebrio molitor
  source 1..714: mol_type = genomic DNA
tccccaatatcagagcgtatcgtgaatggcaccgacgctagagaaggcgagatcccttacatagtcagtctacgcctccaggatcgacacacttgcggggggagtatactggattcacggcacatcttaactgccgcacactgcgttcagtatgacgtcatctactacagcgttcagtacggtgtcacgcgtattagctctgcgttaaatgcgccgaatgtgatagaggtctccaaaattacagtccatgagggctatgttcctggtaatggttacctcaacgatgtggcagtggtagaattaaaaagcgacataccgttcggtgtacatgttcaaccggtgaaacttccttcgttggcagcggctacgccagaaaacgcccccgcaacgctatcaggctggggctatccatacaccggcggggccgtcatggaagatcttcaaacggtggatattgtagtcttttcggacgcggactgcgaacggattcatgccggaacaggacccacttatagtaactatcatgtgtgtgcagggattcccgaaggtggaaaaggacaatgttccggggactctggggggcccttggttgtagatggtgttcaggtcggcatcgtgtcttggtccgtaaagccttgtaccgttcagggattcccaggagtttttattaaggtatcgacacaactgggttggattcaaaggcaagtagctgct

SEQ ID NO: 100 - DNA, 723 bp, Tenebrio molitor
  source 1..723: mol_type = genomic DNA
actccagttaacgtcgactccaatatcgactggagggttgtgggcggatcaacggcgacaccgcaccagttcccttttatcgttagcctacggacgccttatgatagtcataactgtggaggctcaataattgcaaaaaattatgtgattactgctgcacattgcgtcagcgggtacgcaccaagctactatacagtcgtagcgggcacgaatcaactgaacgccacaaacccattacgactgaaagtcgcacagattatcgtacacccagagtacagcagttccttgatactaaatgatgtggcccttctgaggttggaaacccccgtcgaagaatcagaagaggttcagatcgtaggtctcgagaccgaatacgtagacactgtacgagattgtgtccttgttggttggggtagaacctcttatccgggatctatccccaacgatctacaatttctcaatgaacggacctatccgaatgacgagtgtgttaatcgctgggcttcggcccatgcggtatactcttcacaaatatgcaccctcacaaaggttggcgagggtgcctgccacggggattcggggggaccccttgtggtagtgaaggacgataaattttccttaatagctttggtctcttggggctcgccctgcgcgcgtgggatgcctgacgtgtacacgcgcgtggctagtttccacgagttcattactgataacattaagaac

SEQ ID NO: 101 - DNA, 756 bp, Diatraea saccharalis
  source 1..756: mol_type = genomic DNA
gagcagattcgtgaacgagaggaaaggtcattgtcacagcgcattgtgggagggtcctccgcggaggaaggccaattcccctatcaagcagggctcctaaccgatatgattggcctacaaggaaatggagtttgtggtggctcgctagtcagtgcgaatcgagtcgtgacagctgcccactgctggttcgatgggatgcaccaggcttggcgcgtcaccgtgattttggggtctactcggcttttcagtggtggacagcgactggcgacaagcgcagtcgttacccaccctggttggcaacctcccctcatccggcatgacgtagcgataatctacttaccaagcaatgctgttatgtcgaacacagtggctccgatcgcattgccgtcaggtgacctgcttaatcaaaattttgtgggagaactggccaccgcttcaggctttgggttaacttctgacaacgccaacataacgactaatcaatttcttagccatgtcaacctaaacgtaatctccaataacatttgccgtttcagcttcccattattaatacaggaggatcatatatgtacgtctggccttggtggagttgggacgtgtagaggagacagtggtggccccctcgtaatctttaggaacaatagtccgattctgatagggattacatctttcggttcccctttgggctgcgagtcgataatgccagccgcatatgccagagtaacggaatacgttgattttatcaaccagcacctc

SEQ ID NO: 102 - DNA, 702 bp, Apis mellifera
  source 1..702: mol_type = genomic DNA
gagaatctggaaaaaatcgtaggtggaaccaacgccagtcctggacaatttccataccaggtttctcttagaaagagtggtcgtcatttttgtggtggtacattgattactgagcgtcatatcgtcaccgcagcacattgcatccacggcatagtgtccgccccatataacgactttacggtggtgacaggtactatatctaacatcaacggtggtcagtcttactgcgtggcaaaggcaactgtcaatcctgacttcaaaccttcctctagtgagtcatatcgtaatgacattgccatagttacattggctgacacagttaagtctaacacttaccaaaagcccattagtccagcctcatctgatccccctgtcggcgccacgctgatcatgtccggttggggccgtacctcaacgaacggaaatcttcccgagatccttcaaacgaccaacgtctatctaatgtcaaatgaggagtgccaaaagcgtatcccaaattaccatatctacaatggtcaattatgtacatttaagcgtaaaggtgttggtatctgcatgggagattccggtggccctctggtatataacggtgagcttattggaattgcatcatgggtaattccttgcgcacagggataccccgacgcatatactagagtgactcagtacagaaactttatcaatcaaatagtgtctaacatc

SEQ ID NO: 103 - DNA, 714 bp, Apis mellifera
  source 1..714: mol_type = genomic DNA
ctgagttggaattacaatcccaaaattatcaatggtgaagacgctaaggagggagaaatcccatatcaagtgagtcttcaaaataagttttccagttttcatttttgcggaggctcaattcttaatgagaattacgtgataacggcagcacactgtgtccacggaaaattttccgaggacattaaagtagtggcaggcacgattaacttagccaatcccagatacgaaaatgacgtcaacgagatcattgtccacgaaaagtataacgtctccgattcttggaaaaatgacattgcccttctgaaagtaaagaccagttttactctttccaacacaatttctagtgtacctctgccatcaccaaacgacatcattaagcctaatgatcttgctaccgttagtggctggggaaggctaaggcaaggtggtccaactacaatttatttgcaaagagtcaacattttgatcgcaaaccaagaatactgtgagctaacttataagaagatcaattacacggtgtacgaatctcaaatctgtgcctattatcctacttccgagaagggttcatgtaacggagattcaggcggcccattgacggtcaacggtaagcttgttggactggttagttgggctatgggctgcgcccttatagactatccaacagtttataccagagtggaatcttaccttgactggataaaggaaaatgctatt

SEQ ID NO: 104 - DNA, 735 bp, Apis mellifera
  source 1..735: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgttgttggagacttgcaggtcggtattgtttcttttggtcagccatgtgctgttggtaagcctgatgtttacactagagtttcttcttttacttcttggattaatgagcataaatcctttttgatggaagaaagaggtgaggctccagctgatggtttttacatttct

SEQ ID NO: 105 - DNA, 735 bp, Apis mellifera; identical to SEQ ID NO: 104
  source 1..735: mol_type = genomic DNA

SEQ ID NO: 106 - DNA, 738 bp, Apis mellifera
  source 1..738: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggatcttgtcacggagattctggtggtcctcttgtttgtggagacttgcaggtcggtattgtttcttttggtcaggacatttgtgctgttggtaagcctgttgtttacactagagtttcttcttttacttcttggattaatgagcataaatcctttttgatggaagaaagaggtgaggctccagctgatggtttttacatttct

SEQ ID NO: 107 - DNA, 735 bp, Apis mellifera
  source 1..735: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgatgctattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgttgttggagacttgcaggtcggtattgtttcttttggtcagccatgtgctgttggtaagcctgatgtttacactagagtttcttcttttacttcttggattaatgagcataaatcctttttgatggaagaaagaggtgaggctccagctgatggtttttacatttct

SEQ ID NO: 108 - DNA, 735 bp, Apis mellifera
  source 1..735: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgatgcttgtttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctctttgtgttggagacttgcaggtcggtattgtttcttttggtcagccatgtgctgttggtaagcctgatgtttacactagagtttcttcttttacttcttggattaatgagcataaatcctttttgatggaagaaagaggtgaggctccagctgatggtttttacatttct

SEQ ID NO: 109 - DNA, 735 bp, Apis mellifera
  source 1..735: mol_type = genomic DNA
tacggtgttccagaatctaagattgttggtggtaagaacgccttgtctggtcaatttccataccaagtttctttgagaaagaacggatctcatttttgcggtggttctattattgattctagaactattttgactgctgctcattgtgttgaaggtttgtctaacttgaacggtattactgttgttgctggtaccaaccaattgaactctggtggtgttagttacgttccagagaaggttgttgctcatccatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgtttgtggagacttgcaggtcggtattgtttcttttggtcagccatgtgctgttggtaagcctgatgtttacactagagtttcttcttttacttcttggattaatgagcataaatcctttttgatggaagaaagaggtgaggctccagctgatggtttttacatttct

SEQ ID NO: 110 - DNA, 735 bp, Apis mellifera; identical to SEQ ID NO: 109
  source 1..735: mol_type = genomic DNA

SEQ ID NO: 111 - DNA, 735 bp, Apis mellifera
  source 1..735: mol_type = genomic DNA
tacggtgttccagaatctaagattgttggtggtaagaacgccttgtctggtcaatttccataccaagtttctttgagaaagcaaggatctcatttttgcggtggttctattattgattctagaactattttgactgctgctcattgtgttgaaggtttgtctaacttgaacggtattactgttgttgctggtaccaaccaattgaactctggtggtgttagttacgttccagagaaggttgttgctcatccatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgtttgtggagacttgcaggtcggtattgtttcttttggtcagccatgtgctgttggtaagcctgatgtttacactagagtttcttcttttacttcttggattaatgagcataaatcctttttgatggaagaaagaggtgaggctccagctgatggtttttacatttct

SEQ ID NO: 112 - DNA, 735 bp, Apis mellifera
  source 1..735: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggaccatgtcacggagattctggtggtcctcttgttgttggagacttgcaggtcggtattgtttcttttggtcagccatgtgctgttggtaagcctgatgtttacactagagtttcttcttttacttcttggattaatgagcataaatcctttttgatggaagaaagaggtgaggctccagctgatggtttttacatttct

SEQ ID NO: 113 - DNA, 735 bp, Apis mellifera
  source 1..735: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagtttgtcacggagattctggtggtcctcttgttgttggagacttgcaggtcggtattgtttcttttggtcagccatgtgctgttggtaagcctgatgtttacactagagtttcttcttttacttcttggattaatgagcataaatcctttttgatggaagaaagaggtgaggctccagctgatggtttttacatttct

SEQ ID NO: 114 - DNA, 738 bp, Apis mellifera
  source 1..738: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgttgttggaggtccattgcaggtcggtattgtttcttttggtcagccatgtgctgttggtaagcctgatgtttacactagagtttcttcttttacttcttggattaatgagcataaatcctttttgatggaagaaagaggtgaggctccagctgatggtttttacatttct

SEQ ID NO: 115 - DNA, 735 bp, Apis mellifera
  source 1..735: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgttgttggagacttgcaggtcggtattgtttcttttggtcagccatgtgctgttggtaagcctggtgtttacactagagtttcttcttttacttcttggattaatgagcataaatcctttttgatggaagaaagaggtgaggctccagctgatggtttttacatttct

SEQ ID NO: 116 - DNA, 735 bp, Apis mellifera
  source 1..735: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgttgttggagacttgcaggtcggtattgtttcttttggtcagccatgtgctgttggtaagcctcatgtttacactagagtttcttcttttacttcttggattaatgagcataaatcctttttgatggaagaaagaggtgaggctccagctgatggtttttacatttct

SEQ ID NO: 117 - DNA, 735 bp, Apis mellifera
  source 1..735: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattattactgaacaaaagtgtaagcaagctcattggagagttcagtcctctcatatttgtactttcaccaagtctggtgaaggagcttgtcacggagattctggtggtcctcttgttgttggagacttgcaggtcggtattgtttcttttggtcagccatgtgctgttggtaagcctgatgtttacactagagtttcttcttttacttcttggattaatgagcataaatcctttttgatggaagaaagaggtgaggctccagctgatggtttttacatttct

SEQ ID NO: 118 - DNA, 735 bp, Apis mellifera
  source 1..735: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttgaccaagtctggtgaaggagcttgtcacggagattctggtggtcctcttgttgttggagacttgcaggtcggtattgtttcttttggtcagccatgtgctgttggtaagcctgatgtttacactagagtttcttcttttacttcttggattaatgagcataaatcctttttgatggaagaaagaggtgaggctccagctgatggtttttacatttct

SEQ ID NO: 119 - DNA, 735 bp, Apis mellifera
  source 1..735: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgttgttggagacttgcaggtcggtattgtttcttggggtcagccatgtgctgttggtaagcctgatgtttacactagagtttcttcttttacttcttggattaatgagcataaatcctttttgatggaagaaagaggtgaggctccagctgatggtttttacatttct

SEQ ID NO: 120 - DNA, 690 bp, Apis mellifera
  source 1..690: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgttgttggagacttgcaggtcggtattgtttcttttggtcagccatgtgctgttggtaagcctgatgtttacactagagtttcttcttttacttcttggattaatgagcataaatccaacatt

SEQ ID NO: 121 - DNA, 735 bp, Apis mellifera
  source 1..735: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgttgttggagacttgcaggtcggtattgtttcttttggtcagccatgtgctgttggtaagcctgatgtttacactagagtttcttcttttacttcttggattaatgagcataaatcctttttgatggaagaaagaggtgaggctccagctgatggtgctagaatttct

SEQ ID NO: 122 - DNA, 735 bp, Apis mellifera
  source 1..735: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctattgaaggagcttgtcacggagattctggtggtcctcttgttgttggagacttgcaggtcggtattgtttcttttggtcagccatgtgctgttggtaagcctgatgtttacactagagtttcttcttttacttcttggattaatgagcataaatcctttttgatggaagaaagaggtgaggctccagctgatggtttttacatttct

SEQ ID NO: 123 - DNA, 738 bp, Apis mellifera
  source 1..738: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaatcctcttgtcacggagattctggtggtcctcttgtttgtggagacttgcaggtcggtattgtttcttttggtcagtctacttgtgctgttggtaagcctgttgtttacactagagtttcttcttttacttcttggattaatgagcataaatcctttttgatggaagaaagaggtgaggctccagctgatggtttttacatttct

SEQ ID NO: 124 - DNA, 735 bp, Apis mellifera
  source 1..735: mol_type = genomic DNA
tacggtgttccagaatcttacattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgttgttggagacttgcaggtcggtattgtttcttttggtcagccatgtgctgttggtaagcctgatgtttacactagagtttcttcttttacttcttggattaatgagcataaatcctttttgatggaagaaagaggtgaggctccagctgatggtttttacatttct

SEQ ID NO: 125 - DNA, 735 bp, Apis mellifera
  source 1..735: mol_type = genomic DNA
tacggtgttccagaatcttacattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgttgttggagacttgcaggtcggtattgtttcttttggtcagccatgtgctgttggtaagcctgatgtttacactagagtttcttcttttacttcttggattaatgagcataaatcctttttgatggaagaaagaggtgaggctccagctgatggtgctagaatttct

SEQ ID NO: 126 - DNA, 690 bp, Apis mellifera
  source 1..690: mol_type = genomic DNA
tacggtgttccagaatcttacattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgttgttggagacttgcaggtcggtattgtttcttttggtcagccatgtgctgttggtaagcctgatgtttacactagagtttcttcttttacttcttggattaatgagcataaatccgctaac

SEQ ID NO: 127 - DNA, 735 bp, Apis mellifera
  source 1..735: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgtttgtggagacttgcaggtcggtattgtttcttttggtcagccatgtgctgttggtaagcctgatgtttacactagagtttcttcttttacttcttggattaatgagcataaatcctttttgatggaagaaagaggtgaggctccagctgatggtttttacatttct

SEQ ID NO: 128 - DNA, 684 bp, Apis mellifera
  source 1..684: mol_type = genomic DNA
tacggtgttccagaatcttacattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgtttgtggagacttgcaggtcggtattgtttcttttggtcagccatgtgctgttggtaagcctgatgtttacactagagtttcttcttttacttcttggattaatgagcataaatcc

SEQ ID NO: 129 - DNA, 735 bp, Apis mellifera
  source 1..735: mol_type = genomic DNA
tacggtgttccagaatctaagattgttggtggtaagaacgccttgtctggtcaatttccataccaagtttctttgagaaagcaaaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgttgttggagacttgcaggtcggtattgtttcttttggtcagccatgtgctgttggtaagcctgatgtttacactagagtttcttcttttacttcttggattaatgagcataaatcctttttgatggaagaaagaggtgaggctccagctgatggtttttacatttct

SEQ ID NO: 130 - DNA, 735 bp, Apis mellifera
  source 1..735: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgttgttggagacttgcaggtcggtattgtttcttttggtcaggcttgtgctgttggtaagcctgatgtttacactagagtttcttcttttacttcttggattaatgagcataaatcctttttgatggaagaaagaggtgaggctccagctgatggtttttacatttct

SEQ ID NO: 131 - DNA, 735 bp, Apis mellifera
  source 1..735: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgttgttggagacttgcaggtcggtattgtttcttttggtcagtgtccagctgttggtaagcctgatgtttacactagagtttcttcttttacttcttggattaatgagcataaatcctttttgatggaagaaagaggtgaggctccagctgatggtttttacatttct

SEQ ID NO: 132 - DNA, 735 bp, Apis mellifera; identical to SEQ ID NO: 127
  source 1..735: mol_type = genomic DNA

SEQ ID NO: 133 - DNA, 684 bp, Apis mellifera
  source 1..684: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgtttgtggagacttgcaggtcggtattgtttcttttggtcagccatgtgctgttggtaagcctgatgtttacactagagtttcttcttttacttcttggattaatgagcataaatcc

SEQ ID NO: 134 - DNA, 684 bp, Apis mellifera; identical to SEQ ID NO: 133
  source 1..684: mol_type = genomic DNA

SEQ ID NO: 135 - DNA, 735 bp, Apis mellifera
  source 1..735: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgttgttggagacttgcaggtcggtatttggtcttttggtcagccatgtgctgttggtaagcctgatgtttacactagagtttcttcttttacttcttggattaatgagcataaatcctttttgatggaagaaagaggtgaggctccagctgatggtttttacatttct

SEQ ID NO: 136 - DNA, 684 bp, Apis mellifera
  source 1..684: mol_type = genomic DNA
tacggtgttccagaatctaagattgttggtggtaagaacgccttgtctggtcaatttccataccaagtttctttgagaaagaacaagtctcatttttgcggtggttctattattgattctagaactattttgactgctgctcattgtgttgaaggtttgtctaacttgaacggtattactgttcaggctggtaccaaccaattgaactctggtggtgttagttacgttccagagaaggttgttgctcatagatcttttaacgctttgagtttggttaatgatattgctttgattagagttaaccaagatatttcttttactaacttgattcaaccaattaagttggcttcaggttctaagacttacgagggttctgattgtattttgtctggttggggtactactaagttgaacggtaacgttccaaacaacttgcaatggattaagcttaagattgaaactgaacaaaagtgtaagcaagctcattggagagttcagtcctctcatatttgtactttcaccaagtctggtgaaggagcttgtcacggagattctggtggtcctcttgttgttggagacttgcaggtcggtattgtttcttttggtcagccatgtgctgttggtaagcctgatgtttacactagagtttcttcttttacttcttggattaatgagcataaatcc

SEQ ID NO: 137 - DNA, 1185 bp, Gammaproteobacteria bacterium
  source 1..1185: mol_type = genomic DNA
tccgtttcacctagaattgttggtggaactttgtcccgagaaaacgcttggccatttatgactgctttgatgcaaaaggctgctgaattgaacattgctcaagaatcttacactgctttttttttggctggagttccacaaagatccttttcaggagctttggttaactgtgaacaaggatttgatgtttgttctaacgctaagggtaagatttgtttgattgaaagaggtattacttttttttccgaaaagattgctaactgtagaattggaggtggtattgctgctgttatttacaacaacgaagtttcctccttgtcatggagagctgataacgctaagattccagctgtttctatttcacaaaacgatggacaaattttgttgaaccatttgggagaacaaattcaatacgttcctttggatactattccaactacttccttttgtggtggatcttacattggtggtaagtgggttgttactgctgctcattgtgtttcacaagtttccgctgatttgattgctttgaacattggaggtcaaaacttggaaactgatcaagataacatttactacgctaagagaattgctttgcatcaaagatggaacccagaaactattgaaaacgatgttgctttgattgaattgactactgaacctcaaggtattactcctattaagattgctgatgaatcccaattgactcaagctattgaagattttactgaagttactgctattggtagaggatctaagttggctacttcttctaagttggttactgaattggaaagaccagatccattgttgtacgaagttcaattgtccgttgtttctaacagaaactgtgaaaagatttttgatgaatacgaaatttctaacggagctgatccaactattgttgatactactactaacattgcttcaggtatgttgtgtgctggtaacttggctggaggagaaggtacttgttttggtgattccggaggacctttggttttgaaccaaaacggtgaaaactacttgattggtttgacttcatggggagttggatgtgctcaaccagatttgtacggagtttactcccgcgccccacattacaaggattctattgaagctgttttgaacgatcaatacttgactagaccaactgattttccatccttgagacctagaaagtcc

SEQ ID NO: 138 - DNA, 1941 bp, Pseudoalteromonas sp.
  source 1..1941: mol_type = genomic DNA
aagcaagttactatttcacctgttaagccaaagattgttggtggtgaagaagctactgaaggtgtttacccctggatggctgctattgttgctactgctccatcagttgctacttccttgaaggttggagaaaactccattgaaacttcaccattttctttttcaccagctggagctgtttccggagaattggcttcctgtgaattgggagatgctgaatgtactgatgttgaagataaggtttgtttgattgaaagaggtactgttacttttgctgaaaaggctaacaactgtgaagctggtggtggagttggtgttgttatttacaacaacgttgaaggacatttttcaggaactttgggagaaggttttgaaggaactattccagttgttggtattactcaagctgatggtcaagcgttgttggatgaagttgctactgctgaaaaggctggttccactgctgctattgaagtttccgctgaagaaactttgactcaacaatcttcatgtggagcttcctttttgggtgatagatgggttttgactgctgctcattgtgttgattcacctaactctattaacatgaaggttaacgttggagaatttgatttggctgatggtgctgaagatgctattgaaattaagaacttgtacttgcatccaggatacgatactaacactttgcatgatgatattgctttgattgaattggctgaatccgttgataagccaactattaagattgctgatttggaattgacttctcaattggctactgataactccgttgctacttctattggttggggaggtagaactggatacttgccaggagaaggaccaacttccgattttccagaaattttgcatgaagttgatttgcaattgtactcctccttgcaatgtagaactattattgctgaatctcgaactagagatactgattttgaatttactgaagctgatgttgatgttactgaacaaatgatttgtgctggtactccagctggaggtttgtccacttgtcaaggagattcaggtggaccattgattgttaacactaacgatggatggcaacaagttggagttgtttcctggggtttgggatgtgctgctccaggatacccaggagtttacactagagctggtgcttaccaagattggatggatggtattactgatggtattgctattactcaattgcatgattttactactttgccagaaactgattccagaacttttaagttgatggttgaacaaaactccggagaagctgcttcagttacttacgaaattgaaggatcatccacttttactgttgaagaaggagcttgtaacactttggctgctggtgaaacttgtgaattgtccgctacttacactccagttgaagctggagcgcatgaggcttttgttgttattaagtccgataacgctgctttgaagtcctcaaagactaagttgatgggtaacgctttggcttcagcttcagatttgaaggatgcttccggttccacttccgaagaagtttccttgtttactggtggagatgttaagtgggaagaaaacgctgaaggaggtttgcaatccggtgatattgatgatggtcaagaatccattttgatggctgaagttaacggagaaggtaagttgacttttgaatggggtgtttcatccgaagaaaacgttgaagatccagctgaaccgtgggatgctttgtacttgactgttaacggagaattgcattcttacatttccggagatgttgcttttgaatccgttgaattggaattgccagaaggtaagaacttgattacttggtcatacattaaggatggagctgttactgaattggaagatactggatacgttagaaacattaactttgctaagaaggaacctattgttattcctcaaatgcctattactgaacaa

SEQ ID NO: 139 - DNA, 726 bp, Scytalidium lignicola
  source 1..726: mol_type = genomic DNA
gaattcgctccaggtggaaacggttttgctaggagacaagctcgtagacaagctagagctgctggtttgaaggcttctccatttagacaagttaacgctaaggaagctactgttgaatctaactggggaggtgctattttgattggttcagattttgatactgtttcagctactgctaacgttccatccgcttcaggaggttcctcagctgctggaactgcttgggttggtattgatggagatacttgtcaaactgctattttgcaaactggttttgattggtacggagatggaacttacgatgcttggtacgaatggtatccagaagtttccgatgatttttcaggtattactatttccgaaggagattctattcaaatgtcagttactgctacttcagatacttctggtagcgctactttggaacaattgactactggtcaaaaggtttctaagtccttttcccaagaatcctccggatctttgtgtagaactaacgctgaatttattattgaagattttgaagaatgtaactctcaaggatctgattgtgaatttgttccatttgcttctttttcaccagctgttgaatttactgattgttcagttacttccgatggtgaatctgtttccttggatgatgctcaaattactcaagttattattaacaaccaagatgttactgattgttccgtttccggaactactgtttcatgttcttacgtt

SEQ ID NO: 140 - DNA, 1065 bp, Metarhizium anisopliae
  source 1..1065: mol_type = genomic DNA
gctccaactcaagctgcttctcttcatccacaaattttggaagctatgaagagagatttgggtttgaatgctgaacaagctactgttagagttgctagagaaattcatgctactgatgttattgaacaattgagatcttccgttgcctttgccggtgcttggattgatgcagatgttttgtacattggtattactgatcaagctttggctgatgaagttactgctgctggtgctactccaattgttatgaccaactctttgtctaagttggaaaaggctaaggaagatttggataagattttcattggtagagctaacactttggaaactagttctgacacttcttctggtattgcatcttattttgttgatgttgctgctaacaagttggtcattgaagctttggctgactctcatggtcatgctgaacaattggccgctcaagttggtttgacttctgaatttgaagttagaactgttgagactatgccaactactatggcaactgttcagggtggtgatgtttattatattaatagatctagtagatgttctattggttttgctgttactactggttttgttagtgctggacattgtggtggttctggagcttctgctactacttcttctggagaggctttgggtactttttctggttcagtttttcctggttctgctgatatggcttacgttagaactgtctctggtactgttttgagaggttatattaacggttacggtcaaggtagttttccagtttctggttcttctgaggctgctgttggtgcttctatttgtagatctggttctactactcaagttcattgcggtactatcggtgctaagggtgctactgttaattaccctcaaggtgctgtttctggtttgactagaacttctgtttgtgctgagcctggtgattctggtggatctttttactccggttctcaagctcaaggtgttacttctggaggttccggtgattgttctagaggtggtactacttactttcagccagttaacagaattttgcaaacttacggtttgactcttgttactgct

SEQ ID NO: 141 - DNA, 1011 bp, Thermobifida fusca
  source 1..1011: mol_type = genomic DNA
caagaattggctttgaagagagatttgggtttgtctgatgctgaagttgcagaattgagagctgctgaagctgaagctgttgaattggaagaagaattgagagattctttgggttctgattttggtggtgtttacttggatgctgatactactgaaattactgttgctgttactgatccagctgctgttagtagagttgatgctgatgatgttactgttgatgttgttgattttggtgagactgctttgaacgattttgttgcttctttgaatgctattgctgatactgccgatccaaaagttactggttggtatactgatttggaatctgatgctgttgttattactactttgagaggtggtactcctgctgctgaggaacttgctgagagagccggtttggatgaaagagctgtcagaattgttgaggaagatgaagaaccacaaagtttggctgctattattggtggtaacccatattactttggtaattacagatgtagtattggttttagtgttagacaaggatctcaaaccggttttgctacagcaggtcattgcggttctactggtactagagtttctagtccttctggtactgttgctggttcttactttccaggtagagatatgggttgggttagaattacttccgctgatactgttactccattggttaatagatacaatggtggaactgttactgttactggttctcaagaagctgctactggttcttctgtctgtagatctggtgctactactggttggagatgtggaactattcaatctaagaaccaaaccgtcagatacgccgaaggaactgttaccggtttgacaagaactaccgcttgcgctgaaggtggagatagtggtggtccatggttgaccggttctcaagctcaaggtgttacttccggaggtaccggtgattgtagatctggaggtattactttttttcagccaattaacccattgttgtcatactttggtttgcaattggttactggt

SEQ ID NO: 142-144 - skipped (3 entries)

SEQ ID NO: 145 - protein, 89 aa, Bos taurus
MRFPSIFTAVLFAASSALAAPVNTTTEDETAQIPAEAVIGYSDLEGDFDVAVLPFSNSTNNGLLFINTTIASIAAKEEGVSLEKREAEA

SEQ ID NO: 146 - protein, 89 aa, Sus scrofa; identical to SEQ ID NO: 145

SEQ ID NO: 147 - protein, 89 aa, Sus scrofa; identical to SEQ ID NO: 145

SEQ ID NO: 148 - protein, 89 aa, Gadus morhua; identical to SEQ ID NO: 145

SEQ ID NO: 149 - protein, 89 aa, Gadus morhua; identical to SEQ ID NO: 145

SEQ ID NO: 150 - protein, 93 aa, Danio rerio
MRFPSIFTAVLFAASSALAAPVNTTTEDETAQIPAEAVIGYSDLEGDFDVAVLPFSNSTNNGLLFINTTIASIAAKEEGVSLEKREAEAYVEF

SEQ ID NO: 151 - protein, 89 aa, Beauveria bassiana; identical to SEQ ID NO: 145

SEQ ID NO: 152 - skipped

SEQ ID NO: 153 - protein, 89 aa, Macrovipera lebetinus; identical to SEQ ID NO: 145

SEQ ID NO: 154 - protein, 89 aa, Macrovipera lebetinus; identical to SEQ ID NO: 145

SEQ ID NO: 155 - skipped

SEQ ID NO: 156 - protein, 89 aa, Macrovipera lebetinus; identical to SEQ ID NO: 145

SEQ ID NO: 157 - protein, 89 aa, Macrovipera lebetinus; identical to SEQ ID NO: 145

SEQ ID NO: 158 - protein, 92 aa, Capra hircus
MRQVWFSWIVGLFLCFFNVSSAAPVNTTTEDETAQIPAEAVIGYSDLEGDFDVAVLPFSNSTNNGLLFINTTIASIAAKEEGVSLEKREAEA

SEQ ID NO: 159 - protein, 92 aa, Rattus norvegicus; identical to SEQ ID NO: 158

SEQ ID NO: 160 - protein, 92 aa, Oryctolagus cuniculus; identical to SEQ ID NO: 158

SEQ ID NO: 161 - protein, 92 aa, Balaenoptera acutorostrata; identical to SEQ ID NO: 158
  source 1..92: note = subspecies scammoni

SEQ ID NO: 162 - protein, 92 aa, Gallus gallus; identical to SEQ ID NO: 158

SEQ ID NO: 163 - protein, 92 aa, Crocodylus porosus; identical to SEQ ID NO: 158

SEQ ID NO: 164 - protein, 92 aa, Bombus vosnesenskii; identical to SEQ ID NO: 158

SEQ ID NO: 165 - protein, 92 aa, Apis cerana; identical to SEQ ID NO: 158

SEQ ID NO: 166 - protein, 92 aa, Solenopsis invicta; identical to SEQ ID NO: 158

SEQ ID NO: 167 - protein, 92 aa, Danaus plexippus; identical to SEQ ID NO: 158

SEQ ID NO: 168 - protein, 92 aa, Photinus pyralis; identical to SEQ ID NO: 158

SEQ ID NO: 169 - protein, 92 aa, Aedes aegypti; identical to SEQ ID NO: 158

SEQ ID NO: 170 - protein, 92 aa, Tenebrio molitor; identical to SEQ ID NO: 158

SEQ ID NO: 171 - protein, 92 aa, Tenebrio molitor; identical to SEQ ID NO: 158

SEQ ID NO: 172 - protein, 92 aa, Tenebrio molitor; identical to SEQ ID NO: 158

SEQ ID NO: 173 - protein, 92 aa, Diatraea saccharalis; identical to SEQ ID NO: 158

SEQ ID NO: 174 - protein, 89 aa, Apis mellifera; identical to SEQ ID NO: 145

SEQ ID NO: 175 - protein, 89 aa, Apis mellifera; identical to SEQ ID NO: 145

SEQ ID NO: 176 - protein, 89 aa, Apis mellifera; identical to SEQ ID NO: 145

SEQ ID NO: 177 - skipped

SEQ ID NO: 178 - protein, 89 aa, Apis mellifera; identical to SEQ ID NO: 145

SEQ ID NO: 179 - protein, 89 aa, Apis mellifera; identical to SEQ ID NO: 145

SEQ ID NO: 180 - protein, 89 aa, Apis mellifera; identical to SEQ ID NO: 145

SEQ ID NO: 181 - protein, 89 aa, Apis mellifera; identical to SEQ ID NO: 145

SEQ ID NO: 182-183 - skipped (2 entries)

SEQ ID NO: 184 - protein, 89 aa, Apis mellifera; identical to SEQ ID NO: 145

SEQ ID NO: 185 - protein, 89 aa, Apis mellifera; identical to SEQ ID NO: 145

SEQ ID NO: 186 - protein, 89 aa, Apis mellifera; identical to SEQ ID NO: 145

SEQ ID NO: 187 - protein, 89 aa, Apis mellifera; identical to SEQ ID NO: 145

SEQ ID NO: 188 - protein, 89 aa, Apis mellifera; identical to SEQ ID NO: 145

SEQ ID NO: 189 - protein, 89 aa, Apis mellifera; identical to SEQ ID NO: 145

SEQ ID NO: 190 - protein, 89 aa, Apis mellifera; identical to SEQ ID NO: 145

SEQ ID NO: 191 - protein, 89 aa, Apis mellifera; identical to SEQ ID NO: 145

SEQ ID NO: 192 - protein, 89 aa, Apis mellifera; identical to SEQ ID NO: 145

SEQ ID NO: 193 - protein, 89 aa, Apis mellifera; identical to SEQ ID NO: 145

SEQ ID NO: 194 - protein, 89 aa, Apis mellifera; identical to SEQ ID NO: 145

SEQ ID NO: 195 - protein, 89 aa, Apis mellifera; identical to SEQ ID NO: 145

SEQ ID NO: 196 - protein, 89 aa, Apis mellifera; identical to SEQ ID NO: 145

SEQ ID NO: 197 - protein, 89 aa, Apis mellifera; identical to SEQ ID NO: 145

SEQ ID NO: 198 - protein, 89 aa, Apis mellifera; identical to SEQ ID NO: 145

SEQ ID NO: 199 - protein, 89 aa, Apis mellifera; identical to SEQ ID NO: 145

SEQ ID NO: 200 - protein, 89 aa, Apis mellifera; identical to SEQ ID NO: 145

SEQ ID NO: 201 - skipped

SEQ ID NO: 202 - protein, 89 aa, Apis mellifera; identical to SEQ ID NO: 145

SEQ ID NO: 203 - protein, 89 aa, Apis mellifera; identical to SEQ ID NO: 145

SEQ ID NO: 204 - protein, 89 aa, Apis mellifera; identical to SEQ ID NO: 145

SEQ ID NO: 205 - protein, 89 aa, Apis mellifera; identical to SEQ ID NO: 145

SEQ ID NO: 206 - protein, 89 aa, Apis mellifera; identical to SEQ ID NO: 145

SEQ ID NO: 207 - protein, 89 aa, Apis mellifera; identical to SEQ ID NO: 145

SEQ ID NO: 208 - protein, 89 aa, Apis mellifera; identical to SEQ ID NO: 145

SEQ ID NO: 209 - protein, 89 aa, Gammaproteobacteria bacterium; identical to SEQ ID NO: 145

SEQ ID NO: 210 - protein, 89 aa, Pseudoalteromonas sp.; identical to SEQ ID NO: 145

SEQ ID NO: 211 - protein, 89 aa, Scytalidium lignicola; identical to SEQ ID NO: 145

SEQ ID NO: 212 - protein, 89 aa, Metarhizium anisopliae; identical to SEQ ID NO: 145

SEQ ID NO: 213 - protein, 89 aa, Thermobifida fusca; identical to SEQ ID NO: 145

SEQ ID NO: 214 - protein, 89 aa, Streptomyces griseus; identical to SEQ ID NO: 145

SEQ ID NO: 215 - protein, 89 aa, Streptomyces sp.; identical to SEQ ID NO: 145

SEQ ID NO: 216 - protein, 89 aa, Streptomyces sp.; identical to SEQ ID NO: 145

SEQ ID NO: 217 - DNA, 267 bp, Bos taurus
  source 1..267: mol_type = genomic DNA
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagct

SEQ ID NO: 218 - DNA, 267 bp, Sus scrofa; identical to SEQ ID NO: 217
  source 1..267: mol_type = genomic DNA

SEQ ID NO: 219 - DNA, 267 bp, Sus scrofa; identical to SEQ ID NO: 217
  source 1..267: mol_type = genomic DNA

SEQ ID NO: 220 - DNA, 267 bp, Gadus morhua; identical to SEQ ID NO: 217
  source 1..267: mol_type = genomic DNA

SEQ ID NO: 221 - DNA, 267 bp, Gadus morhua; identical to SEQ ID NO: 217
  source 1..267: mol_type = genomic DNA

SEQ ID NO: 222 - DNA, 279 bp, Danio rerio
  source 1..279: mol_type = genomic DNA
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcttacgtagaattc

SEQ ID NO: 223 - DNA, 267 bp, Beauveria bassiana; identical to SEQ ID NO: 217
  source 1..267: mol_type = genomic DNA

SEQ ID NO: 224 - skipped

SEQ ID NO: 225 - DNA, 267 bp, Macrovipera lebetinus; identical to SEQ ID NO: 217
  source 1..267: mol_type = genomic DNA

SEQ ID NO: 226 - DNA, 267 bp, Macrovipera lebetinus; identical to SEQ ID NO: 217
  source 1..267: mol_type = genomic DNA

SEQ ID NO: 227 - skipped

SEQ ID NO: 228 - DNA, 267 bp, Macrovipera lebetinus; identical to SEQ ID NO: 217
  source 1..267: mol_type = genomic DNA

SEQ ID NO: 229 - DNA, 267 bp, Macrovipera lebetinus; identical to SEQ ID NO: 217
  source 1..267: mol_type = genomic DNA

SEQ ID NO: 230 - DNA, 276 bp, Capra hircus
  source 1..276: mol_type = genomic DNA
atgaggcaggtttggttctcttggattgtgggattgttcctatgttttttcaacgtgtcttctgctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagct

SEQ ID NO: 231 - DNA, 276 bp, Rattus norvegicus; identical to SEQ ID NO: 230
  source 1..276: mol_type = genomic DNA

SEQ ID NO: 232 - DNA, 276 bp, Oryctolagus cuniculus; identical to SEQ ID NO: 230
  source 1..276: mol_type = genomic DNA

SEQ ID NO: 233 - DNA, 276 bp, Balaenoptera acutorostrata; identical to SEQ ID NO: 230
  source 1..276: mol_type = genomic DNA; sub_species = scammoni

SEQ ID NO: 234 - DNA, 276 bp, Gallus gallus; identical to SEQ ID NO: 230
  source 1..276: mol_type = genomic DNA

SEQ ID NO: 235 - DNA, 276 bp, Crocodylus porosus; identical to SEQ ID NO: 230
  source 1..276: mol_type = genomic DNA

SEQ ID NO: 236 - DNA, 276 bp, Bombus vosnesenskii; identical to SEQ ID NO: 230
  source 1..276: mol_type = genomic DNA

SEQ ID NO: 237 - DNA, 276 bp, Apis cerana; identical to SEQ ID NO: 230
  source 1..276: mol_type = genomic DNA

SEQ ID NO: 238 - DNA, 276 bp, Solenopsis invicta; identical to SEQ ID NO: 230
  source 1..276: mol_type = genomic DNA

SEQ ID NO: 239 - DNA, 276 bp, Danaus plexippus; identical to SEQ ID NO: 230
  source 1..276: mol_type = genomic DNA

SEQ ID NO: 240 - DNA, 276 bp, Photinus pyralis; identical to SEQ ID NO: 230
  source 1..276: mol_type = genomic DNA

SEQ ID NO: 241 - DNA, 276 bp, Aedes aegypti; identical to SEQ ID NO: 230
  source 1..276: mol_type = genomic DNA

SEQ ID NO: 242 - DNA, 276 bp, Tenebrio molitor; identical to SEQ ID NO: 230
  source 1..276: mol_type = genomic DNA

SEQ ID NO: 243 - DNA, 276 bp, Tenebrio molitor; identical to SEQ ID NO: 230
  source 1..276: mol_type = genomic DNA

SEQ ID NO: 244 - DNA, 276 bp, Tenebrio molitor; identical to SEQ ID NO: 230
  source 1..276: mol_type = genomic DNA

SEQ ID NO: 245 - DNA, 276 bp, Diatraea saccharalis; identical to SEQ ID NO: 230
  source 1..276: mol_type = genomic DNA

SEQ ID NO: 246 - DNA, 267 bp, Apis mellifera; identical to SEQ ID NO: 217
  source 1..267: mol_type = genomic DNA

SEQ ID NO: 247 - DNA, 267 bp, Apis mellifera; identical to SEQ ID NO: 217
  source 1..267: mol_type = genomic DNA

SEQ ID NO: 248 - DNA, 267 bp, Apis mellifera; identical to SEQ ID NO: 217
  source 1..267: mol_type = genomic DNA

SEQ ID NO: 249 - skipped

SEQ ID NO: 250 - DNA, 267 bp, Apis mellifera; identical to SEQ ID NO: 217
  source 1..267: mol_type = genomic DNA

SEQ ID NO: 251 - DNA, 267 bp, Apis mellifera; identical to SEQ ID NO: 217
  source 1..267: mol_type = genomic DNA

SEQ ID NO: 252 - DNA, 267 bp, Apis mellifera; identical to SEQ ID NO: 217
  source 1..267: mol_type = genomic DNA

SEQ ID NO: 253 - DNA, 267 bp, Apis mellifera; identical to SEQ ID NO: 217
  source 1..267: mol_type = genomic DNA

SEQ ID NO: 254-255 - skipped (2 entries)

SEQ ID NO: 256 - DNA, 267 bp, Apis mellifera; identical to SEQ ID NO: 217
  source 1..267: mol_type = genomic DNA

SEQ ID NO: 257 - DNA, 267 bp, Apis mellifera; identical to SEQ ID NO: 217
  source 1..267: mol_type = genomic DNA

SEQ ID NO: 258 - DNA, 267 bp, Apis mellifera; identical to SEQ ID NO: 217
  source 1..267: mol_type = genomic DNA

SEQ ID NO: 259 - DNA, 267 bp, Apis mellifera; identical to SEQ ID NO: 217
  source 1..267: mol_type = genomic DNA

SEQ ID NO: 260 - DNA, 267 bp, Apis mellifera; identical to SEQ ID NO: 217
  source 1..267: mol_type = genomic DNA

SEQ ID NO: 261 - DNA, 267 bp, Apis mellifera; identical to SEQ ID NO: 217
  source 1..267: mol_type = genomic DNA

SEQ ID NO: 262 - DNA, 267 bp, Apis mellifera; identical to SEQ ID NO: 217
  source 1..267: mol_type = genomic DNA

SEQ ID NO: 263 - DNA, 267 bp, Apis mellifera; identical to SEQ ID NO: 217
  source 1..267: mol_type = genomic DNA

SEQ ID NO: 264 - DNA, 267 bp, Apis mellifera; identical to SEQ ID NO: 217
  source 1..267: mol_type = genomic DNA

SEQ ID NO: 265 - DNA, 267 bp, Apis mellifera; identical to SEQ ID NO: 217
  source 1..267: mol_type = genomic DNA

SEQ ID NO: 266 - DNA, 267 bp, Apis mellifera; identical to SEQ ID NO: 217
  source 1..267: mol_type = genomic DNA

SEQ ID NO: 267 - DNA, 267 bp, Apis mellifera; identical to SEQ ID NO: 217
  source 1..267: mol_type = genomic DNA

SEQ ID NO: 268 - DNA, 267 bp, Apis mellifera; identical to SEQ ID NO: 217
  source 1..267: mol_type = genomic DNA

SEQ ID NO: 269 - DNA, 267 bp, Apis mellifera; identical to SEQ ID NO: 217
  source 1..267: mol_type = genomic DNA

SEQ ID NO: 270 - DNA, 267 bp, Apis mellifera; identical to SEQ ID NO: 217
  source 1..267: mol_type = genomic DNA

SEQ ID NO: 271 - DNA, 267 bp, Apis mellifera; identical to SEQ ID NO: 217
  source 1..267: mol_type = genomic DNA

SEQ ID NO: 272 - DNA, 267 bp, Apis mellifera; identical to SEQ ID NO: 217
  source 1..267: mol_type = genomic DNA

SEQ ID NO: 273 - skipped

SEQ ID NO: 274 - DNA, 267 bp, Apis mellifera; identical to SEQ ID NO: 217
  source 1..267: mol_type = genomic DNA

SEQ ID NO: 275 - DNA, 267 bp, Apis mellifera; identical to SEQ ID NO: 217
  source 1..267: mol_type = genomic DNA

SEQ ID NO: 276 - DNA, 267 bp, Apis mellifera; identical to SEQ ID NO: 217
  source 1..267: mol_type = genomic DNA

SEQ ID NO: 277 - DNA, 267 bp, Apis mellifera; identical to SEQ ID NO: 217
  source 1..267: mol_type = genomic DNA

SEQ ID NO: 278 - DNA, 267 bp, Apis mellifera; identical to SEQ ID NO: 217
  source 1..267: mol_type = genomic DNA

SEQ ID NO: 279 - DNA, 267 bp, Apis mellifera; identical to SEQ ID NO: 217
  source 1..267: mol_type = genomic DNA

SEQ ID NO: 280 - DNA, 267 bp, Apis mellifera; identical to SEQ ID NO: 217
  source 1..267: mol_type = genomic DNA

SEQ ID NO: 281 - DNA, 267 bp, Gammaproteobacteria bacterium; identical to SEQ ID NO: 217
  source 1..267: mol_type = genomic DNA

SEQ ID NO: 282 - DNA, 267 bp, Pseudoalteromonas sp.; identical to SEQ ID NO: 217
  source 1..267: mol_type = genomic DNA

SEQ ID NO: 283 - DNA, 267 bp, Scytalidium lignicola; identical to SEQ ID NO: 217
  source 1..267: mol_type = genomic DNA

SEQ ID NO: 284 - DNA, 267 bp, Metarhizium anisopliae; identical to SEQ ID NO: 217
  source 1..267: mol_type = genomic DNA

SEQ ID NO: 285 - DNA, 267 bp, Thermobifida fusca; identical to SEQ ID NO: 217
  source 1..267: mol_type = genomic DNA

SEQ ID NO: 286 - DNA, 267 bp, Streptomyces griseus; identical to SEQ ID NO: 217
  source 1..267: mol_type = genomic DNA

SEQ ID NO: 287 - DNA, 267 bp, Streptomyces sp.; identical to SEQ ID NO: 217
  source 1..267: mol_type = genomic DNA

SEQ ID NO: 288 - DNA, 267 bp, Streptomyces sp.; identical to SEQ ID NO: 217
  source 1..267: mol_type = genomic DNA

SEQ ID NO: 289 - protein, 238 aa, Daboia siamensis
QKSSELVVGGDECNINEHRSLVFLYNNSFGCSGTLINQQWVLSAVHCDMENVRIYLGVHNLTLRNNAEIRLPEERFFCLSNKNYTKWDKDIMLIKLDRPVKTSTYIAPLSLPSSPPRVGSVCRIMGWGAITSPNETFPGVTHCANINILPYSVCRAAYKGLPAQSRTLCGGILEGGIGSCMGDSGGPLICNGEMHGIVAWGDDTCAQPHKPVHYTKVYDYTDWIQSIIAGNTAATCPP

SEQ ID NO: 290 - protein, 219 aa, Beauveria bassiana
IAGGQDAELGWFPYTVSLRNRGRHNCGGIIIDQSSILTAAHCIVGLSADRVSIVAGAVKLDSGGTTYQVSAIHKHPGYNSSTITNDIAVLKLAKPLSYTPYIRPIALATQDPPAGSGTLGSGWGVSRSSGPVSNTLQWISLQSLDHKICQEKMGVTVSQICTTSANKGGMCRGDSGGPLTDERITLVGVYSWGRGCGDGYPDVYASVAYHREWIRSYMG

SEQ ID NO: 291 - DNA, 714 bp, Daboia siamensis
  source 1..714: mol_type = genomic DNA
caaaagtcttcagagcttgttgtcggcggtgacgagtgcaacatcaacgagcacagatcactggtgttcctctacaacaactctttcggatgttctggtactcttatcaaccaacaatgggtgctttccgcagtccactgtgatatggaaaacgttcgtatctacttgggtgtacacaatctaactctcaggaacaatgctgaaatcagattgccagaagagagattcttttgtctaagtaataagaattatactaaatgggacaaggacataatgctgattaagctggacagaccagttaaaacctccacttacatcgcccctctttccctcccctcttctccaccaagagtcggttctgtctgtagaattatgggttggggtgctattacgtctcctaacgaaacatttccaggcgttacccactgcgcaaacatcaacattctcccttactctgtttgtagagccgcctacaagggtttacctgctcagtccagaaccctatgtggaggtattctggaaggtggtattggttcctgtatgggagacagcggaggtcctctaatctgtaatggtgagatgcacggaatcgttgcttggggagacgacacttgcgctcaaccacataagccagttcattatactaaggtctacgattacactgattggattcaatcgattattgctggtaacaccgcagctacctgccctcca

SEQ ID NO: 292 - DNA, 657 bp, Beauveria bassiana
  source 1..657: mol_type = genomic DNA
attgcaggagggcaagatgcagaacttggctggtttccatacacggtctcgttaagaaatcgaggtagacataattgtggtgggattatcatagaccaatcatcaattctgactgcagcccactgtatagttggtctaagcgctgacagagtttctatcgtggctggtgctgtcaaattggacagtggaggtaccacttaccaagtatcagctattcacaagcaccctggatacaattcgtcgaccataacaaatgatatagctgtactgaagctcgcaaaaccgttgagttacacgccctatataaggcctatagccttggccactcaggatccccctgctggttcaggtactctgggttctggctggggggtgtcgagatcgtccggtcctgtctctaatacattacagtggatctctctgcaatcattggatcacaagatctgccaggagaaaatgggcgttactgtttcccagatctgcactactagcgctaacaaagggggtatgtgtcgtggggactctggtggtccgctaactgatgagagaatcactcttgtaggtgtgtacagttggggacgtggatgcggggacggatacccagacgtgtacgcctctgtggcatatcaccgagagtggatcagatcctatatggga

SEQ ID NO: 293 - protein, 6 aa, Synthetic construct
WIGHIY

SEQ ID NO: 294 - protein, 4 aa, Synthetic construct
WIGH

SEQ ID NO: 295 - skipped

SEQ ID NO: 296 - protein, 20 aa, Synthetic construct
FGGGTKVEIKRTVAAPSVFI

SEQ ID NO: 297 - protein, 22 aa, Synthetic construct
LAFGGGTKVEIKRTVAAPSVFI

SEQ ID NO: 298 - protein, 11 aa, Synthetic construct
FGGGTKVEIKR

(19) United States
(12) Patent Application Publication
(10) Pub. No.: US 2026/0125664 A1
(43) Pub. Date: May 7, 2026

(54) COMPOSITIONS AND METHODS RELATING TO RECOMBINANT CHYMOTRYPSIN

(71) Applicant: Promega Corporation, Madison, WI (US)

(72) Inventors: Alba Katiria GONZALEZ RIVERA, Madison, WI (US); Sergei SAVELIEV, Madison, WI (US); Marjeta URH, Madison, WI (US); Michael KILLORAN, Madison, WI (US); Lance ENCELL, Madison, WI (US)

(21) Appl. No.: 19/285,921

(22) Filed: Jul. 30, 2025

Related U.S. Application Data
(60) Provisional application No. 63/677,360, filed on Jul. 30, 2024.

(51) Int. Cl. C12N 9/64; C07K 14/435; C12N 15/81; C12R 1/84

(52) U.S. Cl. CPC C12N 9/6408; C07K 14/43572; C12N 15/815; C07K 2319/21; C12R 2001/84; C12Y 304/21001

(57) ABSTRACT

Provided herein are compositions, methods, and kits related to chymotrypsin production for characterizing proteins, assessing, and monitoring disease stages and phases, predicting the likelihood of disease progression, predicting and monitoring responses to disease therapies, and treating disease conditions.

### CROSS-REFERENCE

This application claims the benefit of U.S. Provisional Patent Application No. 63/677,360, filed Jul. 30, 2024; which is incorporated by reference herein in its entirety.

### SEQUENCE LISTING

The text of the computer readable sequence listing filed herewith, titled “PRMG_43026_202_SequenceListing_corrected.xml,” created Sep. 29, 2025, having a file size of 340,809 bytes, is hereby incorporated by reference in its entirety.

### FIELD

Provided herein are compositions, methods, and kits related to recombinant chymotrypsin production for protein analytical applications such as peptide mapping and proteomics and clinical use including treatment of wounds, ulcers, asthma, and other applications.

### BACKGROUND

Chymotrypsin can be used to characterize proteins. However, their use is limited due to their compromised cleavage specificity (e.g., chymotrypsins can cleave at aromatic residues tyrosine, phenylalanine, and tryptophan, but also cleave at other residues, which complicates protein analysis), protein analysis reproducibility (e.g., reproducibility of protein analysis using chymotrypsin is adversely affected by the age and health of the animal from which it is extracted), and performance (e.g., performance is compromised by contamination with proteases that are co-extracted with chymotrypsin from pancreas). Thus, compositions and methods are needed for chymotrypsin production with improved cleavage specificity, high lot-to-lot reproducibility and straightforward enzyme purification and improved purity.

### SUMMARY

Provided herein are compositions, methods, and kits related to chymotrypsin production for protein analytical applications such as peptide mapping and proteomics and clinical use including treatment of wounds, ulcers, asthma, and other applications.

Embodiments of the present disclosure include compositions comprising an exogenous nucleic acid sequence encoding a recombinant chymotrypsin polypeptide. In some embodiments, the nucleic acid sequence is stably integrated into the genome of a cell. In some embodiments, the nucleic acid sequence is transiently expressed in a cell. In some embodiments, the cell is a yeast cell. In some embodiments, the cell is Pichia pastoris. In some embodiments, the recombinant chymotrypsin polypeptide comprises any one or more of a secretion signal, a pro-peptide, or a purification tag. In some embodiments, the exogenous nucleic acid sequence comprises a promoter and/or encodes any one or more of a secretion signal, a pro-peptide, or a purification tag.

Embodiments of the present disclosure include compositions comprising a recombinant chymotrypsin polypeptide. In some embodiments, the recombinant chymotrypsin polypeptide is expressed from a nucleic acid sequence that is stably or transiently integrated into the genome of a cell. In some embodiments, the cell is a yeast cell. In some embodiments, the cell is Pichia pastoris. In some embodiments, the recombinant chymotrypsin polypeptide comprises any one or more of a secretion signal, a pro-peptide, or a purification tag.

In some embodiments, the recombinant chymotrypsin polypeptide and/or polynucleotide encoding a c recombinant chymotrypsin polypeptide comprises a sequence of any one of a Bovine (Bos taurus) chymotrypsin, a Porcine (Sus scrofa) chymotrypsin B, a Porcine (Sus scrofa) chymotrypsin variant N127Q, a Cod (Gadus morhua) chymotrypsin A, a Cod (Gadus morhua) chymotrypsin B, a Zebrafish (Danio rerio) chymotrypsin, a Beauveria bassiana chymotrypsin variant S81L, a Snake (Daboia siamensis) chymotrypsin E5L0E4 variant T62R/N81S/N83S/N134Q, a Snake (Macrovipera lebetinus) chymotrypsin VLCTLP, a Snake (Macrovipera lebetinus) chymotrypsin VLCTLP variant N26Q/N82Q/N98Q/N135Q/N232Q, a Snake (Macrovipera lebetinus) chymotrypsin VLCTLP variant N26Q/N82Q/N98Q/N135Q/N232A, a Snake (Macrovipera lebetinus) chymotrypsin VLAF, a Snake (Macrovipera lebetinus) chymotrypsin VLAF variant N26Q/N61Q/N83Q, a Goat (Capra hircus) chymotrypsin, a Rat (Rattus norvegicus) chymotrypsin, a Rabbit (Oryctolagus cuniculus) chymotrypsin, a Whale (Balaenoptera acutorostrata scammoni) chymotrypsin, a Chicken (Gallus gallus) chymotrypsin, a Crocodile (Crocodylus porosus) chymotrypsin, a Bumble bee (Bombus vosnesenskii) chymotrypsin, a Asian honeybee (Apis cerena cerena) chymotrypsin, an Ant (Solenopsis invicta) chymotrypsin, a Butterfly (Danaus plexippus Plexippus) chymotrypsin, a Firefly (Photinus pyralis) chymotrypsin, a Mosquito (Aedes aegypti) chymotrypsin, a Beetle (Tenebrio molitor) chymotrypsin A0A8J6LPH0, a Beetle (Tenebrio molitor) chymotrypsin A0A8J6HC31, a Beetle (Tenebrio molitor) chymotrypsin A0A8J6GYN2, a Moth (Diatraea saccharalis) chymotrypsin A0A9N9R2F0, a Honeybee (Apis mellifera) chymotrypsin L3W4, a Honeybee (Apis mellifera) chymotrypsin R3J2, a Honeybee (Apis mellifera) chymotrypsin, a Honeybee (Apis mellifera) chymotrypsin variant A180S/V191C/P204DI/D211V, a Honeybee (Apis mellifera) chymotrypsin variant C127A, a Honeybee (Apis mellifera) chymotrypsin variant C127A/1128C/V190C, a Honeybee (Apis mellifera) chymotrypsin variant K29G/Q62V/R84P/V191C, a Honeybee (Apis mellifera) chymotrypsin variant N28Q/K29G/Q62V/R84P/V191C, a Honeybee (Apis mellifera) chymotrypsin variant A180P, a Honeybee (Apis mellifera) chymotrypsin variant A180V, a Honeybee (Apis mellifera) chymotrypsin variant D193GP, a Honeybee (Apis mellifera) chymotrypsin variant D211G, a Honeybee (Apis mellifera) chymotrypsin variant D211H, a Honeybee (Apis mellifera) chymotrypsin variant E153I, a Honeybee (Apis mellifera) chymotrypsin variant F173L, a Honeybee (Apis mellifera) chymotrypsin variant F201W, a Honeybee (Apis mellifera) chymotrypsin variant F229A/L230N/A231-245, a Honeybee (Apis mellifera) chymotrypsin variant F242A/Y243R, a Honeybee (Apis mellifera) chymotrypsin variant G1771, a Honeybee (Apis mellifera) chymotrypsin variant G179S/A180S/V191C/P204ST/D211V, a Honeybee (Apis mellifera) chymotrypsin variant K7Y, a Honeybee (Apis mellifera) chymotrypsin variant K7Y/F242A/Y243R, a Honeybee (Apis mellifera) chymotrypsin variant K7Y/S228A/F229N/A231-245, a Honeybee (Apis mellifera) chymotrypsin variant K7Y/V191C, a Honeybee (Apis mellifera) chymotrypsin variant K7Y/V191C/4229-245, a Honeybee (Apis mellifera) chymotrypsin variant N28Q, a Honeybee (Apis mellifera) chymotrypsin variant P204A, a Honeybee (Apis mellifera) chymotrypsin variant P204C/C205P, a Honeybee (Apis mellifera) chymotrypsin variant V191C, a Honeybee (Apis mellifera) chymotrypsin variant V191C/4229-245, a Honeybee (Apis mellifera) chymotrypsin variant V199G, a Honeybee (Apis mellifera) chymotrypsin variant V199W, a Honeybee (Apis mellifera) chymotrypsin variant 4229-245, a Gammaproteobacteria bacterium chymotrypsin-like protease, a Pseudoalteromonas sp. SM1988 chymotrypsin-like protease, a Scytalidoglutamic (Scytalidium lignicola) peptidase, a Metarhizium anisopliae chymotrypsinogen-like protease, a T. fusca chymotrypsinogen-like protease, a Streptomyces griseus Protease C (chymotrypsin-like protease), a Streptomyces sp. chymotrypsinogen-like protease, a Streptomyces sp. chymotrypsinogen-like protease propeptide variant, a Daboia siamensis recombinant chymotrypsin E5L0E4 wild-type, or a Beauveria bassiana chymotrypsin wild-type.

In some embodiments, any one of the recombinant chymotrypsin polypeptides comprise at least 70% (e.g., 70%, 759%, 80%, 90%, 959%, 99%, 100%, or ranges therebetween) sequence identity to amino acid sequence SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70; SEQ ID NO: 71; SEQ ID NO: 72; SEQ ID NO: 289; or SEQ ID NO: 290. In some embodiments, recombinant chymotrypsin polypeptide further comprises a purification tag. In some embodiments, the purification tag is a C-terminal histidine tag.

In some embodiments, the nucleic acid (encoding a recombinant chymotrypsin polypeptide) comprises at least 70% (e.g., 70%, 75%, 80%, 90%, 95%, 99%, 100%, or ranges therebetween) sequence identity to SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO:100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144; SEQ ID NO: 291; or SEQ ID NO: 292.

In some embodiments herein, a recombinant chymotrypsin is a honeybee recombinant chymotrypsin L3W4 and comprises at least 70% (e.g., 70%, 75%, 80%, 90%, 95%, 99%, 100%, or ranges therebetween) sequence identity to SEQ ID NO: 30 and/or is encoded by a nucleic acid having at least 70% (e.g., 70%, 75%, 80%, 90%, 95%, 99%, 100%, or ranges therebetween) sequence identity to SEQ ID NO: 102.

In some embodiments herein, a recombinant chymotrypsin is a honeybee recombinant chymotrypsin R3J2 and comprises at least 70% (e.g., 70%, 75%, 80%, 85%, 90%, 95%, 100%, or ranges therebetween) sequence identity to SEQ ID NO: 31 and/or is encoded by a nucleic acid having at least 70% (e.g., 70%, 75%, 80%, 85%, 90%, 95%, 100%, or ranges therebetween) sequence identity to SEQ ID NO: 103.

In some embodiments herein, a recombinant chymotrypsin is a honeybee recombinant chymotrypsin and comprises at least 70% (e.g., 70%, 75%, 80%, 85%, 90%, 95%, 100%, or ranges therebetween) sequence identity to SEQ ID NO: 32 and/or is encoded by a nucleic acid having at least 70% (e.g., 70%, 75%, 80%, 85%, 90%, 95%, 100%, or ranges therebetween) sequence identity to SEQ ID NO: 104.

In some embodiments herein, a recombinant chymotrypsin is a honeybee recombinant chymotrypsin and comprises at least 70% (e.g., 70%, 75%, 80%, 85%, 90%, 95%, 100%, or ranges therebetween) sequence identity to SEQ ID NO: 33 and/or is encoded by a nucleic acid having at least 70% (e.g., 70%, 75%, 80%, 85%, 90%, 95%, 100%, or ranges therebetween) sequence identity to SEQ ID NO: 105.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 34 and/or is encoded by a nucleic acid having at least 70% (e.g., 70%, 75%, 80%, 85%, 90%, 95%, 100%, or ranges therebetween) sequence identity to SEQ ID NO: 106.

In some embodiments, the mutant honeybee recombinant chymotrypsin comprises one or more of a S at position 180, a C at position 191, a DI at position 204, and a V at position 211 relative to SEQ ID NO: 34.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 35 and/or is encoded by a nucleic acid having at least 70% (e.g., 70%, 75%, 80%, 85%, 90%, 95%, 100%, or ranges therebetween) sequence identity to SEQ ID NO: 107. In some embodiments, the mutant honeybee recombinant chymotrypsin comprises an A at position 127 relative to SEQ ID NO: 35.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 36 and/or is encoded by a nucleic acid having at least 70% (e.g., 70%, 75%, 80%, 85%, 90%, 95%, 100%, or ranges therebetween) sequence identity to SEQ ID NO: 108. In some embodiments, the mutant honeybee recombinant chymotrypsin comprises an A at position 127, a C at position 128, and a C at position 190 relative to SEQ ID NO: 36.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 37 and/or is encoded by a nucleic acid having at least 70% (e.g., 70%, 75%, 80%, 85%, 90%, 95%, 100%, or ranges therebetween) sequence identity to SEQ ID NO:109. In some embodiments, the mutant honeybee recombinant chymotrypsin comprises a G at position 29, a V at position 62, a P at position 84, and a C at position 191 relative to SEQ ID NO: 37.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 38 and/or is encoded by a nucleic acid having at least 70% (e.g., 70%, 75%, 80%, 85%, 90%, 95%, 100%, or ranges therebetween) sequence identity to SEQ ID NO: 110. In some embodiments, the mutant honeybee recombinant chymotrypsin comprises one or more of a G at position 29, a V at position 62, a P at position 84, and a C at position 191 relative to SEQ ID NO: 38.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 39 and/or is encoded by a nucleic acid having at least 70% (e.g., 70%, 75%, 80%, 85%, 90%, 95%, 100%, or ranges therebetween) sequence identity to SEQ ID NO: 111. In some embodiments, the mutant honeybee recombinant chymotrypsin comprises a Q at position 28, a G at position 29, a V at position 62, a P at position 84, and a C at position 191 relative to SEQ ID NO: 39.

In some embodiments herein, a recombinant chymotrypsin is a mutant honeybee recombinant chymotrypsin D211G and comprises at least 70% (e.g., 70%, 75%, 80%, 85%, 90%, 95%, 100%, or ranges therebetween) sequence identity to SEQ ID NO:43 and/or is encoded by a nucleic acid having at least 70% (e.g., 70%, 75%, 80%, 85%, 90%, 95%, 100%, or ranges therebetween) sequence identity to SEQ ID NO: 115. In some embodiments, the mutant honeybee recombinant chymotrypsin comprises a G at position 211 relative to SEQ ID NO: 43.

In some embodiments herein, a recombinant chymotrypsin is a mutant honeybee recombinant chymotrypsin D211H and comprises at least 70% (e.g., 70%, 75%, 80%, 85%, 90%, 95%, 100%, or ranges therebetween) sequence identity to SEQ ID NO: 44 and/or is encoded by a nucleic acid having at least 70% (e.g., 70%, 75%, 80%, 85%, 90%, 95%, 100%, or ranges therebetween) sequence identity to SEQ ID NO: 116. In some embodiments, the mutant honeybee recombinant chymotrypsin comprises an H at position 211 relative to SEQ ID NO: 44.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 40 and/or is encoded by a nucleic acid having at least 70% (e.g., 70%, 75%, 80%, 85%, 90%, 95%, 100%, or ranges therebetween) sequence identity to SEQ ID NO: 112. In some embodiments, the mutant honeybee recombinant chymotrypsin comprises a P at position 180 relative to SEQ ID NO: 40.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 41 and/or is encoded by a nucleic acid having at least 70% (e.g., 70%, 75%, 80%, 85%, 90%, 95%, 100%, or ranges therebetween) sequence identity to SEQ ID NO: 113. In some embodiments, the mutant honeybee recombinant chymotrypsin comprises a V at position 180 relative to SEQ ID NO: 41.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 42 and/or is encoded by a nucleic acid having at least 70% (e.g., 70%, 75%, 80%, 85%, 90%, 95%, 100%, or ranges therebetween) sequence identity to SEQ ID NO: 114. In some embodiments, the mutant honeybee recombinant chymotrypsin comprises a GP at position 193 relative to SEQ ID NO: 42.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 45 and/or is encoded by a nucleic acid having at least 70% (e.g., 70%, 75%, 80%, 85%, 90%, 95%, 100%, or ranges therebetween) sequence identity to SEQ ID NO: 117. In some embodiments, the mutant honeybee recombinant chymotrypsin comprises one or more of an I at position 153 relative to SEQ ID NO: 45.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 46 and/or is encoded by a nucleic acid having at least 70% (e.g., 70%, 75%, 80%, 85%, 90%, 95%, 100%, or ranges therebetween) sequence identity to SEQ ID NO: 118. In some embodiments, the mutant honeybee recombinant chymotrypsin comprises a L at position 173 relative to SEQ ID NO: 46.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 47 and/or is encoded by a nucleic acid having at least 70% (e.g., 70%, 75%, 80%, 85%, 90%, 95%, 100%, or ranges therebetween) sequence identity to SEQ ID NO: 119. In some embodiments, the mutant honeybee recombinant chymotrypsin comprises one or more of a W at position 201 relative to SEQ ID NO: 47.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 48 and/or is encoded by a nucleic acid having at least 70% (e.g., 70%, 75%, 80%, 85%, 90%, 95%, 100%, or ranges therebetween) sequence identity to SEQ ID NO: 120. In some embodiments, the mutant honeybee recombinant chymotrypsin comprises one or more of an A at position 229, a N at position 230, or a deletion of positions 231-245 relative to SEQ ID NO: 48.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 49 and/or is encoded by a nucleic acid having at least 70% (e.g., 70%, 75%, 80%, 85%, 90%, 95%, 100%, or ranges therebetween) sequence identity to SEQ ID NO: 121. In some embodiments, the mutant honeybee recombinant chymotrypsin comprises one or more of an A at position 242, or a R at position 243 relative to SEQ ID NO: 49.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 50 and/or is encoded by a nucleic acid having at least 70% (e.g., 70%, 75%, 80%, 85%, 90%, 95%, 100%, or ranges therebetween) sequence identity to SEQ ID NO: 122. In some embodiments, the mutant honeybee recombinant chymotrypsin comprises an I at position 177 relative to SEQ ID NO: 50.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 51 and/or is encoded by a nucleic acid having at least 70% (e.g., 70%, 75%, 80%, 85%, 90%, 95%, 100%, or ranges therebetween) sequence identity to SEQ ID NO: 123. In some embodiments, the mutant honeybee recombinant chymotrypsin comprises one or more of a S at position 179, an S at position 180, a C at position 191, a ST at position 204, or a V at position 211 relative to SEQ ID NO: 51.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 52 and/or is encoded by a nucleic acid having at least 70% (e.g., 70%, 75%, 80%, 85%, 90%, 95%, 100%, or ranges therebetween) sequence identity to SEQ ID NO: 124. In some embodiments, the mutant honeybee recombinant chymotrypsin comprises a Y at position 7 relative to SEQ ID NO: 52.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 53 and/or is encoded by a nucleic acid having at least 70% (e.g., 70%, 75%, 80%, 85%, 90%, 95%, 100%, or ranges therebetween) sequence identity to SEQ ID NO: 125. In some embodiments, the mutant honeybee recombinant chymotrypsin comprises one or more of a Y at position 7, an A at position 242, or an R at position 243 relative to SEQ ID NO: 53.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 54 and/or is encoded by a nucleic acid having at least 70% (e.g., 70%, 75%, 80%, 85%, 90%, 95%, 100%, or ranges therebetween) sequence identity to SEQ ID NO: 126. In some embodiments, the mutant honeybee recombinant chymotrypsin comprises a Y at position 7, an A at position 228, an N at position 229, or a deletion of positions 231-245 relative to SEQ ID NO: 54.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 55 and/or is encoded by a nucleic acid having at least 70% (e.g., 70%, 75%, 80%, 85%, 90%, 95%, 100%, or ranges therebetween) sequence identity to SEQ ID NO: 127. In some embodiments, the mutant honeybee recombinant chymotrypsin comprises a Y at position 7 or a C at position 191 relative to SEQ ID NO: 55.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 56 and/or is encoded by a nucleic acid having at least 70% (e.g., 70%, 75%, 80%, 85%, 90%, 95%, 100%, or ranges therebetween) sequence identity to SEQ ID NO: 128. In some embodiments, the mutant honeybee recombinant chymotrypsin comprises one or more of a Y at position 7, a C at position 191, or a deletion of positions 229-245 relative to SEQ ID NO: 56.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 57 and/or is encoded by a nucleic acid having at least 70% (e.g., 70%, 75%, 80%, 85%, 90%, 95%, 100%, or ranges therebetween) sequence identity to SEQ ID NO: 129. In some embodiments, the mutant honeybee recombinant chymotrypsin comprises one or more of a Q at position 28 relative to SEQ ID NO: 57.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 58 and/or is encoded by a nucleic acid having at least 70% (e.g., 70%, 75%, 80%, 85%, 90%, 95%, 100%, or ranges therebetween) sequence identity to SEQ ID NO: 130. In some embodiments, the mutant honeybee recombinant chymotrypsin comprises one or more of an A at position 204 relative to SEQ ID NO: 58.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 59 and/or is encoded by a nucleic acid having at least 70% (e.g., 70%, 75%, 80%, 85%, 90%, 95%, 100%, or ranges therebetween) sequence identity to SEQ ID NO: 131. In some embodiments, the mutant honeybee recombinant chymotrypsin comprises one or more of a C at position 204 or a P at position 205 relative to SEQ ID NO: 59.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 60 and/or is encoded by a nucleic acid having at least 70% (e.g., 70%, 75%, 80%, 85%, 90%, 95%, 100%, or ranges therebetween) sequence identity to SEQ ID NO: 132. In some embodiments, the mutant honeybee recombinant chymotrypsin comprises one or more of a V at position 191 relative to SEQ ID NO: 60.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 61 and/or is encoded by a nucleic acid having at least 70% (e.g., 70%, 75%, 80%, 85%, 90%, 95%, 100%, or ranges therebetween) sequence identity to SEQ ID NO: 133. In some embodiments, the mutant honeybee recombinant chymotrypsin comprises one or more of a V at position 191 or a deletion of positions 229-245 relative to SEQ ID NO: 61.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 62 and/or is encoded by a nucleic acid having at least 70% (e.g., 70%, 75%, 80%, 85%, 90%, 95%, 100%, or ranges therebetween) sequence identity to SEQ ID NO: 134. In some embodiments, the mutant honeybee recombinant chymotrypsin comprises one or more of a G at position 199 relative to SEQ ID NO: 62.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 63 and/or is encoded by a nucleic acid having at least 70% (e.g., 70%, 75%, 80%, 85%, 90%, 95%, 100%, or ranges therebetween) sequence identity to SEQ ID NO: 135. In some embodiments, the mutant honeybee recombinant chymotrypsin comprises one or more of a W at position 199 relative to SEQ ID NO: 63.

In some embodiments herein, a recombinant chymotrypsin is a mutant honeybee recombinant chymotrypsin and comprises at least 70% (e.g., 70%, 75%, 80%, 85%, 90%, 95%, 100%, or ranges therebetween) sequence identity to SEQ ID NO: 64 and/or is encoded by a nucleic acid having at least 70% (e.g., 70%, 75%, 80%, 85%, 90%, 95%, 100%, or ranges therebetween) sequence identity to SEQ ID NO: 136. In some embodiments, the mutant honeybee recombinant chymotrypsin comprises one or more of a deletion of positions 229-245 relative to SEQ ID NO: 64.

In some embodiments herein, a recombinant chymotrypsin is a bovine recombinant chymotrypsin and comprises at least 70% (e.g., 70%, 75%, 80%, 85%, 90%, 95%, 100%, or ranges therebetween) sequence identity to SEQ ID NO: 1 and/or is encoded by a nucleic acid having at least 70% (e.g., 70%, 75%, 80%, 85%, 90%, 95%, 100%, or ranges therebetween) sequence identity to SEQ ID NO: 73. In some embodiments, the recombinant chymotrypsin is a porcine recombinant chymotrypsin and comprises at least 70% (e.g., 70%, 75%, 80%, 85%, 90%, 95%, 100%, or ranges therebetween) sequence identity to SEQ ID NO: 2 and/or is encoded by a nucleic acid having at least 70% (e.g., 70%, 75%, 80%, 85%, 90%, 95%, 100%, or ranges therebetween) sequence identity to SEQ ID NO: 74.

In some embodiments herein, a recombinant chymotrypsin is a mutant porcine recombinant chymotrypsin and comprises at least 70% (e.g. 70%, 75%, 80%, 85%, 90%, 95%, 100%, or ranges therebetween) sequence identity to SEQ ID NO: 3 and/or is encoded by a nucleic acid having at least 70% (e.g., 70%, 75%, 80%, 85%, 90%, 95%, 100%, or ranges therebetween) sequence identity to SEQ ID NO:75. In some embodiments, the mutant porcine recombinant chymotrypsin comprises a Q at position 127 relative to SEQ ID NO: 3.

In some embodiments herein, a recombinant chymotrypsin is a cod recombinant chymotrypsin and comprises at least 70% (e.g., 70%, 75%, 80%, 85%, 90%, 95%, 100%, or ranges therebetween) sequence identity to SEQ ID NO: 4 and/or is encoded by a nucleic acid having at least 70% (e.g., 70%, 75%, 80%, 85%, 90%, 95%, 100%, or ranges therebetween) sequence identity to SEQ ID NO: 76. In some embodiments, the recombinant chymotrypsin is a cod recombinant chymotrypsin and comprises at least 70% (e.g., 70%, 75%, 80%, 85%, 90%, 95%, 100%, or ranges therebetween) sequence identity to SEQ ID NO: 5 and/or is encoded by a nucleic acid having at least 70% (e.g., 70%, 75%, 80%, 85%, 90%, 95%, 100%, or ranges therebetween) sequence identity to SEQ ID NO: 77.

In some embodiments herein, a recombinant chymotrypsin is a zebrafish recombinant chymotrypsin and comprises at least 70% (e.g., 70%, 75%, 80%, 85%, 90%, 95%, 100%, or ranges therebetween) sequence identity to SEQ ID NO: 6 and/or is encoded by a nucleic acid having at least 70% (e.g., 70%, 75%, 80%, 85%, 90%, 95%, 100%, or ranges therebetween) sequence identity to SEQ ID NO: 78. In some embodiments, the recombinant chymotrypsin is a Macrovipera lebetinus (VLCTLP) recombinant chymotrypsin and comprises at least 70% (e.g., 70%, 75%, 80%, 85%, 90%, 95%, 100%, or ranges therebetween) sequence identity to SEQ ID NO: 9 and/or is encoded by a nucleic acid having at least 70% (e.g., 70%, 75%, 80%, 85%, 90%, 95%, 100%, or ranges therebetween) sequence identity to SEQ ID NO: 81.

In some embodiments herein, a recombinant chymotrypsin is a mutant Macrovipera lebetinus (VLCTLP) recombinant chymotrypsin and comprises at least 70% (e.g., 70%, 75%, 80%, 85%, 90%, 95%, 100%, or ranges therebetween) sequence identity to SEQ ID NO: 10 and/or is encoded by a nucleic acid having at least 70% (e.g., 70%, 75%, 80%, 85%, 90%, 95%, 100%, or ranges therebetween) sequence identity to SEQ ID NO: 82. In some embodiments, the recombinant chymotrypsin comprises one or more of a Q at position 26, a Q at position 82, a Q at position 98, a Q at position 135, or a Q at position 232 relative to SEQ ID NO: 10.

In some embodiments herein, a recombinant chymotrypsin is a mutant Macrovipera lebetinus (VLCTLP) recombinant chymotrypsin and comprises at least 70% (e.g., 70%, 75%, 80%, 85%, 90%, 95%, 100%, or ranges therebetween) sequence identity to SEQ ID NO: 11 and/or is encoded by a nucleic acid having at least 70% (e.g., 70%, 75%, 80%, 85%, 90%, 95%, 100%, or ranges therebetween) sequence identity to SEQ ID NO: 83. In some embodiments, the recombinant chymotrypsin comprises one or more of a Q at position 26, a Q at position 82, a Q at position 98, a Q at position 135, or an A at position 232 relative to SEQ ID NO: 11.

In some embodiments herein, a recombinant chymotrypsin is a mutant Daboia siamensis recombinant chymotrypsin E5L0E4 and comprises at least 70% (e.g., 70%, 75%, 80%, 85%, 90%, 95%, 100%, or ranges therebetween) sequence identity to SEQ ID NO: 8 and/or is encoded by a nucleic acid having at least 70% (e.g., 70%, 75%, 80%, 85%, 90%, 95%, 100%, or ranges therebetween) sequence identity to SEQ ID NO: 80. In some embodiments, the recombinant chymotrypsin comprises one or more of a R at position 62, a S at position 81, a S at position 83, or a Q at position 134 relative to SEQ ID NO: 8.

In some embodiments herein, a recombinant chymotrypsin is a Beauveria bassiana recombinant chymotrypsin N81L and comprises at least 70% (e.g., 70%, 75%, 80%, 85%, 90%, 95%, 100%, or ranges therebetween) sequence identity to SEQ ID NO: 7 and/or is encoded by a nucleic acid having at least 70% (e.g., 70%, 75%, 80%, 85%, 90%, 95%, 100%, or ranges therebetween) sequence identity to SEQ ID NO: 79.

In some embodiments, the recombinant chymotrypsin comprises a L at position 81 relative to SEQ ID NO: 7.

In some embodiments herein, a recombinant chymotrypsin is a snake recombinant chymotrypsin and comprises at least 70% (e.g., 70%, 75%, 80%, 85%, 90%, 95%, 100%, or ranges therebetween) sequence identity to any one of SEQ ID NO: 8, SEQ ID NO: 9, SEQ ID NO: 10, SEQ ID NO: 11, SEQ ID NO: 12, or SEQ ID NO: 13, and/or is encoded by a nucleic acid having at least 70% (e.g., 70%, 75%, 80%, 85%, 90%, 95%, 100%, or ranges therebetween) sequence identity to SEQ ID NO: 80, SEQ ID NO: 81, SEQ ID NO: 82, SEQ ID NO: 83, SEQ ID NO: 84, or SEQ ID NO: 85.

In some embodiments herein, a recombinant chymotrypsin is a Macrovipera lebetinus chymotrypsin VLAF and comprises at least 70% (e.g., 70%, 75%, 80%, 85%, 90%, 95%, 100%, or ranges therebetween) sequence identity to SEQ ID NO: 12 and/or is encoded by a nucleic acid having at least 70% (e.g., 70%, 75%, 80%, 85%, 90%, 95%, 100%, or ranges therebetween) sequence identity to SEQ ID NO: 84.

In some embodiments herein, a recombinant chymotrypsin is a Goat (Capra hircus) chymotrypsin and comprises at least 70% (e.g., 70%, 75%, 80%, 85%, 90%, 95%, 100%, or ranges therebetween) sequence identity to SEQ ID NO: 14 and/or is encoded by a nucleic acid having at least 70% (e.g., 70%, 75%, 80%, 85%, 90%, 95%, 100%, or ranges therebetween) sequence identity to SEQ ID NO: 86.

In some embodiments herein, a recombinant chymotrypsin is a Rat (Rattus norvegicus) chymotrypsin and comprises at least 70% (e.g., 70%, 75%, 80%, 85%, 90%, 95%, 100%, or ranges therebetween) sequence identity to SEQ ID NO: 15 and/or is encoded by a nucleic acid having at least 70% (e.g., 70%, 75%, 80%, 85%, 90%, 95%, 100%, or ranges therebetween) sequence identity to SEQ ID NO: 87.

In some embodiments herein, a recombinant chymotrypsin is a Rabbit (Oryctolagus cuniculus) chymotrypsin and comprises at least 70% (e.g., 70%, 75%, 80%, 85%, 90%, 95%, 100%, or ranges therebetween) sequence identity to SEQ ID NO: 16 and/or is encoded by a nucleic acid having at least 70% (e.g., 70%, 75%, 80%, 85%, 90%, 95%, 100%, or ranges therebetween) sequence identity to SEQ ID NO: 88.

In some embodiments herein, a recombinant chymotrypsin is a Whale (Balaenoptera acutorostrata scammoni) chymotrypsin and comprises at least 70% (e.g., 70%, 75%, 80%, 85%, 90%, 95%, 100%, or ranges therebetween) sequence identity to SEQ ID NO: 17 and/or is encoded by a nucleic acid having at least 70% (e.g., 70%, 75%, 80%, 85%, 90%, 95%, 100%, or ranges therebetween) sequence identity to SEQ ID NO: 89.

In some embodiments herein, a recombinant chymotrypsin is a Chicken (Gallus gallus) chymotrypsin and comprises at least 70% (e.g., 70%, 75%, 80%, 85%, 90%, 95%, 100%, or ranges therebetween) sequence identity to SEQ ID NO: 18 and/or is encoded by a nucleic acid having at least 70% (e.g., 70%, 75%, 80%, 85%, 90%, 95%, 100%, or ranges therebetween) sequence identity to SEQ ID NO: 90.

In some embodiments herein, a recombinant chymotrypsin is a Crocodile (Crocodylus porosus) chymotrypsin and comprises at least 70% (e.g., 70%, 75%, 80%, 85%, 90%, 95%, 100%, or ranges therebetween) sequence identity to SEQ ID NO: 19 and/or is encoded by a nucleic acid having at least 70% (e.g., 70%, 75%, 80%, 85%, 90%, 95%, 100%, or ranges therebetween) sequence identity to SEQ ID NO: 91.

In some embodiments herein, a recombinant chymotrypsin is a Bumble bee (Bombus vosnesenskii) chymotrypsin and comprises at least 70% (e.g., 70%, 75%, 80%, 85%, 90%, 95%, 100%, or ranges therebetween) sequence identity to SEQ ID NO: 20 and/or is encoded by a nucleic acid having at least 70% (e.g., 70%, 75%, 80%, 85%, 90%, 95%, 100%, or ranges therebetween) sequence identity to SEQ ID NO: 92.

In some embodiments herein, a recombinant chymotrypsin is a Asian honeybee (Apis cerena cerena) chymotrypsin and comprises at least 70% (e.g., 70%, 75%, 80%, 85%, 90%, 95%, 100%, or ranges therebetween) sequence identity to SEQ ID NO: 21 and/or is encoded by a nucleic acid having at least 70% (e.g., 70%, 75%, 80%, 85%, 90%, 95%, 100%, or ranges therebetween) sequence identity to SEQ ID NO: 93.

In some embodiments herein, a recombinant chymotrypsin is an Ant (Solenopsis invicta) chymotrypsin and comprises at least 70% (e.g., 70%, 75%, 80%, 85%, 90%, 95%, 100%, or ranges therebetween) sequence identity to SEQ ID NO: 22 and/or is encoded by a nucleic acid having at least 70% (e.g., 70%, 75%, 80%, 85%, 90%, 95%, 100%, or ranges therebetween) sequence identity to SEQ ID NO: 94.

In some embodiments herein, a recombinant chymotrypsin is a Butterfly (Danaus plexippus Plexippus) chymotrypsin and comprises at least 70% (e.g., 70%, 75%, 80%, 85%, 90%, 95%, 100%, or ranges therebetween) sequence identity to SEQ ID NO: 23 and/or is encoded by a nucleic acid having at least 70% (e.g., 70%, 75%, 80%, 85%, 90%, 95%, 100%, or ranges therebetween) sequence identity to SEQ ID NO: 95.

In some embodiments herein, a recombinant chymotrypsin is a Firefly (Photinus pyralis) chymotrypsin and comprises at least 70% (e.g., 70%, 75%, 80%, 85%, 90%, 95%, 100%, or ranges therebetween) sequence identity to SEQ ID NO: 24 and/or is encoded by a nucleic acid having at least 70% (e.g., 70%, 75%, 80%, 85%, 90%, 95%, 100%, or ranges therebetween) sequence identity to SEQ ID NO: 96.

In some embodiments herein, a recombinant chymotrypsin is a Mosquito (Aedes aegypti) chymotrypsin and comprises at least 70% (e.g., 70%, 75%, 80%, 85%, 90%, 95%, 100%, or ranges therebetween) sequence identity to SEQ ID NO: 25 and/or is encoded by a nucleic acid having at least 70% (e.g., 70%, 75%, 80%, 85%, 90%, 95%, 100%, or ranges therebetween) sequence identity to SEQ ID NO: 97.

In some embodiments herein, a recombinant chymotrypsin is a Beetle (Tenebrio molitor) chymotrypsin A0A8J6LPH0 and comprises at least 70% (e.g., 70%, 75%, 80%, 85%, 90%, 95%, 100%, or ranges therebetween) sequence identity to SEQ ID NO: 26 and/or is encoded by a nucleic acid having at least 70% (e.g., 70%, 75%, 80%, 85%, 90%, 95%, 100%, or ranges therebetween) sequence identity to SEQ ID NO: 98.

In some embodiments herein, a recombinant chymotrypsin is a Beetle (Tenebrio molitor) chymotrypsin A0A8J6HC31 and comprises at least 70% (e.g., 70%, 75%, 80%, 85%, 90%, 95%, 100%, or ranges therebetween) sequence identity to SEQ ID NO: 27 and/or is encoded by a nucleic acid having at least 70% (e.g., 70%, 75%, 80%, 85%, 90%, 95%, 100%, or ranges therebetween) sequence identity to SEQ ID NO: 99.

In some embodiments herein, a recombinant chymotrypsin is a Beetle (Tenebrio molitor) chymotrypsin A0A8J6GYN2 and comprises at least 70% (e.g., 70%, 75%, 80%, 85%, 90%, 95%, 100%, or ranges therebetween) sequence identity to SEQ ID NO: 28 and/or is encoded by a nucleic acid having at least 70% (e.g., 70%, 75%, 80%, 85%, 90%, 95%, 100%, or ranges therebetween) sequence identity to SEQ ID NO: 100.

In some embodiments herein, a recombinant chymotrypsin is a Moth (Diatraea saccharalis) chymotrypsin A0A9N9R2F0 and comprises at least 70% (e.g., 70%, 75%, 80%, 85%, 90%, 95%, 100%, or ranges therebetween) sequence identity to SEQ ID NO: 29 and/or is encoded by a nucleic acid having at least 70% (e.g., 70%, 75%, 80%, 85%, 90%, 95%, 100%, or ranges therebetween) sequence identity to SEQ ID NO: 101.

Certain embodiments of the present disclosure include compositions comprising a polynucleotide encoding a recombinant chymotrypsin polypeptide and having at least 70% (e.g., 70) %, 75%, 80%, 85%, 90%, 95%, 100%, or ranges therebetween) sequence identity to one of SEQ ID NOS: 73-144 and 291-292. In some embodiments, the polynucleotide further comprises a promoter sequence and/or encoding one or more of a secretion signal, a pro-peptide, or a purification tag.

Embodiments of the present disclosure include compositions comprising a chymotrypsin polypeptide comprising at least 70% (e.g., 70%, 75%, 80%, 85%, 90%, 95%, 100%, or ranges therebetween) sequence identity to one of SEQ ID NOS: 1-72 or SEQ ID NOS: 289-290.

In some embodiments, the recombinant chymotrypsin is capable of decreased autoproteolysis and decreased non-specific autoproteolysis as compared to native bovine chymotrypsin. In some embodiments, the chymotrypsin is capable of increased thermostability and activity as compared to a wild-type chymotrypsin. In some embodiments, the chymotrypsin is capable of increased cleavage specificity at mildly acidic digestion conditions as compared to a native bovine chymotrypsin. In some embodiments, the chymotrypsin is capable of cleaving at a tyrosine and a phenylalanine residue and significantly reduced cleavages at a tryptophan residue. In some embodiments, the chymotrypsin is capable of cleaving at a leucine and an alanine residue. In some embodiments, the chymotrypsin is capable of cleaving at a tyrosine residue.

In some embodiments, the chymotrypsin polypeptide further comprises a yeast secretion signal. In some embodiments, the chymotrypsin polypeptide further comprises a purification tag.

Embodiments of the present disclosure also include methods of producing a recombinant chymotrypsin comprising growing a cell under conditions in which the recombinant chymotrypsin polypeptide is expressed. In some embodiments, the method further comprises purifying the recombinant chymotrypsin polypeptide.

Embodiments of the present disclosure also include methods comprising a method of producing a recombinant chymotrypsin comprising screening enzymes from a source for chymotrypsins or chymotryptic-like proteases and engineering a gene construct encoding a polypeptide having at least 70% (e.g., 70%, 75%, 80%, 85%, 90%, 95%, 100%, or ranges therebetween) sequence identity to one of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70, SEQ ID NO: 71, SEQ ID NO: 72; SEQ ID NO 289; or SEQ ID NO 290.

Embodiments of the present disclosure also include compositions comprising a polynucleotide encoding a chymotrypsin polypeptide comprising at least 70% (e.g., 70%, 75%, 80%, 85%, 90%, 95%, 100%, or ranges therebetween) sequence identity to one of SEQ ID NOS: 1-72 or SEQ ID NOS 289-290.

In some embodiments, a polynucleotide herein has at least 70% (e.g., 70%, 75%, 80%, 85%, 90%, 95%, 100%, or ranges therebetween) sequence identity to one of SEQ ID NOS: 71-144 or SEQ ID NOS 291-292.

Embodiments of the present disclosure also include methods comprising a method of producing a recombinant chymotrypsin comprising: (a) integrating a polynucleotide encoding a chymotrypsin or chymotrypsin-like polypeptide into host cell; and (b) cultivating cell in media to induce expression of the recombinant chymotrypsin.

In some embodiments, the chymotrypsin or chymotrypsin-like polypeptide has at least 70% (e.g., 70%, 75%, 80%, 85%, 90%, 95%, 100%, or ranges therebetween) sequence identity to one or SEQ ID NOS 1-72 or SEQ ID NOS 289-290.

In some embodiments, the polynucleotide has at least 70% (e.g., 70%, 75%, 80%, 85%, 90%, 95%, 100%, or ranges therebetween) sequence identity to one or SEQ ID NOS 71-144 or SEQ ID NOS 291-292.

In some embodiments herein, a host cell is a yeast cell. In some embodiments, the yeast cell is a Pichia pastoris cell. In some embodiments, a polynucleotide described herein is stably incorporated into the host cell.

In some embodiments, methods comprise harvesting expressed recombinant chymotrypsin. In some embodiments, the method comprise purifying the expressed recombinant chymotrypsin. In some embodiments, purifying comprises ion exchange chromatography and/or IMAC chromatography.

In some embodiments, a recombinant chymotrypsin herein comprises a secretion signal and/or a purification tag.

Embodiments of the present disclosure also include methods comprising a method comprising incubating a recombinant chymotrypsin zymogen expressed in Pichia pastoris in a culture supernatant to allow for the recombinant chymotrypsin zymogen self-maturation and self-activation.

Embodiments of the present disclosure also include methods comprising a method comprising incubating a recombinant chymotrypsin zymogen expressed in Pichia pastoris in a culture supernatant and contacting the recombinant chymotrypsin with an exoprotease to induce the recombinant chymotrypsin complete maturation and activation. In some embodiments, the exoprotease is aminopeptidase.

Embodiments of the present disclosure also include methods comprising a method comprising the recombinant chymotrypsin zymogen maturation and activation with pre-activated recombinant chymotrypsin, native chymotrypsin extracted from an animal, trypsin or other protease or combination of protease and exonuclease.

Embodiments of the present disclosure also include methods comprising a method comprising characterizing the cellular activities, interactions, functions, compositions, and structures of a biotherapeutic protein using a recombinant chymotrypsin.

Embodiments of the present disclosure also include methods comprising a method comprising identifying and monitoring one or more protein biomarkers in a sample using a recombinant chymotrypsin.

In some embodiments, provided herein are methods comprising characterization of biotherapeutic and other proteins with peptide mapping and proteomic approaches using a recombinant chymotrypsin.

Embodiments of the present disclosure also include methods comprising a method comprising applying a recombinant chymotrypsin for clinical use. In some embodiments, the clinical uses comprise treatment of wounds, inflammation, and other disorders.

Embodiments of the present disclosure also include methods comprising a method comprising the recombinant chymotrypsin zymogen maturation and activation with the proteases secreted into the culture media by Pichia pastoris.

Embodiments of the present disclosure also include methods comprising a method comprising incubating a recombinant chymotrypsin zymogen expressed in Pichia pastoris in a culture supernatant to allow for the recombinant chymotrypsin zymogen self-maturation and followed by aminopeptidase treatment to induce activation.

### BRIEF DESCRIPTION OF THE DRAWINGS

FIG. 1 shows the batch-to-batch variability in the enzymatic activity of batches of Promega's MS grade chymotrypsin.

FIGS. 2A-2B show digestion of panitumumab with Promega's MS-grade chymotrypsin. FIG. 2A shows digested peptides which were separated by RP-UPLC-UV and analyzed with MS/MS. FIG. 2B shows the total ion intensity for peptides produced with specific C-terminal amino acids. The digests show that chymotrypsin cleaves after F/Y/W and other short aliphatic residues. These cleavage patterns increase the unreliability and complexity of the analysis of biotherapeutic proteins and hinders the use of the enzyme for this application.

FIG. 3 shows prominent autoproteolysis of Promega's MS-grade chymotrypsin. Chymotrypsin was incubated overnight in reaction buffer and the autoproteolytic peptides were separated by RP-UPLC-UV.

FIG. 4 shows a phylogram of estimated phylogeny of chymotrypsins and chymotrypsin-like proteases that were used in the study.

FIG. 5 shows an exemplary construct of chymotrypsin used for chromosomal integration in Pichia pastoris.

FIGS. 6A-6B show activation of recombinant chymotrypsins (rChymotrypsins) using the trypsin-facilitated method. The recombinant bovine chymotrypsin (rBovine chymotrypsin) and the recombinant porcine chymotrypsin (rPorcine chymotrypsin) were expressed in the form of inactive zymogens that then were incubated with trypsin for 30 min to activate the recombinant zymogens. BSA was used as a model digestion substrate to test the recombinant chymotrypsin activation. BSA was digested if a recombinant chymotrypsin was active and remained undigested if a recombinant chymotrypsin was inactive. FIG. 6A shows the recombinant chymotrypsins before activation. BSA remained undigested indicating lack of recombinant chymotrypsin activity. FIG. 6B shows BSA degraded with the activated recombinant chymotrypsins. The extent of BSA degradation correlated with recombinant chymotrypsin activity.

FIG. 7 shows human protein extract that was incubated with recombinant honeybee chymotrypsin (rHoneybee chymotrypsin) for 4 hours and analyzed with SDS-PAGE.

FIGS. 8A-8B show that pre-incubation of recombinant Bovine (rBovine) chymotrypsin with aminopeptidase enhanced rBovine chymotrypsin activity to the levels of activity of catalog chymotrypsin. The rBovine chymotrypsin was expressed in the form of a zymogen and self-activated during expression into an intermediate form of low activity. FIG. 8A shows schematics of rBovine chymotrypsin self-maturation followed by activation with aminopeptidase. FIG. 8B shows analysis of proteolytic cleavage activity.

FIGS. 9A-9B show analysis of Panitumumab antibody digested with recombinant Honeybee chymotrypsin. FIG. 9A shows RP-UPLC-UV separation and MS/MS analysis of digested peptides. FIG. 9B shows the total ion intensity for peptides with specific C-terminal amino acids. This digest reveals the enhanced specificity of recombinant honeybee chymotrypsin, primarily cleaving after F/Y residues, while cleavages after small other amino acids (H/M/V/T/K) are notably suppressed. Leucine cleavages are still present. This specificity profile contributes to more reliable and straightforward analyses of biotherapeutic proteins, showcasing the enzyme's suitability for such applications.

FIG. 10 shows RP-UPLC-UV analysis of Myoglobin digested with recombinant honeybee chymotrypsin under various reaction conditions. The chromatogram shows RP-UPLC-UV separation of digested peptides. Mildly acidic pH provides the best conditions for enzyme performance. Non-specific cleavages are further minimized at these conditions. Mildly acidic conditions are also ideal for accurate non-enzymatic PTM characterization.

FIGS. 11A-11B show snake chymotrypsins amino acid sequences. FIG. 11A shows sequence alignment of V. lebetina VLCTLP and Daboia siamensis E5L0E4 and its T62R/N81S/N83S/N134Q mutant. FIG. 11B shows sequence identity of the snake chymotrypsins.

FIG. 12 shows RP-UPLC-UV analysis of insulin-B chain digested with the recombinant snake chymotrypsin E5L0E4 variant T62R/N81S/N83S/N134Q. The digest indicates that the chymotrypsin has a strict tyrosine-specific cleavage activity.

FIG. 13 demonstrates recombinant honeybee chymotrypsin resistance to autoproteolysis. Recombinant honeybee chymotrypsin was incubated overnight in the reaction buffer and subsequent RP-UPLC-UV analysis revealed minimal generation of autoproteolytic peptides, indicating resistance of the enzyme to autoproteolysis.

FIGS. 14A-14B show RP-UPLC-UV analysis of insulin-B chain digested with wildtype recombinant honeybee chymotrypsin or recombinant honeybee chymotrypsin variant K29G/Q62V/R84P/V191C. The preliminary results show that the variant chymotrypsin has higher catalytic activity. Insulin-B chain peptide fragments due to tyrosine cleavages are FI-Y16 and L17-A30; and the peptides due to phenylalanine cleavages are F25-A30 and L17-F24. The prominent intact insulin-B chain peak in the FIG. 14A means that the mutant chymotrypsin is more active than the wildtype.

FIGS. 15A-15B show RP-UPLC-UV analysis of Panitumumab antibody digested with wildtype recombinant honeybee chymotrypsin or recombinant honeybee chymotrypsin variant K29G/Q62V/R84P/V191C under harsh conditions. FIG. 15A shows digestion with wildtype honeybee chymotrypsin. FIG. 15B shows digest with the recombinant honeybee chymotrypsin variant K29G/Q62V/R84P/V191C. FIG. 15A shows that wildtype recombinant honeybee chymotrypsin had a reduced proteolytic activity compared to the recombinant honeybee chymotrypsin variant K29G/Q62V/R84P/V191C, which showed higher proteolytic activity at harsh conditions in FIG. 15B. The higher proteolytic activity is demonstrated by the higher number of peptide peaks in FIG. 15B.

FIG. 16 shows RP-UPLC-UV analysis of insulin-B chain digested with recombinant honeybee chymotrypsin variant G179S/A18OS/V191C/P204ST/D211V. Insulin-B chain peptide fragments due to tyrosine cleavages are F1-Y16 and L17-A30; the peptides due to phenylalanine cleavages are F25-A30 and L17-F2; the peptides due to leucine cleavages are F1-L15 and Y16-A30, and the peptides due to alanine cleavages are F1-A14 and L15-A30. At reaction conditions with a pH greater than or equal to 9, the variant is inhibited, and the insulin-B chain remains intact. However, at reaction conditions with a pH less than or equal to 7.5, the variant cleaves primarily after leucine and alanine. Recombinant honeybee chymotrypsin variant A180S/V191C/P204DI/D211G showed similar results.

FIG. 17 shows RP-UPLC-UV analysis of insulin-B chain digested with recombinant honeybee chymotrypsin variant D211G. Insulin-B chain peptide fragments due to leucine cleavages are F1-L15 and Y16-A30, the peptides due to alanine cleavages are F1-A14 and L15-A30, the peptides due to phenylalanine cleavages F25-A30, and fragments due to tyrosine cleavages are F1-Y16 and L17-A30. At reaction pH of 7.5 or lower, the mutant cleaves primarily after leucine and alanine, at reaction pH of 9 and above the mutant cleaves after tyrosine, phenylalanine, and leucine.

FIG. 18 shows RP-UPLC-UV analysis of Insulin-B chain digested with recombinant honeybee chymotrypsin variant V199G. Insulin-B chain peptide fragments due to tyrosine cleavages are F1-Y16 and L17-A30; the peptides due to phenylalanine cleavages are F25-A30 and L17-F24; the peptides due to leucine cleavages are F1-L15 and Y16-A30), and the peptides due to alanine cleavages are F1-A14 and L15-A30. At reaction conditions with pH greater than or equal to 6, recombinant honeybee chymotrypsin variant V199G cleaves primarily after Y/F. However, at reaction conditions with pH 4, the variant cleaves primarily after leucine and alanine. Similarly, the recombinant honeybee chymotrypsin variants E153I, F173L, F229N/1231-245, and C127A/1128C/V190C showed induced leucine and alanine cleavages at pH 4.

### DETAILED DESCRIPTION

Provided herein are compositions, methods, and kits related to chymotrypsin production for protein analytical applications such as peptide mapping and proteomics and clinical use including treatment of wounds, ulcers, asthma, and other applications.

### 1. DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a,” “an,” and “the” include plural reference unless the context clearly dictates otherwise. Thus, for example, reference to “a domain” is a reference to one or more domains and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, a “gene construct” broadly refers to any gene of interest associated with a regulatory sequence which is inserted in the correct orientation into an expression vector to make a protein in vivo or in vitro. In some embodiments, the gene construct is used to control gene expression. In some embodiments, the gene construct comprises a promoter region, a gene coding region, secretion sequence, pro-peptide and a terminator/stop region. In some embodiments, the gene construct is an artificially-designed segment of DNA borne on an expression vector that can be used to incorporate genetic material into a host.

As used herein, the term “source” refers to the organism from which a biological molecule (e.g., nucleic acid, protein, nucleic acid sequence, protein sequence, etc.) was obtained or derived. Exemplary sources include organisms of the bacteria kingdoms (e.g., genus Streptomyces, genus Pseudoalteromonas, Thermobifida fusca, class Gammaproteobacteria), the fungi kingdom (e.g., yeast, Beauveria bassiana, Metarhizium anisopliae, Scytalidium lignicola), the plant kingdom, and the animal kingdom (e.g., insects (e.g., honeybees, bumble bees, Asian honeybees, ants, mosquitos, beetles, moths, butterflies, fireflies), mammals (e.g., whales, horses, sheep, goats, pigs, rodents (e.g., rats, mice), rabbits, cattle (e.g., cows), primates (e.g., monkeys), birds, (e.g., poultry (e.g., chickens, ducks), reptiles (e.g., snakes, crocodiles), amphibians, fish (e.g., cod, zebrafish), crustaceans, and/or worms). In some embodiments, a source may be at any stage of development. In some embodiments, a source may be a transgenic animal, genetically-engineered animal, and/or a clone. The “source” may be where a sequence of a polypeptide or polynucleotide was derived, and need not be where the physical polypeptide or polynucleotide was obtained.

As used herein, the term “host” refers to biological system (e.g., “host system”) in which a biological molecule is placed, expressed, produced, etc. that is not the source of the biological molecule (e.g., the biological molecule is not native to the host). Examples of host systems include a cell, cell lysate, non-cellular expression system, organism, etc.

As used herein, the term “expression vector” broadly refers to a polynucleotide (e.g., plasmid) or virus (e.g., AAV) for the introduction of a gene of interest (e.g., encoding a chymotrypsin) into a host system.

As used herein, the term “subject” broadly refers to any animal, including human and non-human animals (e.g., dogs, cats, cows, horses, sheep, poultry, fish, crustaceans, etc.). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the phrase “symptoms are reduced” means when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom.

As used herein, the term “patient” typically refers to a subject that is being treated for a disease or condition.

As used herein, the term “preventing” refers to prophylactic steps taken to reduce the likelihood of a subject (e.g., an at-risk subject) from developing or suffering from a particular disease, disorder, or condition (e.g., asthma). The likelihood of the disease, disorder, or condition occurring in the subject need not be reduced to zero for the preventing to occur; rather, 10) if the steps reduce the risk of a disease, disorder, or condition across a population, then the steps prevent the disease, disorder, or condition for an individual subject within the scope and meaning herein.

As used herein, the term “treatment” (also “treat” or “treating”) refer to obtaining a desired pharmacologic and/or physiologic effect against a particular disease, disorder, or condition. Preferably, the effect is therapeutic, i.e., the effect partially or completely alleviates, ameliorates, relives, inhibits, delays onset of, reduces severity of, and/or reduces frequency, incidence, or severity of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder, and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

As used herein, the terms “administration” and “administering” refer to the act of introducing a substance, such as a drug, prodrug, or other agent, or therapeutic treatment to a subject or in vivo, in vitro, or ex vivo cells, tissues, and organs. In general, any route of administration may be utilized including, for example, parenteral (e.g., intravenous), oral, topical, subcutaneous, peritoneal, intra-arterial, inhalation, vaginal, rectal, nasal, introduction into the cerebrospinal fluid, or instillation into body compartments. Exemplary routes of administration to the human body can be by parenteral administration (e.g., intravenously, subcutaneously, etc.), orally, etc.

As used herein, the term “approximately” and “about” is intended to encompass normal statistical variation as would be understood by those of ordinary skill in the art as appropriate to the relevant context. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “human” means a human is an embryo, a fetus, an infant, a child, a teenager, an adult, or a senior citizen.

As used herein, the terms “co-administration” and “co-administering” refer to the administration of at least two agent(s) or therapies to a subject. In some embodiments, the co-administration of two or more agents or therapies is concurrent (e.g., in a single formulation/composition or in separate formulations/compositions). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “mutation” refers to a change or difference in an amino acid or nucleic acid sequence relative to a reference sequence (e.g., a wild-type sequence). It is intended that the term encompass substitutions, insertions, and deletions. A mutation may be naturally occurring (e.g., a “natural mutation”), resulting from, for example, an error in DNA replication during cell division, exposure to mutagens, a viral infection, etc., or intentionally introduced (e.g., an “artificial mutation”) into a sequence.

As used herein, the term “variant” refers to a recombinant enzyme containing mutation(s) as opposite to a “wild-type” or “native” recombinant enzyme that contains natural, non-mutated amino acid sequence.

As used herein, the term “chymotrypsin” refers to a digestive enzyme belonging to the serine protease super family of enzymes. It uses an active serine residue to perform hydrolysis on the C-terminus of the amino acids of other proteins with the cleavage preference toward aromatic residues. This protease family can also cleave at other residues. The term “chymotrypsin-like” refers to other enzymes (e.g., non-digestive) having similar catalytic activities to chymotrypsin (i.e., preferential hydrolysis on the C-terminus of the aromatic amino acids of other proteins). The term “recombinant chymotrypsin” means a chymotrypsin obtained from a source and expressed in a heterologous host. As used herein, a chymotrypsin or a chymotrypsin-like protease can be expressed, and, accordingly, claimed, as recombinant chymotrypsin.

The term “amino acid” refers to natural amino acids, unnatural amino acids, and amino acid analogs, all in their D and L stereoisomers, unless otherwise indicated, if their structures allow such stereoisomeric forms. Embodiments herein refer to various amino acid abbreviations (single-letter or three-letter abbreviations) that will be understood by those in the field. The term “proteinogenic amino acids” refers to the 20 amino acids coded for in the human genetic code, and includes alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V). Selenocysteine and pyroolysine may also be considered proteinogenic amino acids.

The term “non-proteinogenic amino acid” refers to an amino acid that is not naturally-encoded or found in the genetic code of any organism, and is not incorporated biosynthetically into proteins during translation. Non-proteinogenic amino acids may be “unnatural amino acids” (amino acids that do not occur in nature) or “naturally-occurring non-proteinogenic amino acids” (e.g., norvaline, ornithine, homocysteine, etc.). Examples of non-proteinogenic amino acids include, but are not limited to, azetidinecarboxylic acid, 2-aminoadipic acid, 3-aminoadipic acid, beta-alanine, naphthylalanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-alkylglycine including N-methylglycine, N-methylisoleucine, N-alkylpentylglycine including N-methylpentylglycine. N-methylvaline, naphthylalanine, norvaline, norleucine (“Norleu”), octylglycine, ornithine, pentylglycine, pipecolic acid, thioproline, homolysine, and homoarginine. Non-proteinogenic also include D-amino acid forms of any of the amino acids herein, as well as non-alpha amino acid forms of any of the amino acids herein (beta-amino acids, gamma-amino acids, delta-amino acids, etc.), all of which are in the scope herein and may be included in peptides herein.

The term “amino acid analog” refers to an amino acid (e.g., natural or unnatural, proteinogenic or non-proteinogenic) where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide, and S-(carboxymethyl)-cysteine sulfone.

The term “peptide” refers to an amino acid polymer of about 50 amino acids or less in length. A peptide may comprise natural amino acids, non-natural amino acids, proteinogenic amino acids, non-proteinogenic amino acids, amino acid analogs, and/or modified amino acids. A peptide may be a subsequence of naturally occurring protein or a non-natural (artificial) sequence. A peptide may be a partial oligomer from a protein (e.g., a product cleavage of a protein).

The term “artificial” refers to compositions and systems that are designed or prepared synthetically, and are not naturally occurring. For example, an artificial peptide, peptoid, or nucleic acid is one comprising a non-natural sequence (e.g., a peptide without 100% identity with a naturally-occurring protein or a fragment thereof).

The term “conservative,” in reference to an amino acid substitution, refers to the substitution of an amino acid in a peptide or polypeptide with another amino acid having similar chemical properties, such as size or charge. For purposes of the present disclosure, each of the following eight groups contains amino acids that are conservative substitutions for one another: 1) Alanine (A) and Glycine (G); 2) Aspartic acid (D) and Glutamic acid I; 3) Asparagine (N) and Glutamine (Q); 4) Arginine I and Lysine (K); 5) Isoleucine (I), Leucine (L), Methionine (M), and Valine (V); 6) Phenylalanine (F), Tyrosine (Y), and Tryptophan (W); 7) Serine(S) and Threonine (T); and 8) Cysteine I and Methionine (M).

Naturally occurring residues may be divided into classes based on common side chain properties, for example: polar positive (or basic) (histidine (H), lysine (K), and arginine I); polar negative (or acidic) (aspartic acid (D), glutamic acid I); polar neutral (serine(S), threonine (T), asparagine (N), glutamine (Q)); non-polar aliphatic (alanine (A), valine (V), leucine (L), 25 isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

The term “sequence identity” refers to the degree of which two polymer sequences (e.g., peptide, polypeptide, protein, nucleic acid, etc.) have the same sequential composition of monomer subunits. The term “sequence similarity” or “sequence homology” refers to the degree with which two polymer sequences (e.g., peptide, polypeptide, protein, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.); (2) determining the number of positions containing identical (or similar) monomers (e.g., same amino acids occurs in both sequences, similar amino acid occurs in both sequences) to yield the number of matched positions; (3) dividing the number of matched positions by the total number of positions in the comparison window (e.g., the length of the longer sequence, the length of the shorter sequence, a specified window); and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% (e.g., 70%, 75%, 80%, 85%, 90%, 95%, 100%, or ranges therebetween) sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any peptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO:Z (e.g., 20) amino acids) may have up to X substitutions (e.g., 2) relative to SEQ ID NO:Z, and may therefore also be expressed as “having X (e.g., 2) or fewer substitutions relative to SEQ ID NO:Z.”

The terms “exoprotease” and “exopeptidase” as used herein means a protease that hydrolyzes peptide bonds at the termini of peptides or proteins.

The term “protease” and “peptidase” as used herein are interchangeable and mean a proteolytic enzyme that breaks down protein (e.g., a exopeptidase, aminopeptidase, dipeptidyl peptidase, carboxypeptidase, serine type protease, metalloprotease, cysteine type protease, peptidyl dipeptidase, omega peptidase, endopeptidase, serine protease, cysteine protease, aspartic protease).

The terms “endopeptidase” and “endoprotease” as used herein means any peptidase that catalyzes the hydrolysis of peptide bonds in the interior of a polypeptide chain or protein.

The term “cleavage specificity” refers to the protease's ability to selectively cleave peptide bonds at particular amino acid residues. High cleavage specificity indicates a strong preference for specific residues, resulting in precise and high to full cleavage of those sites, while low cleavage specificity denotes a broader range of cleavage sites with less selectivity, precision, and performance.

### 2. COMPOSITIONS

Embodiments of the present disclosure include chymotrypsin polypeptides and compositions related to recombinant chymotrypsin production.

In some embodiments, the present disclosure provides a host (e.g., a yeast cell) comprising an exogenous nucleic acid sequence encoding a chymotrypsin polypeptide (e.g., a gene construct).

In some embodiments, the chymotrypsin polypeptide comprises any one of the chymotrypsin polypeptides listed in Table 1.

In some embodiments, the exogenous nucleic acid sequence is stably integrated into a host (e.g., into the genome of a cell). In other embodiments, the exogenous nucleic acid sequence is transiently expressed in a host, (e.g., in the form of a non-integrating plasmid with an expressing gene construct). In some embodiments, the host is a cell. In some embodiments, the host is a yeast cell. In some embodiments, the yeast cell is Pichia pastoris and Saccharomyces cerevisiae. In some embodiments, the yeast cell is Pichia pastoris. In some embodiments, the host is bacterial cell. In some embodiments, the bacterial cell is Salmonella, E. coli, mycobacteria, or lactobacilli. In some embodiments, the host is a mammalian cell or a cell of other eukaryotic cell line. In some embodiments, the host is a non-cellular expression system. In some embodiments, the non-cellular expression system is a bacterial non-cellular expression system. In some embodiments, the non-cellular expression system is a mammalian non-cellular expression system. In some embodiments, the host is an organism.

In some embodiments, a chymotrypsin polypeptide comprises any one or more of a secretion signal, a pro-peptide, or a purification tag. In some embodiments, the exogenous nucleic acid sequence comprises a promoter and/or encodes one or more of a secretion signal, a pro-peptide, or a purification tag.

In some embodiments, a chymotrypsin polypeptide comprises an amino acid sequence with at least 70% (e.g. 70%, 75%, 80%, 85%, 90%, 95%, 100%, or ranges therebetween) sequence identity to any one of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO:289; SEQ ID NO: 290.

In some embodiments, provided herein are chymotrypsins having at least 70% (e.g., 70%, 75%, 80%, 85%, 90%, 95%, 100%, or ranges therebetween) sequence identity to SEQ ID NO: 3 and having an N127Q substitution relative to the wild-type Sus scrofa chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 7 and having an S81L substitution relative to the wild-type Beauveria bassiana chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 8 and having one or more (e.g., 1, 2, 3, 4) of T62R, N81S, N83S, or N134Q substitutions relative to the wild-type Daboia siamensis chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 10 and having one or more (e.g., 1, 2, 3, 4) of N26Q, N82Q, N98Q, N135Q, or N232Q substitutions relative to the wild-type Macrovipera lebetinus chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 13 and having one or more (e.g., 1, 2, 3, 4) of N26Q, N61Q, or N83Q substitutions relative to the wild-type Macrovipera lebetinus chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 34 and having one or more (e.g., 1, 2, 3, 4) of A180S, V191C, P204DI, or D211V substitutions relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 35 and having a C127A substitution relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 36 and having one or more (e.g., 1, 2, 3, 4) of C127A, 1128C, or V190C substitutions relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 37 and having one or more (e.g., 1, 2, 3, 4) of K29G, Q62V, R84P, or V191C substitutions relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 38 and having one or more (e.g., 1, 2, 3, 4) of K29G, Q62V, R84P, or V191C substitutions relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 39 and having one or more (e.g., 1, 2, 3, 4) of N28Q, K29G, Q62V, or R84P/V191C substitutions relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 40 and having an A180P substitution relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 41 and having an A180V substitution relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 42 and having a D193GP substitution relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 43 and having a D211G substitution relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 44 and having a D211H substitution relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 45 and having an E153I substitution relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 46 and having an F173L substitution relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 47 and having an F201W substitution relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 48 and having one or more (e.g., 1, 2, 3, 4) of F229A, L230N, or A231-245 substitutions relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 49 and having one or more (e.g., 1, 2, 3, 4) of F242A or Y243R substitutions relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 50 and having a G1771 substitution relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 51 and having one or more (e.g., 1, 2, 3, 4) of G179S, A180S, V191C, P204ST, or D211V substitutions relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 52 and having a K7Y substitution relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 53 and having one or more (e.g., 1, 2, 3, 4) of K7Y, F242A, or Y243R substitutions relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 54 and having one or more (e.g., 1, 2, 3, 4) of K7Y, S228A, F229N, or A231-245 substitutions relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 55 and having one or more (e.g., 1, 2, 3, 4) of K7Y, V191C, or 4229-245 substitutions relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 56 and having one or more (e.g., 1, 2, 3, 4) of K7Y or V191C substitutions relative to the wild-type Macrovipera lebetinus chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 57 and having an N28Q substitution relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 58 and having a P204A substitution relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 59 and having one or more (e.g., 1, 2, 3, 4) of P204C or C205P substitutions relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 60 and having a V191C substitution relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 61 and having one or more (e.g., 1, 2, 3, 4) of V191C or A229-245 substitutions relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 62 and having a V199G substitution relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 63 and having a V199W substitution relative to the wild-type Apis mellifera chymotrypsin. In some embodiments, provided herein are polynucleotides encoding such chymotrypsins.

In some embodiments, provided herein are chymotrypsins having at least 70% (e.g., 70%, 75%, 80%, 85%, 90%, 95%, 100%, or ranges therebetween) sequence identity to SEQ ID NO: 64 and having a 4229-245 substitution relative to the wild-type Apis mellifera chymotrypsin. In some embodiments, provided herein are polynucleotides encoding such chymotrypsins.

In some embodiments, provided herein is a polynucleotide encoding a chymotrypsin polypeptide comprises, wherein the polynucleotide has at least 70% (e.g., 70%, 75%, 80%, 85%, 90%, 95%, 100%, or ranges therebetween) sequence identity to SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; SEQ ID NO: 102; SEQ ID NO: 103; SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 111; SEQ ID NO: 112; SEQ ID NO: 113; SEQ ID NO: 114; SEQ ID NO: 115; SEQ ID NO: 116; SEQ ID NO:117; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 291; or SEQ ID NO: 292.

In some embodiments, provided herein is a chymotrypsin polypeptide encoded by a polynucleotide that has at least 70% (e.g., 70%, 75%, 80%, 85%, 90%, 95%, 100%, or ranges therebetween) sequence identity to SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO:100; SEQ ID NO: 101; SEQ ID NO: 102; SEQ ID NO: 103; SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 111; SEQ ID NO: 112; SEQ ID NO: 113; SEQ ID NO: 114; SEQ ID NO: 115; SEQ ID NO: 116; SEQ ID NO: 117; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; or SEQ ID NO: 140; SEQ ID NO: 141; SEQ ID NO: 142; SEQ ID NO: 143; SEQ ID NO: 144; SEQ ID NO: 291; or SEQ ID NO: 292.

Embodiments of the present disclosure provide a nucleic acid sequence encoding a chymotrypsin polypeptide, wherein the nucleic acid comprises a promoter and/or wherein the chymotrypsin polypeptide comprises any one or more of a secretion signal, a pro-peptide, or a purification tag.

In some embodiments, a promoter is operably linked to a nucleic acid sequence encoding a chymotrypsin polypeptide. In some embodiments, the promoter can be a constitutive promoter, an inducible promoter, a repressible promoter, or a regulatable promoter. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. Exemplary yeast promoter sequences include pCyc (Medium) Promoter, pAdh (Strong) Promoter, pSte5 (Weak) Promoter, yeast ADHI promoter, cyc100 minimal promoter, cyc70) minimal promoter, cyc43 minimal promoter, cyc28 minimal promoter, cyc16 minimal promoter, pPGK1, pCYC Yeast Promoter, Yeast GPD (TDH3) Promoter, yeast mid-length ADHI promoter, Yeast CLB1 promoter region, G2/M cell cycle specific, etc. (See, e.g., US 2023/0105474 (incorporated by reference in its entirety) for sequences).

In some embodiments, a protein in a precursor form (e.g., a preprotein or a pro-peptide) is an enzyme pre-curser (e.g., a zymogen or a proenzyme). In some embodiments, the precursor form is an inactive protein (e.g., a protein that displays no catalytic activity, but may be transformed within an organism into an active enzyme (e.g., an enzyme that catalyzes a reaction involving the breakdown of proteins (e.g., a zymogen (e.g., a zymogen secreted by the pancreas (e.g., a trypsinogen and chymotrypsinogen)))).

In some embodiments, the precursor form operates to target a nascent peptide chain to the endoplasmic reticulum to enable its entry into the secretory pathway. In some embodiments, the precursor form is synthesized with an N-terminal signal peptide.

In some embodiments, the precursor form prevents protease activation inside a cell. In some embodiments, the precursor form prevents cell lysis.

In some embodiments, the precursor form is activated using a maturation method that includes an additional protease (e.g., the precursor form is activated by the cleavage of one or more of its peptide bonds using an additional protease). In some embodiments, the precursor form is activated by the active form itself (e.g., by self-maturation (e.g., a zymogenic cell can synthesize and store zymogens in inactive form). In some embodiments, the precursor form is self-activated (e.g., is activated without the assistance of an additional protease or is activated by the active form of itself).

In some embodiments, the chymotrypsin polypeptide or the nucleic acid sequence encoding a chymotrypsin polypeptide comprises or encodes an additional peptide sequence (e.g., the signal sequence or the secretion signal). In some embodiments, the precursor form comprises an additional peptide sequence (e.g., the signal sequence or the secretion signal) at the maturation site (e.g., the amino-terminus). In some embodiments, the additional sequence includes a stretch of hydrophobic residues, followed by a cleavage site that is recognized by a protease (e.g., a signal peptidase) in the endoplasmic reticulum (ER) lumen. In some embodiments, the additional sequence comprises at least 70% (e.g., 70%, 75%, 80%, 85%, 90%, 95%, 100%, or ranges therebetween) amino acid sequence identity to SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; SEQ ID NO: 186; SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; SEQ ID NO: 211; SEQ ID NO: 212; SEQ ID NO: 213; SEQ ID NO: 214; SEQ ID NO: 215; or SEQ ID NO: 216.

In some embodiments, a nucleic acid sequence encoding the amino acid sequence of a chymotrypsin herein comprises at least 70% (e.g., 70%, 75%, 80%, 85%, 90%, 95%, 100%, or ranges therebetween) sequence identity to SEQ ID NO: 217; SEQ ID NO: 218; SEQ ID NO: 219; SEQ ID NO: 220; SEQ ID NO: 221; SEQ ID NO: 222; SEQ ID NO: 223; SEQ ID NO: 224; SEQ ID NO: 225; SEQ ID NO: 226; SEQ ID NO: 227; SEQ ID NO: 228; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 236; SEQ ID NO: 237; SEQ ID NO: 238; SEQ ID NO: 239; SEQ ID NO: 240; SEQ ID NO: 241; SEQ ID NO: 242; SEQ ID NO: 243; SEQ ID NO: 244; SEQ ID NO: 245; SEQ ID NO: 246; SEQ ID NO: 247; SEQ ID NO: 248; SEQ ID NO: 249; SEQ ID NO: 250; SEQ ID NO: 251; SEQ ID NO: 252; SEQ ID NO: 253; SEQ ID NO: 254; SEQ ID NO: 255; SEQ ID NO: 256; SEQ ID NO: 257; SEQ ID NO: 258; SEQ ID NO: 259; SEQ ID NO: 260; SEQ ID NO: 261; SEQ ID NO: 262; SEQ ID NO: 263; SEQ ID NO: 264; SEQ ID NO: 265; SEQ ID NO: 267; SEQ ID NO: 268; SEQ ID NO: 269; SEQ ID NO: 270; SEQ ID NO: 271; SEQ ID NO: 272; SEQ ID NO: 273; SEQ ID NO: 275; SEQ ID NO: 276; SEQ ID NO: 277; SEQ ID NO: 278; SEQ ID NO: 279; SEQ ID NO: 280; SEQ ID NO: 281; SEQ ID NO: 282; SEQ ID NO: 283; SEQ ID NO: 284; SEQ ID NO: 285; SEQ ID NO: 286; SEQ ID NO: 287; or SEQ ID NO: 288.

In some embodiments, the additional nucleic acid sequence directs translocation into the endoplasmic reticulum (ER). In some embodiments, the additional nucleic acid sequence is an N-terminal signal sequence. In some embodiments, the additional peptide sequence is the secretion signal from S. cerevisiae pre-pro-α-factor, a 19-residue signal sequence that terminates in a signal peptidase cleavage site. In some embodiments, following the signal sequence is a 66-residue pro region, which is removed in the late Golgi by a Kex2 endoprotease. In some embodiments, downstream of the dibasic Kex2 cleavage signal is an EAEA tetrapeptide, which is trimmed by the dipeptidyl aminopeptidase Ste13. In some embodiments, the additional peptide sequence consists of two parts: a 19-amino acid N-terminal signal sequence that directs translocation into the endoplasmic reticulum (ER), followed by a 66-amino acid pro region that mediates receptor-dependent packaging into ER derived COPII transport vesicles. In some embodiments, the α-factor signal sequence is removed by a signal peptidase in the ER lumen and the pro region is cleaved by the Kex2 processing protease in the Golgi. In some embodiments, the bipartite secretion signal is effective for secreting multiple heterologous proteins in P. pastoris. In some embodiments, the α-factor secretion signal sequence is replaced with the Ost1 signal sequence, which directs co-translational translocation across the ER membrane and thereby ensures that msGFP folds only after reaching the ER lumen. In some embodiments, P. pastoris is engineered to secrete BTL2 lipase from Bacillus thermocatenulatus.

In some embodiments, the chymotrypsin polypeptide or the nucleic acid sequence encoding a chymotrypsin polypeptide comprises a purification tag (e.g., a standardized method to purify a fused recombinant protein (e.g., an affinity tag (e.g., a glutathione-S-transferase tag, a biotin tag, or a streptavidin tag, a polyhistidine tag (e.g., His-tag or His₆ (e.g., C-terminal histidine tag))))). In some embodiments the purification tag enables analysis by affinity purification (e.g., a method which makes use of specific binding interactions between molecules). In some embodiments, the purification tag is a peptide.

In some embodiments, the chymotrypsin polypeptide or the nucleic acid sequence encoding a chymotrypsin polypeptide comprises one or more (e.g., 1, 2, 3, 4, 5, 6, 7, 8, 9, 10, 11, 12, 13, 14, 15, 20, 25, 30, 40, 50, or more) substitutions relative to any one or more of the sequences listed in Table 1.

Embodiments of the present disclosure also provide nucleic acid sequence encoding a chymotrypsin polypeptide, wherein the nucleic acid sequence comprises a promoter and/or wherein the chymotrypsin polypeptide comprises any one or more of a secretion signal, a pro-peptide, or a purification tag.

Embodiments of the present disclosure also provide a chymotrypsin polypeptide comprising a chymotrypsin sequence capable of cleavage specific proteolysis (i.e., an intermolecular reaction in which the peptide chain is cleaved at a specific site) and non-specific proteolysis (i.e., an intermolecular reaction in which the peptide chain is cleaved at a non-specific site).

Embodiments of the present disclosure also provide a chymotrypsin polypeptide comprising a chymotrypsin sequence capable of increased thermostability and activity.

Embodiments of the present disclosure also provide a chymotrypsin polypeptide comprising a chymotrypsin sequence capable of cleavage specificity at any pH. In some embodiments, the chymotrypsin polypeptide comprising a chymotrypsin sequence is capable of improved cleavage specificity at low pH (e.g., pH 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, or lower, or ranges therebetween) digestion conditions. In some embodiments, the chymotrypsin polypeptide comprising a chymotrypsin sequence (e.g., a honeybee chymotrypsin) is capable of cleavage specificity at pH 8 and has improved or different cleavage specificity when compared to a different chymotrypsin (e.g., a bovine chymotrypsin). In some embodiments, the chymotrypsin polypeptide comprises a chymotrypsin sequence that is capable of cleavage specificity at high pH (e.g., pH 8.0, 8.5, 9.0, 9.5, 10.0, 10.5, 11.0 or higher, or ranges therebetween) digestion conditions. In some embodiments, a sample may be pre-treated (e.g., with NaOH or with HCl buffer, etc.) to ensure a sufficiently high or low pH to allow efficient binding of the analyte to the first component (e.g., 75% efficiency, 80% efficiency, 85% efficiency, 90% efficiency, 95% efficiency, 96% efficiency, 97% efficiency, 98% efficiency, 99% efficiency, or more, or ranges therebetween).

Embodiments of the present disclosure also provide a chymotrypsin polypeptide comprising a chymotrypsin sequence capable of cleaving at a tyrosine and a phenylalanine residue and a significantly decreased cleaving or not cleaving at a tryptophan residue.

Embodiments of the present disclosure also provide a chymotrypsin polypeptide comprising a chymotrypsin sequence capable of cleaving at a leucine and an alanine residue.

Embodiments of the present disclosure also provide a chymotrypsin polypeptide comprising a chymotrypsin sequence capable of cleaving at a tyrosine residue.

In some embodiments the chymotrypsin polypeptide further comprises a yeast secretion signal.

### 3. METHODS

Embodiments of the present disclosure also include methods for producing a chymotrypsin comprising growing a cell (e.g., a yeast cell) under conditions in which a chymotrypsin polypeptide is expressed. In some embodiments, a polynucleotide encoding chymotrypsin (or chymotrypsin-like protein) from a source is expressed in a host to produce a chymotrypsin (or chymotrypsin-like protein). In some embodiments, a mutant chymotrypsin is expressed in a host cell or system. In some embodiments, the chymotrypsin polypeptide comprises any one of the chymotrypsin polypeptides listed in Table 1. In some embodiments, the chymotrypsin polypeptide is encoded by any one of the polynucleotide sequences listed in Table 1. In some embodiments, the chymotrypsin polypeptide comprises any one or more of a secretion signal, a pro-peptide, or a purification tag. In some embodiments, the chymotrypsin polypeptide is purified or isolated

In some embodiments, the method of producing a chymotrypsin comprises screening enzymes from a source for chymotrypsins or chymotrypsin-like proteases and engineering a gene construct.

In some embodiments, the method of producing a chymotrypsin comprises one or more steps of integrating a gene construct into a host (e.g., a cell, (e.g., a yeast cell (e.g., Pichia pastoris))) to produce a transformed strain of host (e.g., a transformed cell (e.g., a transformed yeast cell (e.g., transformed Pichia pastoris cell))); cultivating the transformed strain (e.g., yeast strain (e.g., strain of Pichia pastoris (e.g., Komagataella pastoris))) in media (e.g., any growth media generally known in the field (e.g., any liquid, solid, semisolid media used to support cellular growth in an artificial environment)) to induce expression of the chymotrypsin; harvesting the expressed chymotrypsin; and purifying the expressed chymotrypsin.

In some embodiments, a chymotrypsin produced by the methods herein comprises an amino acid sequence with at least 70% (e.g., 70%, 75%, 80%, 85%, 90%, 95%, 100%, or ranges therebetween) sequence identity to any one of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 289; or SEQ ID NO: 290.

In some embodiments, a chymotrypsin produced by the methods herein is encoded by a nucleic acid sequence comprising at least 70% (e.g. 70%, 75%, 80%, 85%, 90%, 95%, 100%, or ranges therebetween) sequence identity to SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO:100; SEQ ID NO: 101; SEQ ID NO: 102; SEQ ID NO: 103; SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 111; SEQ ID NO: 112; SEQ ID NO: 113; SEQ ID NO: 114; SEQ ID NO: 115; SEQ ID NO: 116; SEQ ID NO:117; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 291; or SEQ ID NO: 292.

In some embodiments, a polynucleotide produced by the methods herein is encoded by a chymotrypsin polypeptide, wherein the polynucleotide has at least 70% (e.g., 70%, 75%, 80%, 85%, 90%, 95%, 1009%, or ranges therebetween) sequence identity to SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO:100; SEQ ID NO: 101; SEQ ID NO: 102; SEQ ID NO: 103; SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 111; SEQ ID NO: 112; SEQ ID NO: 113; SEQ ID NO: 114; SEQ ID NO: 115; SEQ ID NO: 116; SEQ ID NO:117; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 291; or SEQ ID NO: 292.

In some embodiments, a chymotrypsin polypeptide produced by the methods herein is encoded by a polynucleotide that has at least 70% (e.g., 70%, 75%, 80%, 85%, 90%, 95%, 100%, or ranges therebetween) sequence identity to SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; SEQ ID NO: 102; SEQ ID NO: 103; SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 111; SEQ ID NO: 112; SEQ ID NO: 113; SEQ ID NO: 114; SEQ ID NO: 115; SEQ ID NO: 116; SEQ ID NO: 117; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 291; or SEQ ID NO: 292.

In some embodiments, a chymotrypsin produced by the methods herein further comprises an additional peptide sequence (e.g., the signal sequence or the secretion signal). In some embodiments, the additional nucleic acid sequence comprises at least 70% (e.g., 70%, 75%, 80%, 85%, 90%, 95%, 100%, or ranges therebetween) amino acid sequence identity to SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; SEQ ID NO: 186; SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; SEQ ID NO: 211; SEQ ID NO: 212; SEQ ID NO: 213; SEQ ID NO: 214; SEQ ID NO: 215; or SEQ ID NO: 216.

In some embodiments, a chymotrypsin produced by the methods herein further comprises an additional peptide sequence (e.g., the signal sequence or the secretion signal) encoded by a polynucleotide having at least 70% (e.g., 70%, 75%, 80%, 85%, 90%, 95%, 100%, or ranges therebetween) sequence identity to SEQ ID NO: 217; SEQ ID NO: 218; SEQ ID NO: 219; SEQ ID NO: 220; SEQ ID NO: 221; SEQ ID NO: 222; SEQ ID NO: 223; SEQ ID NO: 224; SEQ ID NO: 225; SEQ ID NO: 226; SEQ ID NO: 227; SEQ ID NO: 228; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 236; SEQ ID NO: 237; SEQ ID NO: 238; SEQ ID NO: 239; SEQ ID NO: 240; SEQ ID NO: 241; SEQ ID NO: 242; SEQ ID NO: 243; SEQ ID NO: 244; SEQ ID NO: 245; SEQ ID NO: 246; SEQ ID NO: 247; SEQ ID NO: 248; SEQ ID NO: 249; SEQ ID NO: 250; SEQ ID NO: 251; SEQ ID NO: 252; SEQ ID NO: 253; SEQ ID NO: 254; SEQ ID NO: 255; SEQ ID NO: 256; SEQ ID NO: 257; SEQ ID NO: 258; SEQ ID NO: 259; SEQ ID NO: 260; SEQ ID NO: 261; SEQ ID NO: 262; SEQ ID NO: 263; SEQ ID NO: 264; SEQ ID NO: 265; SEQ ID NO: 267; SEQ ID NO: 268; SEQ ID NO: 269; SEQ ID NO: 270; SEQ ID NO: 271; SEQ ID NO: 272; SEQ ID NO: 273; SEQ ID NO: 275; SEQ ID NO: 276; SEQ ID NO: 277; SEQ ID NO: 278; SEQ ID NO: 279; SEQ ID NO: 280; SEQ ID NO: 281; SEQ ID NO: 282; SEQ ID NO: 283; SEQ ID NO: 284; SEQ ID NO: 285; SEQ ID NO: 286; SEQ ID NO: 287; or SEQ ID NO: 288.

In some embodiments, integration (i.e., integrating) is achieved by methods generally known in the field (e.g., where a genome (e.g., a gene construct) is incorporated into a host cell's DNA, and the integrated genome replicates along with the host cell's genome).

In some embodiments, cultivation (i.e., cultivating) is achieved by methods generally known in the field (e.g., laboratory methods that enable the growth of eukaryotic or prokaryotic cells in physiological conditions (e.g., selection of suitable growth conditions (e.g., the format (e.g., cell suspension or cell plated), the cell culture medium (e.g., an environment that allows for maximum cell propagation achieved through the incubator (e.g., temperature, humidity, O₂, and CO₂ tensions), and the basal cell culture medium (e.g., media containing amino acids, glucose, ions (e.g., calcium, magnesium, potassium, sodium, and phosphate), and its supplements essential for cell survival and growth).

In some embodiments, harvesting is achieved by methods generally known in the field (e.g., size exclusion (e.g., centrifugation or filtration)).

In some embodiments, purifying comprises isolating, purifying, and/or concentrating analytes (e.g., nucleic acids) from various samples. In some embodiments, purifying is achieved by a protein purification method (e.g., affinity chromatography, ion exchange chromatography, dialysis, vertical electrophoresis systems, hydrophobic chromatography, ultracentrifugation, salt, immunoaffinity, gel filtration chromatography, protein purification system, protein immobilization, centrifugation, ion exchange purification, chromatographic methods, antibodies, acrylamide, precipitation and differential solubilization, chromatography, extraction, high-performance liquid chromatography, size exclusion chromatography, polyacrylamide, blotting apparatus, Tandem Affinity Purification, (or “The TAP method”) or other methods generally known in the field).

In some embodiments, purifying is achieved by methods of protein purification generally known in the art (e.g., cation exchange purification (e.g., a protein that binds to a negatively charged ion exchange resin with an affinity for molecules having net positive surface charges), or an affinity-based protein purification method using a purification tag (e.g., a standardized method to purify a fused recombinant protein (e.g., an affinity tag (e.g., a glutathione-S-transferase tag, a biotin tag, or a streptavidin tag, a polyhistidine tag (e.g., His-tag or His₆ (e.g., C-terminal histidine tag)))))). In some embodiments the purification tag enables analysis by other types of affinity purification (e.g., a method that makes use of specific binding interactions between molecules). In some embodiments, the purification tag is a peptide. In some embodiments, purifying comprises using IMAC (Immobilized Metal Affinity Chromatography) (for example, el-nitrilotriacetic acid (Ni-NTA), nickel-iminodiacetic acid (Ni-IDA), cobalt-nitrilotriacetic acid (Co-NTA), cobalt-iminodiacetic acid (Co-IDA), copper-iminodiacetic acid (Cu-IDA), zinc-iminodiacetic acid (Zn-IDA), cobalt-based resins, nickel-based resins, nickel-based agarose resins, or other metal affinity resins).

In some embodiments, the expressed chymotrypsin is an inactive protease. In some embodiments, the inactive protease is a zymogen.

Embodiments of the present disclosure also include a method comprising incubating a chymotrypsin in a culture supernatant for a period of time (e.g., 10) minutes, 20 minutes, 30) minutes, 1 hour, 2 hours, 4 hours, 6 hours 12 hours, 18 hours, 24 hours, 36 hours, 48 hours, 3 days, 4 days, 5 days, 6 days, or more).

Embodiments of the present disclosure also include a method comprising incubating a chymotrypsin expressed in a host (e.g., Pichia) in a culture supernatant for a period of time (e.g., 10) minutes, 20) minutes, 30 minutes, 1 hour, 2 hours, 4 hours, 6 hours 12 hours, 18 hours, 24 hours, 36 hours, 48 hours, 3 days, 4 days, 5 days, 6 days, or more) and contacting the recombinant chymotrypsin with an exoprotease. In some embodiments, the exoprotease is aminopeptidase.

In some embodiments, the method of incubation (i.e., incubating) comprises incubating in the culture supernatant for a period of time a period of time sufficient to enable partial self-maturation (e.g., 10) minutes, 20) minutes, 30 minutes, 1 hour, 2 hours, 4 hours, 6 hours 12 hours, 18 hours, 24 hours, 36 hours, 48 hours, 3 days, 4 days, 5 days, 6 days, or more).

In some embodiments, the incubation for self-activation occurs during the recombinant protease expression over the course of fermentation.

Embodiments of the present disclosure also include a method comprising characterizing the cellular activities, interactions, functions, compositions, and structures of a biotherapeutic protein using a chymotrypsin by proteolysis methods generally known in the art.

In some embodiments, characterizing is by bottom-up proteomics (e.g., methods in which a protein, a protein mix, or complex protein mixture is digested by a protease (e.g., trypsin and/or chymotrypsin), then separated using a certain column (e.g., chromatography (e.g., liquid chromatography)) and then analyzed (e.g., by mass spectrometry and/or UV).

In some embodiments, characterizing is by peptide mapping (e.g., methods in which an individual protein is digested by a protease, then separated by chromatography and analyzed with mass spectrometry and/or UV.

Embodiments of the present disclosure also include methods comprising food protein sequencing, in vitro determination of food protein digestibility, and evaluating the impact(s) of food processing methods on the activity of trypsin inhibitors using a recombinant chymotrypsin described herein and/or produced by the methods described herein.

Embodiments of the present disclosure also include methods comprising clinical diagnosis of pancreatitis using a chymotrypsin described herein and/or produced by the methods described herein.

Embodiments of the present disclosure also include methods comprising treating a wound, inflammation, or other disorder of a subject using a chymotrypsin described herein and/or produced by the methods described herein.

The chymotrypsins of the disclosure may be used in any way that proteases have been used. The presence, amount, spectral distribution, emission kinetics, or specific activity of any such protein may be detected or quantified. The protein may be detected or quantified in solution, including multiphasic solutions (e.g., emulsions or suspensions) or on solid supports (e.g., particles, capillaries, or assay vessels).

In some embodiments, the chymotrypsins disclosed herein may be used to facilitate drug development by providing a comprehensive map of protein interactions associated with disease pathways.

In some embodiments, the chymotrypsins disclosed herein may be used to identify and monitor protein biomarkers (e.g., predictive, prognostic, and diagnostic biomarkers) in a sample to enable a physician to treat a subject for a disease by predicting a response to a therapy, predicting a clinical outcome, and/or indicating if a patient has a specific disease condition. In some embodiments, the disease condition is cancer, leukemia, acute myeloid leukemia. In some embodiments, the disease condition is a wound, inflammation, or other disorder of a subject.

In some embodiments, the chymotrypsins disclosed herein may be used to identify and monitor protein biomarkers associated with cancer. In some embodiments, the chymotrypsins disclosed herein may be used in oncoproteomics methods generally known in the art. In some embodiments, the chymotrypsins disclosed herein may be used to identify anticancer drugs and to personalize cancer management. In some embodiments, the chymotrypsins disclosed herein may be used to classify proteins in cancer (e.g., by microarrays, laser capture microdissection (LCM) of the tumor tissue, etc.). In some embodiments, the chymotrypsins disclosed herein may be used in tumor proteomics (e.g., LCM, MS, etc.). In some embodiments, the chymotrypsins disclosed herein may be used to identify proteins that correlate with tumor progression.

In some embodiments, the chymotrypsins disclosed herein may be used to quantify a protein of interest. In some embodiments, the recombinant chymotrypsins may be used to characterize biotherapeutic proteins and therapeutic peptides with peptide mapping.

In some embodiments, the recombinant chymotrypsins disclosed herein are prepared in a variety of formats beyond the traditional liquid and lyophilized forms. These formats include conjugation to beads, immobilization onto columns, co-immobilization with other enzymes, attachment to various carriers, various encapsulation techniques, and incorporation into different matrix types.

### 4. KITS

Embodiments of the present disclosure also include kits for proteolytically cleaving substrates (e.g., single proteins, protein mixtures, proteomes, etc.). In certain embodiments, the chymotrypsins disclosed herein may be provided as part of a kit. In some embodiments, the kit may include one or more proteases (in the form of a polypeptide, a polynucleotide, or both (e.g., a trypsin)) and a chymotrypsins disclosed herein, along with suitable reagents and instructions to enable a user to perform proteolysis methods such as those disclosed herein. The kit may also include one or more media and/or buffers such as those disclosed herein.

Buffers include citric acid or citrate buffer, MES, 1,4-Piperazinediethanesulfonic acid, or HEPE, Tris-HCl, histidine, or Bicarbonate buffer; inorganic phosphate, for example, in the form pyrophosphate or potassium phosphate; a chelator such as EDTA, CDTA or 1,2-Diaminocyclohexanetetraacetic acid; a salt such as sodium fluoride, magnesium sulfate; a surfactant or detergent such as TERGITOL® (e.g. a non-ionic nonylphenol ethoxylate), dodecyltrimethylammonium bromide (DTAB) or THESIT® (hydroxypolyethoxydodecane); a defoamer such as INDUSTROL® DF204 (organic defoamer) or MAZUR DF (silicone defoamer); a protein stabilizer such as gelatin, PRIONEX® 10% (gelatin, Type A) or albumin (e.g., BSA, HSA) or glycerol; adenosine triphosphate (ATP) or adenosine monophosphate (AMP). Other components may include polyethylene glycol, polyvinyl pyridine, crown ether, or cyclodextrin.

### 5. SEQUENCES

Certain embodiments of the present disclosure include polynucleotides and polypeptides having the sequences listed in Table 1. Embodiments herein are not limited to the sequences of Table 1. In some embodiments, a chymotrypsin or chymotrypsin-like polypeptide comprises 70% (e.g., 70%, 75%, 80%, 85%, 90%, 95%, 100%) or greater sequence identity to a polypeptide sequence of Table 1. In some embodiments, a chymotrypsin or chymotrypsin-like polypeptide is encoded by a polynucleotide having at least 70% (e.g., 70%, 75%, 80%, 85%, 90%, 95%, 100%) sequence identity to a polynucleotide sequence of Table 1. In some embodiments, provided herein are polynucleotides having at least 70% (e.g., 70%, 75%, 80%, 85%, 90%, 95%, 100%) sequence identity to a polynucleotide sequence of Table 1.

TABLE 1
Recombinant chymotrypsins and chymotrypsin-like proteases
that were investigated for expression in Pichia pastoris

| | | | Pre-pro | pre-pro |
|---|---|---|---|---|
| | | | secretion | secretion |
| | | | signal | signal |
| | Amino acid | DNA | amino acid | DNA |
| | sequence | sequence | sequence | sequence |
| Chymotrypsin name | SEQ ID NO. | SEQ ID NO. | SEQ ID NO. | SEQ ID NO. |
| Bovine (Bos taurus) chymotrypsin | SEQ ID NO: | SEQ ID NO: | SEQ ID NO: | SEQ ID NO: |
| | 1 | 73 | 145 | 217 |
| Porcine (Sus scrofa) chymotrypsin B | SEQ ID NO: | SEQ ID NO: | SEQ ID NO: | SEQ ID NO: |
| | 2 | 74 | 146 | 218 |
| Porcine (Sus scrofa) chymotrypsin | SEQ ID NO: | SEQ ID NO: | SEQ ID NO: | SEQ ID NO: |
| variant N127Q | 3 | 75 | 147 | 219 |
| Cod (Gadus morhua) chymotrypsin A | SEQ ID NO: | SEQ ID NO: | SEQ ID NO: | SEQ ID NO: |
| | 4 | 76 | 148 | 220 |
| Cod (Gadus morhua) chymotrypsin B | SEQ ID NO: | SEQ ID NO: | SEQ ID NO: | SEQ ID NO: |
| | 5 | 77 | 149 | 221 |
| Zebrafish (Danio rerio) chymotrypsin | SEQ ID NO: | SEQ ID NO: | SEQ ID NO: | SEQ ID NO: |
| | 6 | 78 | 150 | 222 |
| Beauveria bassiana chymotrypsin | SEQ ID NO: | SEQ ID NO: | SEQ ID NO: | SEQ ID NO: |
| variant S81L | 7 | 79 | 151 | 223 |
| Snake (Daboia siamensis) | SEQ ID NO: | SEQ ID NO: | SEQ ID NO: | SEQ ID NO: |
| chymotrypsin E5L0E4 variant | 8 | 80 | 152 | 224 |
| T62R/N81S/N83S/N134Q | | | | |
| Snake (Macrovipera lebetinus) | SEQ ID NO: | SEQ ID NO: | SEQ ID NO: | SEQ ID NO: |
| chymotrypsin VLCTLP | 9 | 81 | 153 | 225 |
| Snake (Macrovipera lebetinus) | SEQ ID NO: | SEQ ID NO: | SEQ ID NO: | SEQ ID NO: |
| chymotrypsin VLCTLP variant | 10 | 82 | 154 | 226 |
| N26Q/N82Q/N98Q/N135Q/N232Q | | | | |
| Snake (Macrovipera lebetinus) | SEQ ID NO: | SEQ ID NO: | SEQ ID NO: | SEQ ID NO: |
| chymotrypsin VLCTLP variant | 11 | 83 | 155 | 227 |
| N26Q/N82Q/N98Q/N135Q/N232A | | | | |
| Snake (Macrovipera lebetinus) | SEQ ID NO: | SEQ ID NO: | SEQ ID NO: | SEQ ID NO: |
| chymotrypsin VLAF | 12 | 84 | 156 | 228 |
| Snake (Macrovipera lebetinus) | SEQ ID NO: | SEQ ID NO: | SEQ ID NO: | SEQ ID NO: |
| chymotrypsin VLAF variant | 13 | 85 | 157 | 229 |
| N26Q/N61Q/N83Q | | | | |
| Goat (Capra hircus) chymotrypsin | SEQ ID NO: | SEQ ID NO: | SEQ ID NO: | SEQ ID NO: |
| | 14 | 86 | 158 | 230 |
| Rat (Rattus norvegicus) chymotrypsin | SEQ ID NO: | SEQ ID NO: | SEQ ID NO: | SEQ ID NO: |
| | 15 | 87 | 159 | 231 |
| Rabbit (Oryctolagus cuniculus) | SEQ ID NO: | SEQ ID NO: | SEQ ID NO: | SEQ ID NO: |
| chymotrypsin | 16 | 88 | 160 | 232 |
| Whale (Balaenoptera acutorostrata | SEQ ID NO: | SEQ ID NO: | SEQ ID NO: | SEQ ID NO: |
| scammoni) chymotrypsin | 17 | 89 | 161 | 233 |
| Chicken (Gallus gallus) chymotrypsin | SEQ ID NO: | SEQ ID NO: | SEQ ID NO: | SEQ ID NO: |
| | 18 | 90 | 162 | 234 |
| Crocodile (Crocodylus porosus) | SEQ ID NO: | SEQ ID NO: | SEQ ID NO: | SEQ ID NO: |
| chymotrypsin | 19 | 91 | 163 | 235 |
| Bumble bee (Bombus vosnesenskii) | SEQ ID NO: | SEQ ID NO: | SEQ ID NO: | SEQ ID NO: |
| chymotrypsin | 20 | 92 | 164 | 236 |
| Asian honeybee (Apis cerena cerena) | SEQ ID NO: | SEQ ID NO: | SEQ ID NO: | SEQ ID NO: |
| chymotrypsin | 21 | 93 | 165 | 237 |
| Ant (Solenopsis invicta) chymotrypsin | SEQ ID NO: | SEQ ID NO: | SEQ ID NO: | SEQ ID NO: |
| | 22 | 94 | 166 | 238 |
| Butterfly (Danaus plexippus Plexippus) | SEQ ID NO: | SEQ ID NO: | SEQ ID NO: | SEQ ID NO: |
| chymotrypsin | 23 | 95 | 167 | 239 |
| Firefly (Photinus pyralis) | SEQ ID NO: | SEQ ID NO: | SEQ ID NO: | SEQ ID NO: |
| chymotrypsin | 24 | 96 | 168 | 240 |
| Mosquito (Aedes aegypti) | SEQ ID NO: | SEQ ID NO: | SEQ ID NO: | SEQ ID NO: |
| chymotrypsin | 25 | 97 | 169 | 241 |
| Beetle (Tenebrio molitor) | SEQ ID NO: | SEQ ID NO: | SEQ ID NO: | SEQ ID NO: |
| chymotrypsin A0A8J6LPH0 | 26 | 98 | 170 | 242 |
| Beetle (Tenebrio molitor) | SEQ ID NO: | SEQ ID NO: | SEQ ID NO: | SEQ ID NO: |
| chymotrypsin A0A8J6HC31 | 27 | 99 | 171 | 243 |
| Beetle (Tenebrio molitor) | SEQ ID NO: | SEQ ID NO: | SEQ ID NO: | SEQ ID NO: |
| chymotrypsin A0A8J6GYN2 | 28 | 100 | 172 | 244 |
| Moth (Diatraea saccharalis) | SEQ ID NO: | SEQ ID NO: | SEQ ID NO: | SEQ ID NO: |
| chymotrypsin A0A9N9R2F0 | 29 | 101 | 173 | 245 |
| Honeybee (Apis mellifera) | SEQ ID NO: | SEQ ID NO: | SEQ ID NO: | SEQ ID NO: |
| chymotrypsin L3W4 | 30 | 102 | 174 | 246 |
| Honeybee (Apis mellifera) | SEQ ID NO: | SEQ ID NO: | SEQ ID NO: | SEQ ID NO: |
| chymotrypsin R3J2 | 31 | 103 | 175 | 247 |
| Honeybee (Apis mellifera) | SEQ ID NO: | SEQ ID NO: | SEQ ID NO: | SEQ ID NO: |
| chymotrypsin | 32 | 104 | 176 | 248 |
| Honeybee (Apis mellifera) | SEQ ID NO: | SEQ ID NO: | SEQ ID NO: | SEQ ID NO: |
| chymotrypsin | 33 | 105 | 177 | 249 |
| Honeybee (Apis mellifera) | SEQ ID NO: | SEQ ID NO: | SEQ ID NO: | SEQ ID NO: |
| chymotrypsin variant | 34 | 106 | 178 | 250 |
| A180S/V191C/P204DI/D211V | | | | |
| Honeybee (Apis mellifera) | SEQ ID NO: | SEQ ID NO: | SEQ ID NO: | SEQ ID NO: |
| chymotrypsin variant C127A | 35 | 107 | 179 | 251 |
| Honeybee (Apis mellifera) | SEQ ID NO: | SEQ ID NO: | SEQ ID NO: | SEQ ID NO: |
| chymotrypsin variant | 36 | 108 | 180 | 252 |
| C127A/I128C/V190C | | | | |
| Honeybee (Apis mellifera) | SEQ ID NO: | SEQ ID NO: | SEQ ID NO: | SEQ ID NO: |
| chymotrypsin variant | 37 | 109 | 181 | 253 |
| K29G/Q62V/R84P/V191C | | | | |
| Honeybee (Apis mellifera) | SEQ ID NO: | SEQ ID NO: | SEQ ID NO: | SEQ ID NO: |
| chymotrypsin variant | 38 | 110 | 182 | 254 |
| K29G/Q62V/R84P/V191C | | | | |
| Honeybee (Apis mellifera) | SEQ ID NO: | SEQ ID NO: | SEQ ID NO: | SEQ ID NO: |
| chymotrypsin variant | 39 | 111 | 183 | 255 |
| N28Q/K29G/Q62V/R84P/V191C | | | | |
| Honeybee (Apis mellifera) | SEQ ID NO: | SEQ ID NO: | SEQ ID NO: | SEQ ID NO: |
| chymotrypsin variant A180P | 40 | 112 | 184 | 256 |
| Honeybee (Apis mellifera) | SEQ ID NO: | SEQ ID NO: | SEQ ID NO: | SEQ ID NO: |
| chymotrypsin variant A180V | 41 | 113 | 185 | 257 |
| Honeybee (Apis mellifera) | SEQ ID NO: | SEQ ID NO: | SEQ ID NO: | SEQ ID NO: |
| chymotrypsin variant D193GP | 42 | 114 | 186 | 258 |
| Honeybee (Apis mellifera) | SEQ ID NO: | SEQ ID NO: | SEQ ID NO: | SEQ ID NO: |
| chymotrypsin variant D211G | 43 | 115 | 187 | 259 |
| Honeybee (Apis mellifera) | SEQ ID NO: | SEQ ID NO: | SEQ ID NO: | SEQ ID NO: |
| chymotrypsin variant D211H | 44 | 116 | 188 | 260 |
| Honeybee (Apis mellifera) | SEQ ID NO: | SEQ ID NO: | SEQ ID NO: | SEQ ID NO: |
| chymotrypsin variant E153I | 45 | 117 | 189 | 261 |
| Honeybee (Apis mellifera) | SEQ ID NO: | SEQ ID NO: | SEQ ID NO: | SEQ ID NO: |
| chymotrypsin variant F173L | 46 | 118 | 190 | 262 |
| Honeybee (Apis mellifera) | SEQ ID NO: | SEQ ID NO: | SEQ ID NO: | SEQ ID NO: |
| chymotrypsin variant F201W | 47 | 119 | 191 | 263 |
| Honeybee (Apis mellifera) | SEQ ID NO: | SEQ ID NO: | SEQ ID NO: | SEQ ID NO: |
| chymotrypsin variant | 48 | 120 | 192 | 264 |
| F229A/L230N/Δ231-245 | | | | |
| Honeybee (Apis mellifera) | SEQ ID NO: | SEQ ID NO: | SEQ ID NO: | SEQ ID NO: |
| chymotrypsin variant F242A/Y243R | 49 | 121 | 193 | 265 |
| Honeybee (Apis mellifera) | SEQ ID NO: | SEQ ID NO: | SEQ ID NO: | SEQ ID NO: |
| chymotrypsin variant G177I | 50 | 122 | 194 | 266 |
| Honeybee (Apis mellifera) | SEQ ID NO: | SEQ ID NO: | SEQ ID NO: | SEQ ID NO: |
| chymotrypsin variant | 51 | 123 | 195 | 267 |
| G179S/A180S/V191C/P204ST/D211V | | | | |
| Honeybee (Apis mellifera) | SEQ ID NO: | SEQ ID NO: | SEQ ID NO: | SEQ ID NO: |
| chymotrypsin variant K7Y | 52 | 124 | 196 | 268 |
| Honeybee (Apis mellifera) | SEQ ID NO: | SEQ ID NO: | SEQ ID NO: | SEQ ID NO: |
| chymotrypsin variant | 53 | 125 | 197 | 269 |
| K7Y/F242A/Y243R | | | | |
| Honeybee (Apis mellifera) | SEQ ID NO: | SEQ ID NO: | SEQ ID NO: | SEQ ID NO: |
| chymotrypsin variant | 54 | 126 | 198 | 270 |
| K7Y/S228A/F229N/Δ231-245 | | | | |
| Honeybee (Apis mellifera) | SEQ ID NO: | SEQ ID NO: | SEQ ID NO: | SEQ ID NO: |
| chymotrypsin variant K7Y/V191C | 55 | 127 | 199 | 271 |
| Honeybee (Apis mellifera) | SEQ ID NO: | SEQ ID NO: | SEQ ID NO: | SEQ ID NO: |
| chymotrypsin variant | 56 | 128 | 200 | 272 |
| K7Y/V191C/Δ229-245 | | | | |
| Honeybee (Apis mellifera) | SEQ ID NO: | SEQ ID NO: | SEQ ID NO: | SEQ ID NO: |
| chymotrypsin variant N28Q | 57 | 129 | 201 | 273 |
| Honeybee (Apis mellifera) | SEQ ID NO: | SEQ ID NO: | SEQ ID NO: | SEQ ID NO: |
| chymotrypsin variant P204A | 58 | 130 | 202 | 274 |
| Honeybee (Apis mellifera) | SEQ ID NO: | SEQ ID NO: | SEQ ID NO: | SEQ ID NO: |
| chymotrypsin variant P204C/C205P | 59 | 131 | 203 | 275 |
| Honeybee (Apis mellifera) | SEQ ID NO: | SEQ ID NO: | SEQ ID NO: | SEQ ID NO: |
| chymotrypsin variant V191C | 60 | 132 | 204 | 276 |
| Honeybee (Apis mellifera) | SEQ ID NO: | SEQ ID NO: | SEQ ID NO: | SEQ ID NO: |
| chymotrypsin variant V191C/Δ229-245 | 61 | 133 | 205 | 277 |
| Honeybee (Apis mellifera) | SEQ ID NO: | SEQ ID NO: | SEQ ID NO: | SEQ ID NO: |
| chymotrypsin variant V199G | 62 | 134 | 206 | 278 |
| Honeybee (Apis mellifera) | SEQ ID NO: | SEQ ID NO: | SEQ ID NO: | SEQ ID NO: |
| chymotrypsin variant V199W | 63 | 135 | 207 | 279 |
| Honeybee (Apis mellifera) | SEQ ID NO: | SEQ ID NO: | SEQ ID NO: | SEQ ID NO: |
| chymotrypsin variant Δ229-245 | 64 | 136 | 208 | 280 |
| Gammaproteobacteria bacterium | SEQ ID NO: | SEQ ID NO: | SEQ ID NO: | SEQ ID NO: |
| chymotrypsin-like protease | 65 | 137 | 209 | 281 |
| Pseudoalteromonas species SM1988 | SEQ ID NO: | SEQ ID NO: | SEQ ID NO: | SEQ ID NO: |
| chymotrypsin-like protease | 66 | 138 | 210 | 282 |
| Scytalidoglutamic (Scytalidium | SEQ ID NO: | SEQ ID NO: | SEQ ID NO: | SEQ ID NO: |
| lignicola) peptidase | 67 | 139 | 211 | 283 |
| Metarhizium anisopliae | SEQ ID NO: | SEQ ID NO: | SEQ ID NO: | SEQ ID NO: |
| chymotrypsinogen-like protease | 68 | 140 | 212 | 284 |
| Thermobifida fusca | SEQ ID NO: | SEQ ID NO: | SEQ ID NO: | SEQ ID NO: |
| chymotrypsinogen-like protease | 69 | 141 | 213 | 285 |
| Streptomyces griseus Protease C | SEQ ID NO: | SEQ ID NO: | SEQ ID NO: | SEQ ID NO: |
| (chymotrypsin-like protease) | 70 | 142 | 214 | 286 |
| Streptomyces species | SEQ ID NO: | SEQ ID NO: | SEQ ID NO: | SEQ ID NO: |
| chymotrypsinogen-like protease | 71 | 143 | 215 | 287 |
| Streptomyces species | SEQ ID NO: | SEQ ID NO: | SEQ ID NO: | SEQ ID NO: |
| chymotrypsinogen-like protease | 72 | 144 | 216 | 288 |
| propeptide variant | | | | |
| Daboia siamensis recombinant | SEQ ID NO: | SEQ ID NO: | NA | NA |
| chymotrypsin E5L0E4 wild-type | 289 | 291 | | |
| Beauveria bassiana chymotrypsin | SEQ ID NO: | SEQ ID NO: | NA | NA |
| wild-type | 290 | 292 | | |

### 6. EXAMPLES

### Example 1

### Shortcomings of the Commercially Available Chymotrypsin

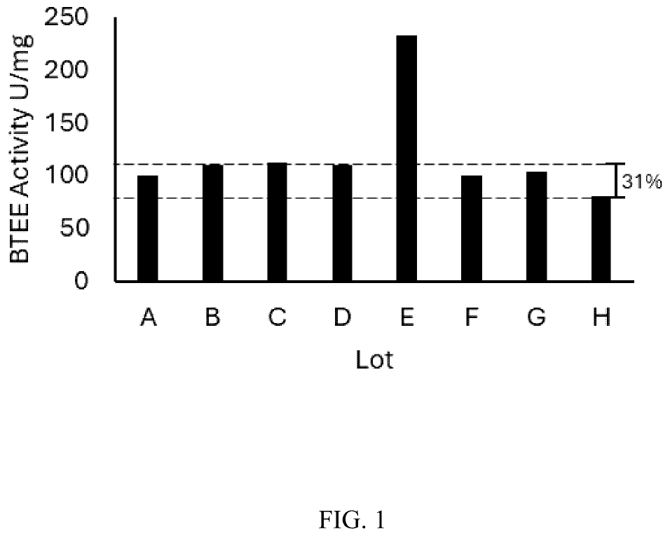

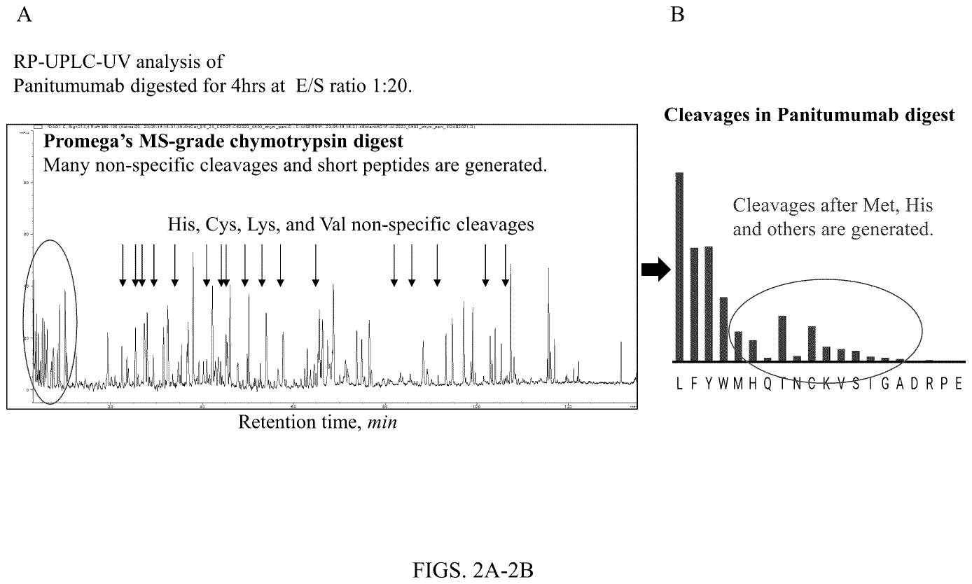

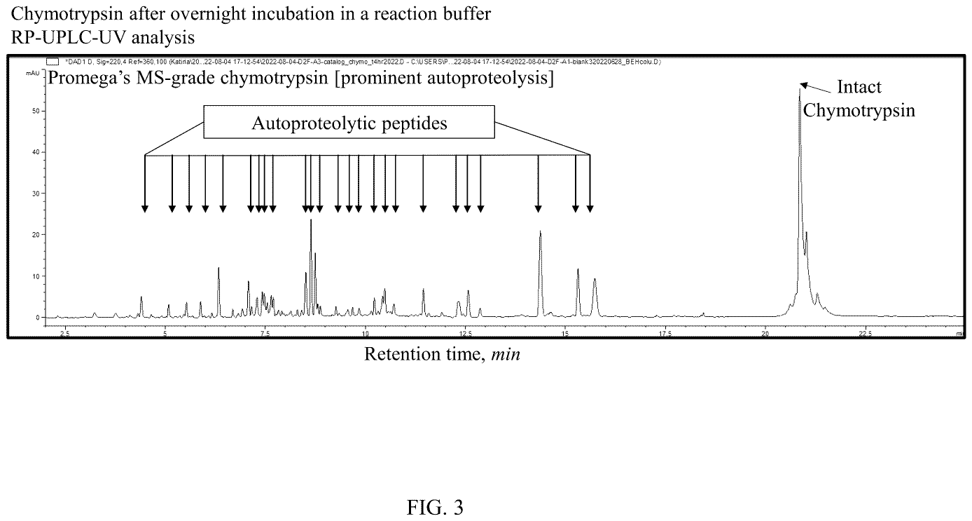

Described below are limitations of the current, commercially available chymotrypsin, using Promega's MS-grade chymotrypsin as an example. Three major chymotrypsin shortcomings are highlighted: (1) batch-to-batch variability (FIG. 1); (2) low cleavage specificity (FIG. 2); and (3) prominent autoproteolysis (FIG. 3). The batch-to-batch variability refers to the inconsistency in enzyme activity across different production lots, which leads to unpredictable results in applications. Low cleavage specificity involves the chymotrypsin tendency to cleave at non-specific, low-affinity peptide bonds, which hampers accurate protein analysis using mass spectrometry and LC-UV. Prominent autoproteolysis indicates the enzyme's tendency to degrade itself, which further complicates its use. The cumulative effect of these limitations is the reduced reliability of chymotrypsin for accurate and reproducible results in protein sequencing applications.

Batch-to-Batch Variability (FIG. 1): The figure shows the variability in activity between batches of Promega's MS grade chymotrypsins.

Poor Cleavage Specificity (FIG. 2): The figure shows high level of non-specific cleavages produced during digestion of a model antibody with Promega's MS grade chymotrypsin. In addition to the specific cleavages at aromatic residues tyrosine (Y), phenylalanine (F), and tryptophan (W), the chymotrypsin induced non-specific cleavages at leucine (L), methionine (M), histidine (H), threonine (T), cysteine (C), lysine (K), valine (V), arginine (R), and serine(S). These cleavages generate unintended peptide fragments that compromise protein analysis. For example, in peptide mapping, a peptide like ‘W.IGHIY’ (SEQ ID NO: 293) might generate non-specific peptide ‘W.IGH.’ (SEQ ID NO: 294) and ‘H.IY’ (SEQ ID NO: 295). The ‘F.GGGTKVEIKRTVAAPSVF.I’ (SEQ ID NO: 296) peptide might appear in multiple forms such as ‘L.AFGGGTKVEIKRTVAAPSVE.I’ (SEQ ID NO: 297) and ‘F.GGGTKVEIKR’ (SEQ ID NO: 298). The non-specific cleavages greatly increase the complexity of the sample for analysis, in both peptide mapping and proteomic applications. The graph at the right shows relative abundance of the specific and non-specific cleavages.

Prominent autoproteolysis (FIG. 3): The figure shows autoproteolytically cleaved chymotryptic peptides induced by chymotrypsin self-cleavage (autoproteolysis). The autoproteolytic peptides interfere with the identification and characterization of the peptides of interest generated from an analyzed protein. Moreover, autoproteolysis decreases the enzyme's activity, leading to partial or inconsistent digestion, further complicating the analytical process.

Establishing recombinant chymotrypsin production in yeast Pichia pastoris. Since native chymotrypsins use a specialized system for activation that is absent in Pichia, novel methods for activation that can be used with recombinant chymotrypsins produced by Pichia were invented. First, the production of a family of recombinant chymotrypsins in yeast Pichia pastoris was established. Next, methods for activation of recombinant chymotrypsins were established.

Use of the yeast Pichia pastoris for recombinant chymotrypsin production was an important embodiment, with another embodiment being the development of recombinant chymotrypsins with improved properties compared to the commercially available native chymotrypsin. The goal was achieved using two consecutive approaches. First, a library of recombinant chymotrypsins from different organisms was screened with the goal of identifying the enzyme with improved properties. The identified chymotrypsin was further improved using targeted amino acid substitutions.

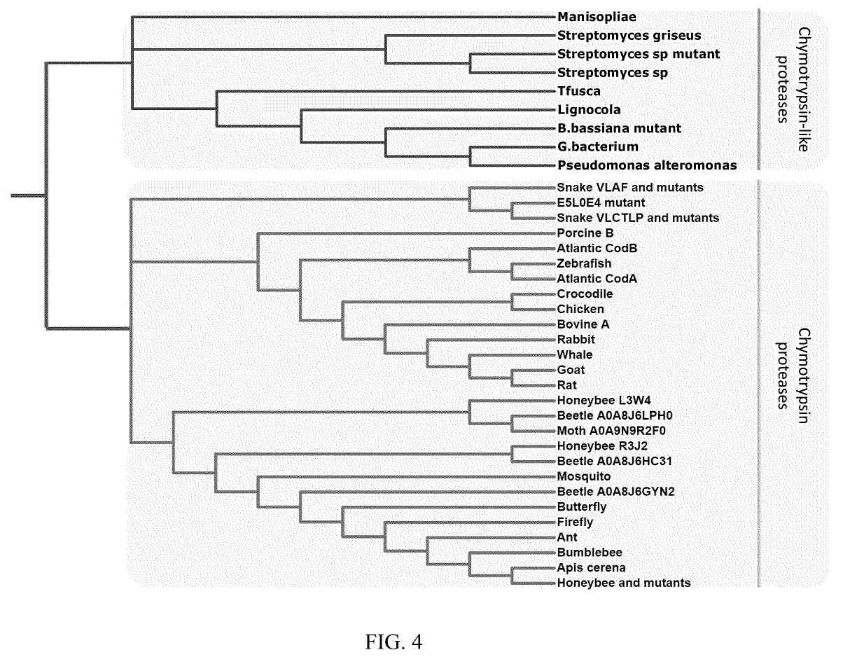

Bioinformatics tools were used to identify the enzymes that were expected to have chymotrypsin properties, from different organisms. Genomes of the screened organisms were subjected to homology search based on use of bovine chymotrypsin sequence. This provided the candidate library shown in FIG. 4. The library included chymotrypsins or chymotryptic-like proteases from mammals, birds, insects, fungi, aquatic species, reptiles (snakes), and bacteria, as depicted in FIG. 4. The library also included various mutants from several chymotrypsins, including various honeybee chymotrypsin mutants.

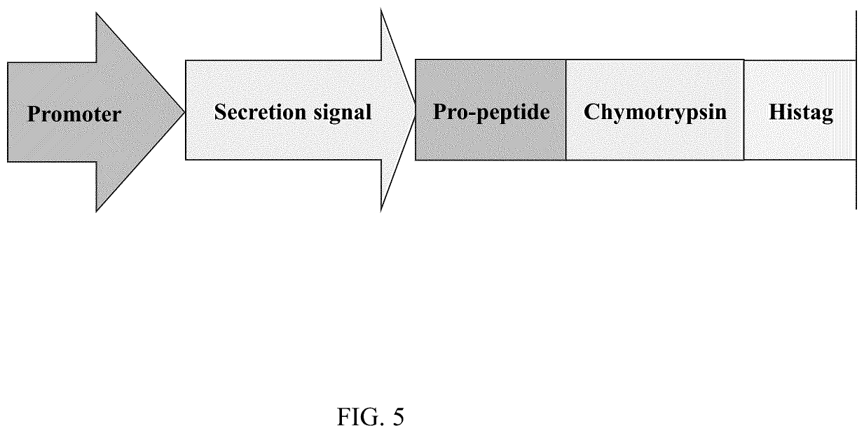

For optimal expression in Pichia pastoris, the coding gene sequences were engineered with known efficient secretion signal sequences and specialized promoters (FIG. 5). Genomic integration of the gene constructs into Pichia pastoris ensured stable expression and circumvents the problems associated with use of plasmid-based gene expression constructs. The transformed strains, harboring the integrated chymotrypsin genes, were cultivated in media that promoted optimal protease expression. Upon induction, the chymotrypsins were secreted in a soluble form into the culture medium, streamlining downstream applications.

The secreted recombinant chymotrypsins were harvested from Pichia pastoris media, with initial steps including centrifugation or filtration to remove cells and cell debris. The recombinant chymotrypsins were then purified using ion exchange chromatography, allowing efficient separation from other proteins and impurities. Additionally, an optional step included using a C-terminal histidine tag engineered into the chymotrypsins sequences, enabling purification with nickel-nitrilotriacetic acid (Ni-NTA) or similar systems, to further enhance purity.

The study showed that successful expression of recombinant chymotrypsins in Pichia is not warranted. Indeed, Atlantic cod chymotrypsin B, S. griseus chymotrypsin were not expressed in Pichia (Table 2). Snake chymotrypsins VLCTLP, VLAF, and their variants, porcine chymotrypsin B variant N127Q, B. bassani N81L, and a few selected honeybee chymotrypsin mutants showed trace-level expression detected by western blot but fail to express in sufficient amounts for characterization (Table 2).

TABLE 2
Chymotrypsin and chymotrypsin-like proteases that
were tested for expression in Pichia pastoris
Table 2: Chymotrypsin and chymotrypsin-like proteases
that were tested for expression in Pichia pastoris

| Chymotrypsin name | Expression in Pichia pastoris |
|---|---|
| Bovine (Bos taurus) chymotrypsin | yes |
| Porcine (Sus scrofa) chymotrypsin B | yes |
| Porcine (Sus scrofa) chymotrypsin variant N127Q | poor expression |
| Cod (Gadus morhua) chymotrypsin A | yes |
| Cod (Gadus morhua) chymotrypsin B | no expression |
| Zebrafish (Danio rerio) chymotrypsin | yes |
| Beauveria bassiana chymotrypsin variant S81L | poor expression |
| Snake (Daboia siamensis) chymotrypsin E5L0E4 variant | yes |
| T62R/N81S/N83S/N134Q | |
| Snake (Macrovipera lebetinus) chymotrypsin VLCTLP | poor expression |
| Snake (Macrovipera lebetinus) chymotrypsin VLCTLP variant | poor expression |
| N26Q/N82Q/N98Q/N135Q/N232Q | |
| Snake (Macrovipera lebetinus) chymotrypsin VLCTLP variant | no expression |
| N26Q/N82Q/N98Q/N135Q/N232A | |
| Snake (Macrovipera lebetinus) chymotrypsin VLAF | poor expression |
| Snake (Macrovipera lebetinus) chymotrypsin VLAF variant | no expression |
| N26Q/N61Q/N83Q | |
| Goat (Capra hircus) chymotrypsin | yes |
| Rat (Rattus norvegicus) chymotrypsin | yes |
| Rabbit (Oryctolagus cuniculus) chymotrypsin | yes |
| Whale (Balaenoptera acutorostrata scammoni) chymotrypsin | yes |
| Chicken (Gallus gallus) chymotrypsin | yes |
| Crocodile (Crocodylus porosus) chymotrypsin | yes |
| Bumble bee (Bombus vosnesenskii) chymotrypsin | yes |
| Asian honeybee (Apis cerena cerena) chymotrypsin | yes |
| Ant (Solenopsis invicta) chymotrypsin | poor expression |
| Butterfly (Danaus plexippus Plexippus) chymotrypsin | poor expression |
| Firefly (Photinus pyralis) chymotrypsin | no expression |
| Mosquito (Aedes aegypti) chymotrypsin | no expression |
| Beetle (Tenebrio molitor) chymotrypsin A0A8J6LPH0 | yes |
| Beetle (Tenebrio molitor) chymotrypsin A0A8J6HC31 | yes |
| Beetle (Tenebrio molitor) chymotrypsin A0A8J6GYN2 | yes |
| Moth (Diatraea saccharalis) chymotrypsin A0A9N9R2F0 | poor expression |
| Honeybee (Apis mellifera) chymotrypsin L3W4 | yes |
| Honeybee (Apis mellifera) chymotrypsin R3J2 | yes |
| Honeybee (Apis mellifera) chymotrypsin | yes |
| Honeybee (Apis mellifera) chymotrypsin variant | yes |
| A180S/V191C/P204DI/D211V | |
| Honeybee (Apis mellifera) chymotrypsin variant C127A | yes |
| Honeybee (Apis mellifera) chymotrypsin variant | yes |
| C127A/I128C/V190C | |
| Honeybee (Apis mellifera) chymotrypsin variant | yes |
| K29G/Q62V/R84P/V191C | |
| Honeybee (Apis mellifera) chymotrypsin variant | yes |
| N28Q/K29G/Q62V/R84P/V191C | |
| Honeybee (Apis mellifera) chymotrypsin variant A180P | yes |
| Honeybee (Apis mellifera) chymotrypsin variant A180V | yes |
| Honeybee (Apis mellifera) chymotrypsin variant D193GP | yes |
| Honeybee (Apis mellifera) chymotrypsin variant D211G | yes |
| Honeybee (Apis mellifera) chymotrypsin variant D211H | yes |
| Honeybee (Apis mellifera) chymotrypsin variant E153I | yes |
| Honeybee (Apis mellifera) chymotrypsin variant F173L | yes |
| Honeybee (Apis mellifera) chymotrypsin variant F201W | yes |
| Honeybee (Apis mellifera) chymotrypsin variant | yes |
| F229A/L230N/Δ231-245 | |
| Honeybee (Apis mellifera) chymotrypsin variant F242A/Y243R | yes |
| Honeybee (Apis mellifera) chymotrypsin variant G177I | yes |
| Honeybee (Apis mellifera) chymotrypsin variant | yes |
| G179S/A180S/V191C/P204ST/D211V | |
| Honeybee (Apis mellifera) chymotrypsin variant K7Y | no expression |
| Honeybee (Apis mellifera) chymotrypsin variant | poor expression |
| K7Y/F242A/Y243R | |
| Honeybee (Apis mellifera) chymotrypsin variant | no expression |
| K7Y/S228A/F229N/Δ231-245 | |
| Honeybee (Apis mellifera) chymotrypsin variant K7Y/V191C | no expression |
| Honeybee (Apis mellifera) chymotrypsin variant | poor expression |
| K7Y/V191C/Δ229-245 | |
| Honeybee (Apis mellifera) chymotrypsin variant N28Q | poor expression |
| Honeybee (Apis mellifera) chymotrypsin variant P204A | yes |
| Honeybee (Apis mellifera) chymotrypsin variant P204C/C205P | yes |
| Honeybee (Apis mellifera) chymotrypsin variant V191C | yes |
| Honeybee (Apis mellifera) chymotrypsin variant V191C/Δ229- | yes |
| 245 | |
| Honeybee (Apis mellifera) chymotrypsin variant V199G | yes |
| Honeybee (Apis mellifera) chymotrypsin variant V199W | yes |
| Honeybee (Apis mellifera) chymotrypsin variant Δ229-245 | yes |
| Gammaproteobacteria bacterium chymotrypsin-like protease | yes |
| Pseudoalteromonas sp. SM1988 chymotrypsin-like protease | yes |
| Scytalidoglutamic (Scytalidium lignicola) peptidase | yes |
| Metarhizium anisopliae chymotrypsinogen-like protease | yes |
| T. fusca chymotrypsinogen-like protease | yes |
| Streptomyces griseus Protease C (chymotrypsin-like protease) | no expression |
| Streptomyces sp. chymotrypsinogen-like protease | no expression |
| Streptomyces sp. chymotrypsinogen-like protease propeptide | no expression |
| variant | |

Activation of Recombinant Chymotrypsins. Chymotrypsins are expressed in the form of zymogen. The zymogen is the inactive protease form. It requires cleavage of the propeptide at a maturation site to become activated. The following methods were developed for activation of the recombinant chymotrypsin zymogens expressed in Pichia: trypsin-facilitated activation, self-activation, and activation with exoproteases (aminopeptidases). Sequencing of the recombinant chymotrypsin N-terminus with Edman sequencing was used to confirm cleavage of the propeptide and map the maturation site (Table 3).

TABLE 3
Origin, Maturation Methods, and N-Terminus Analysis of Recombinant
Chymotrypsins

| Origin source | | Experimentally | Protease | |
|---|---|---|---|---|
| for recombinant | Authentic | determined | maturation | |
| Chymotrypsin | N-terminal | N-terminal | method | Activity |
| Porcine | CGVPA | Not determined | Self-activation/ | Active |
| polypeptide 1 | IVNGE | | inculture | |
| polypeptide 2 | | | | |
| Honeybee | IVGGK | ^{∧∧}GGKNA | Self-maturation/ | Active |
| | | | inculture | |
| Honeybee | IVGGK | IVGGKNA | Self-maturation/ | Active |
| | | | inculture | |
| Porcine | CGVPA | Not determined | Trypsin pre- | Active |
| polypeptide 1 | IVNGE | | incubation | |
| polypeptide 2 | | | | |
| Bovine | CGVPA | Not determined | Trypsin pre- | Active |
| polypeptide 1 | IVNGE | IVNGE | incubation | |
| polypeptide 2 | ANTPD | Not determined | | |
| polypeptide 3 | | | | |
| Bovine | CGVPA | Not determined | Self-maturation/ | Low activity, |
| polypeptide 1 | IVNGE | RIVNG | inculture | became fully |
| polypeptide 2 | ANTPD | ANTPD | | active after |
| polypeptide 3 | | | | treatment with |
| | | | | aminopeptidase |

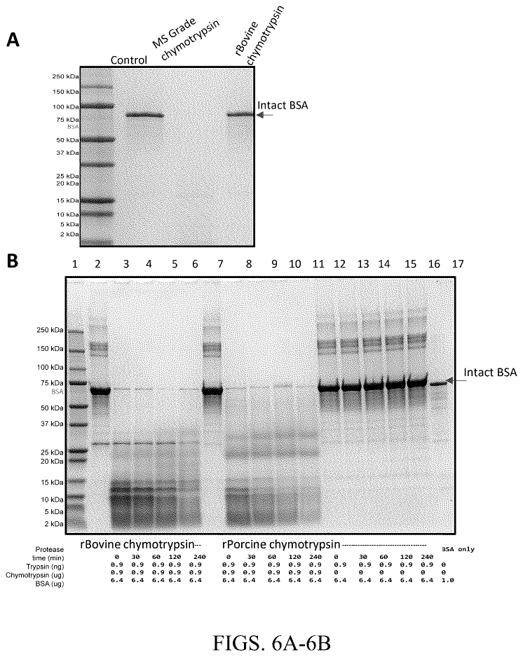

The trypsin-facilitated activation method mimics chymotrypsin activation in animal pancreas. In pancreas, trypsin specifically cleaves at the R/K pro-peptide site on the chymotrypsin zymogen, facilitating its conversion to an active form. Trypsin-facilitated activation proved to be effective in activation of bovine, porcine, zebrafish, and Cod A recombinant chymotrypsins in the study. In more detail, inactive recombinant chymotrypsin zymogens were incubated with trypsin. The incubation time with trypsin was minimized to prevent degradation of the recombinant chymotrypsin. To validate the efficacy of this activation method, the chymotrypsins were tested for proteolytic activity by incubating with bovine serum albumin (BSA) and monitoring for BSA degradation (FIG. 6).

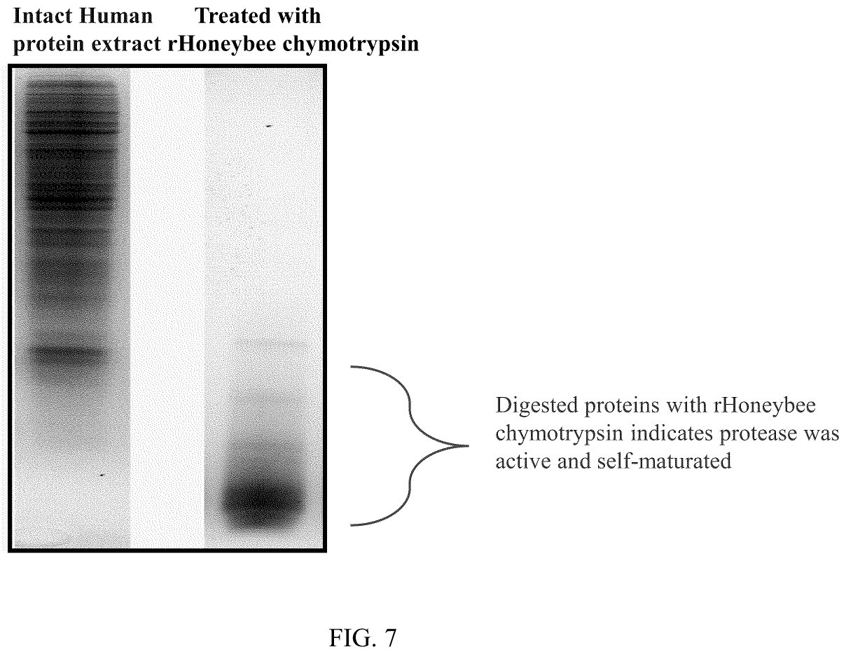

In the self-activation method, recombinant chymotrypsin zymogens were incubated in the culture supernatant for an extended period. Certain recombinant chymotrypsin zymogens self-activated at these conditions. Edman sequencing confirmed that the self-activation was conducted via removing of pro-peptide (Table 3), thus converting an inactive zymogen into an active chymotrypsin. This circumvents the need for trypsin or other activating proteases, thereby streamlining the manufacturing process. The activation was confirmed by the digestion of a human protein extract with the self-activated, recombinant chymotrypsins (FIG. 7; “rHoneybee chymotrypsin” indicates the recombinant honeybee chymotrypsin). This method was effective in activation of porcine and honeybee recombinant chymotrypsins, as well as T. fusca, M. anisopliae, and G. bacterium chymotrypsin-like proteases (Table 2). Additionally, Pichia pastoris possesses highly active and efficient proteases. It is likely that these host proteases play a significant role in the maturation and activation of several of these recombinant chymotrypsin zymogens, thereby enhancing the efficiency of the production process.

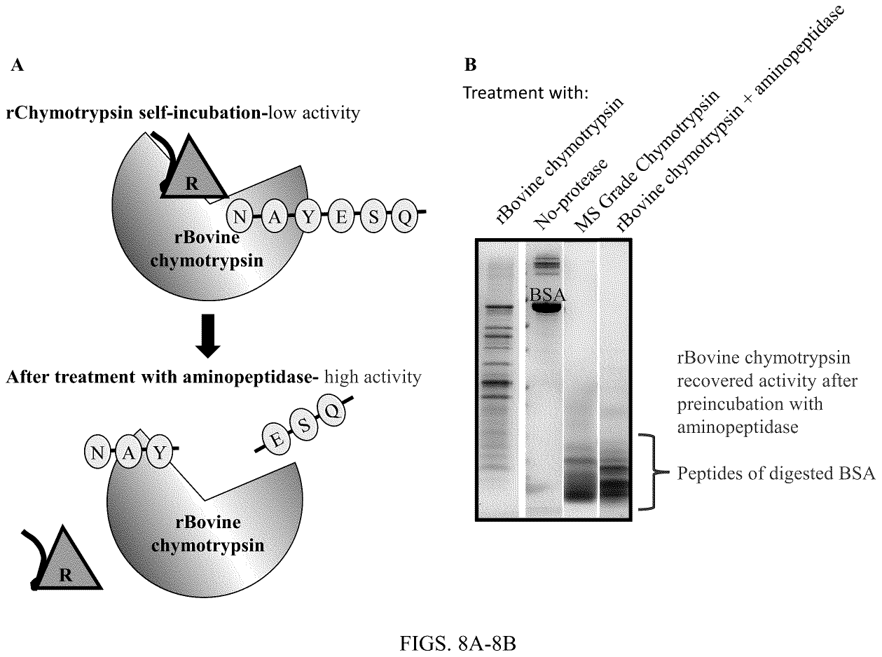

The exoprotease (aminopeptidase)-facilitated activation uses the combination of two cleavage steps: partial self-cleavage of pro-peptide (partial self-maturation) followed by extra degradation of a remaining pro-peptide sequence with an exoprotease (aminopeptidase). The method successfully activated recombinant bovine chymotrypsin. The recombinant chymotrypsin zymogen was incubated in the culture supernatant for an extended time to enable partial self-maturation (FIG. 8, Panel A; see N-terminus of the incompletely maturate bovine chymotrypsin in Table 3). This step resulted in relatively low proteolytic activity indicating incomplete enzyme activation (see testing results in FIG. 8, Panel B). Efforts to remove the extra arginine and restore full enzymatic activity with trypsin were unsuccessful. To achieve appropriate enzyme activation, the enzyme was subjected to treatment with an exoprotease, aminopeptidase, which removed the remaining pro-peptide sequence and enabled appropriate enzyme activation (FIG. 8, Panel B).

Table 3 shows the experimental data supporting specific pro-peptide processing for recombinant bovine chymotrypsin, recombinant porcine chymotrypsin, and recombinant honeybee chymotrypsin. The table also describes the methods used to activate those recombinant chymotrypsins.

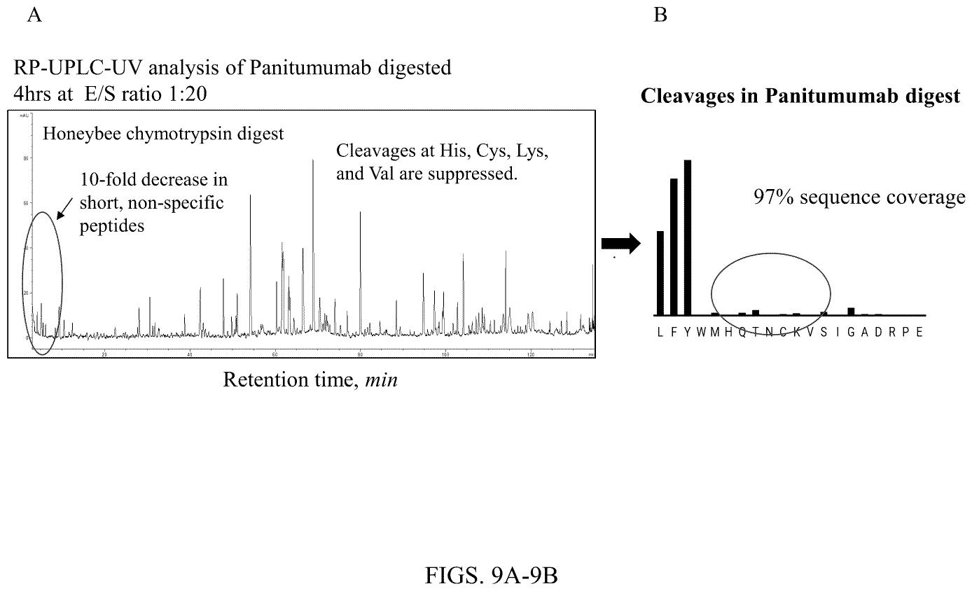

Improved cleavage specificity of the recombinant honeybee chymotrypsin. It was discovered that the recombinant honeybee chymotrypsin has improved cleavage specificity compared to the commercially available bovine chymotrypsin. Peptide mapping of a model antibody Panitumumab using the recombinant honeybee chymotrypsin showed that the non-specific cleavages at methionine, histidine, threonine, and valine, commonly observed in the digests with bovine chymotrypsin, were suppressed (FIG. 9, compared to FIG. 2). Cleavages at leucine residues were still observed, but they were induced at a reduced level compared to the digests with bovine chymotrypsin. The additional advantage of honeybee chymotrypsin is its significantly reduced cleavage after tryptophan (W), which is beneficial for the biotherapeutic protein analysis (e.g., characterization of tryptophan modifications).

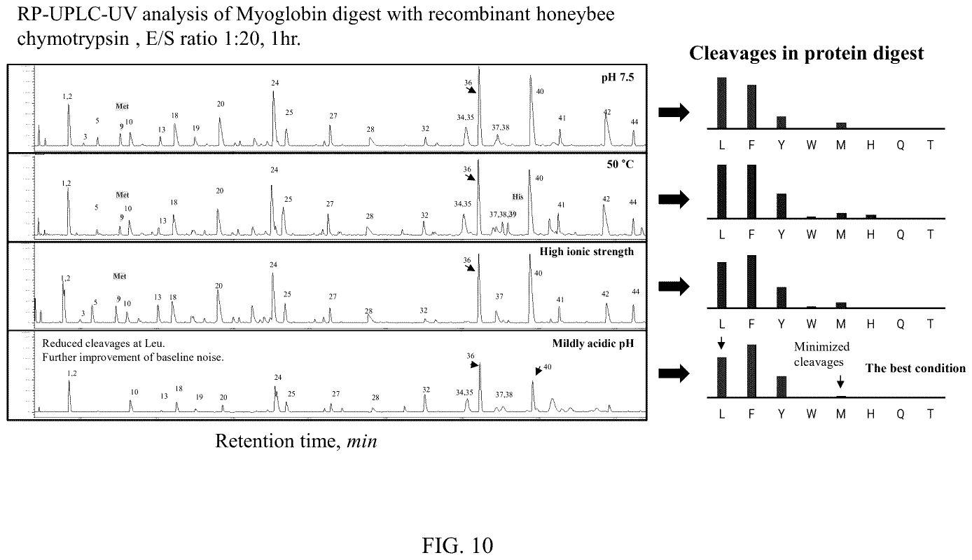

An additional discovery was made. The enzyme cleavage specificity was further increased by conducting protein digestion with the recombinant honeybee chymotrypsin under non-conventional, mildly acidic conditions. Protein digestion with chymotrypsin is conventionally performed at alkaline pH as the bovine chymotrypsin has the highest activity at these conditions. Unexpectedly, it was found that the recombinant honeybee chymotrypsin retained sufficiently high activity at mildly acidic pH and, most importantly, its cleavage specificity was further improved. Specifically, non-specific cleavages were virtually eliminated at mildly acidic conditions (FIG. 10, compare top and bottom chromatograms).

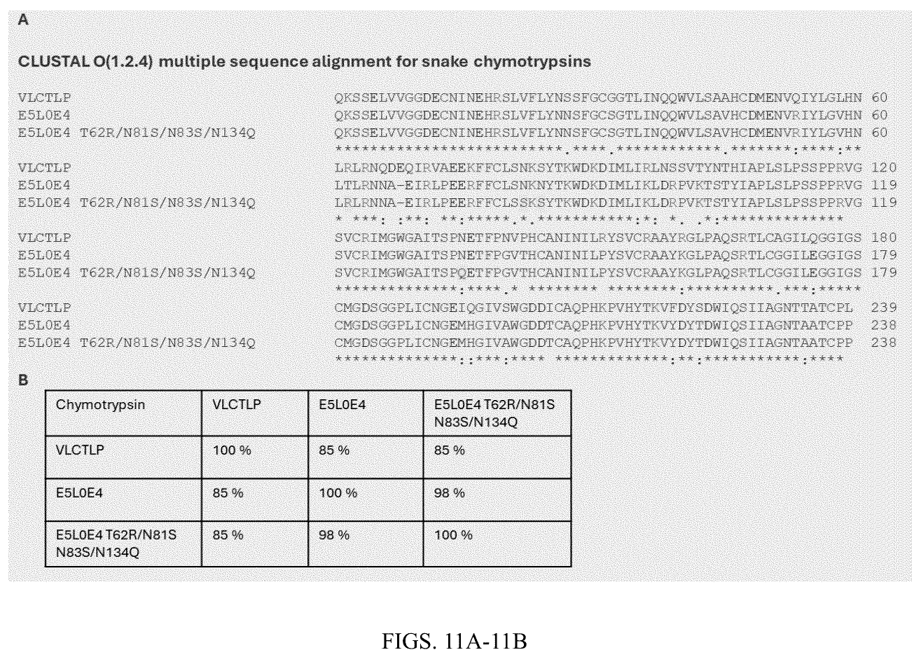

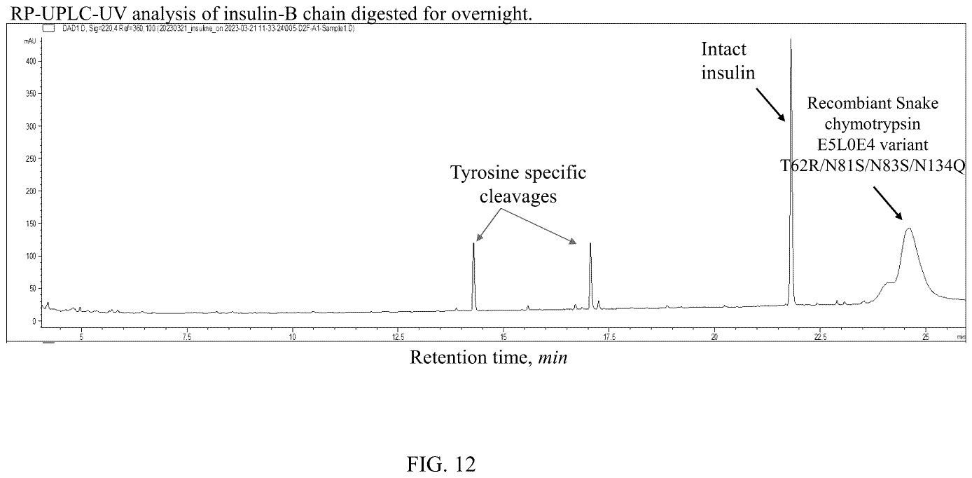

Unique, tyrosine-specific cleavage activity of the recombinant snake protease. The unique, tyrosine-specific cleavage activity of the snake chymotrypsin has been reported for the native snake chymotrypsin purified from the venom of the snake Macrovipera lebetina (VI.CTLP). Attempts in this study to express and activate VLCTLP and its variants in Pichia pastoris failed. This failure led to the exploration of close homologues through bioinformatics, which identified the E5L0E4 chymotrypsin from the snake Daboia siamensis (FIG. 11A). E5L0E4 shares approximately 85% sequence identity with VICTLP (FIG. 11B). The sequence was strategically modified to enhance expression and purification. The removal of potential N-glycosylation sites recognized by Pichia pastoris as NXS/T sites was targeted as glycosylation can interfere with the expression and purification of recombinant proteins. These modification sites in the E5L0E4 sequence are highlighted in FIG. 11A. The approach involved substituting glycosylation sites with QXS/T, combined with a rational design based on amino acid sequence homology to other chymotrypsins that lacked those potential glycosylation sites. This strategic modification resulted in the E5L0E4 variant exhibiting significantly improved expression levels for its recombinant form in Pichia pastoris. The cleavage specificity of this enzyme was investigated using insulin beta chain as a model substrate and the digested products were analyzed with LC-UV (FIG. 12). The data indicated that the recombinant T62R/N81S/N83S/N134Q mutant of the snake Daboia siamensis chymotrypsin reproduced the unique, tyrosine cleavage specificity observed in native chymotrypsin from Macrovipera lebetina. A protease with tyrosine cleavage specificity is a highly valuable tool that would significantly boost protein analysis, including mass spectrometry and other analytical techniques, as well as facilitate the preparation of digested proteins for various applications.

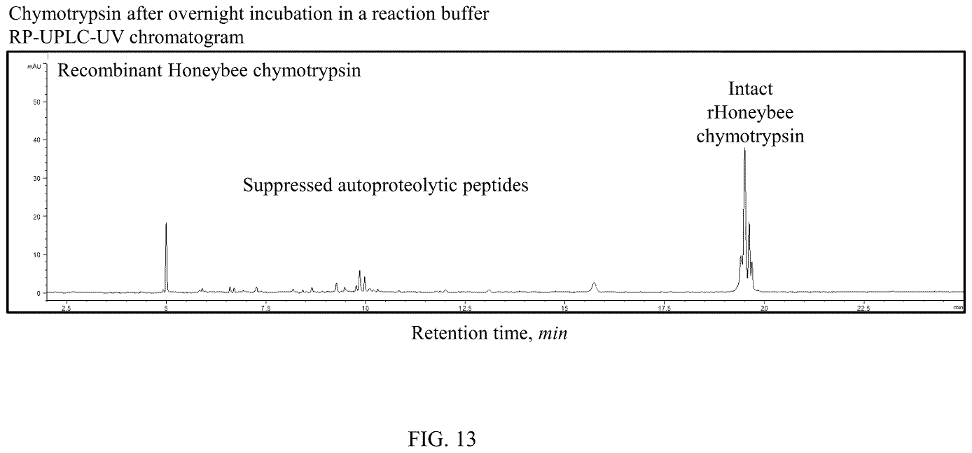

Improved autoproteolytic resistance of the recombinant honeybee chymotrypsin. The recombinant honeybee chymotrypsin demonstrated an advantage, that of high autoproteolytic resistance. The native bovine chymotrypsin currently used in protein analysis is prone to prominent autoproteolysis. Autoproteolytic peptides compromise the analysis of peptides of interest during peptide mapping and proteomic applications. Autoproteolysis also decreases chymotrypsin digestion efficiency. It was discovered that the recombinant honeybee chymotrypsin had significantly increased autoproteolytic resistance further increasing the value of this enzyme for protein analysis (FIG. 13).

Mutants of the recombinant honeybee chymotrypsin with increased enzyme activity and thermostability. Recombinant enzymes often demonstrate lower activity than their recombinant analogs and the recombinant chymotrypsins are no exception. Nevertheless, mutagenesis was used to try to develop recombinant honeybee chymotrypsin with increased activity and stability.

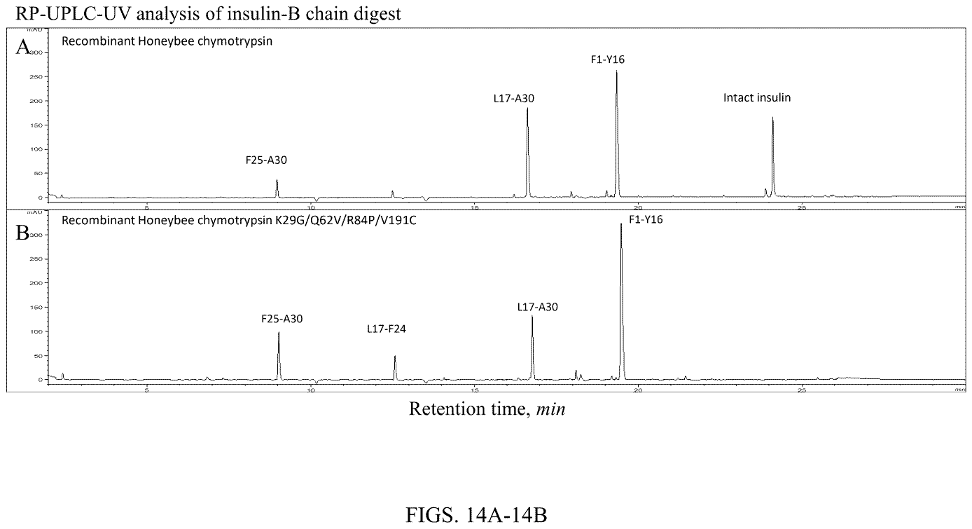

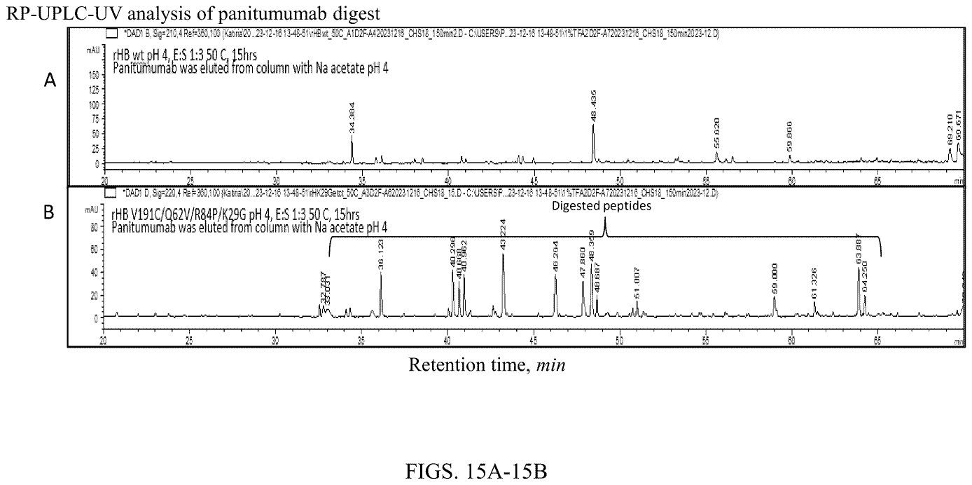

The following amino acid mutations were introduced: K29G, Q62V, R84P, and V191C. The mutant showed higher activity than the wild-type form (FIG. 14). The increased activity was observed at elevated temperatures (50° C.) and acidic pH (FIG. 15). This data indicated that the mutations increased both activities and thermostability of the recombinant honeybee chymotrypsin.

Mutants of the recombinant honeybee chymotrypsin with modified cleavage specificity. Mutagenesis was also used to modify cleavage specificity of the recombinant honeybee chymotrypsin. To achieve this goal, a library of 32 recombinant honeybee chymotrypsin variants was created. 24 mutants were successfully expressed and self-activated. Upon characterization, 10) mutants with modified cleavage specificity were found. In more detail, these mutants cleaved leucine and alanine residues.

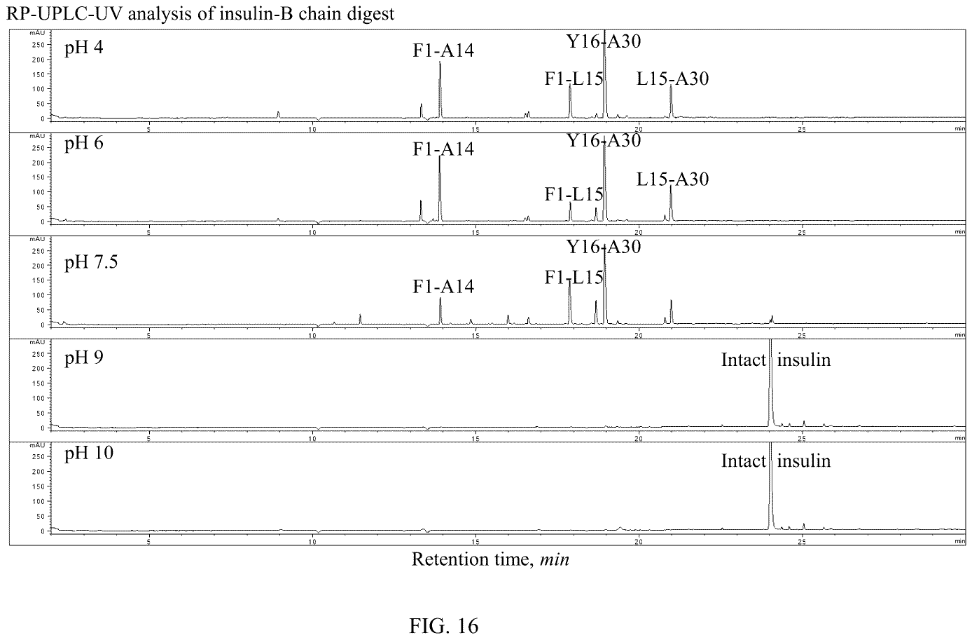

Recombinant honeybee chymotrypsin variants with mutations at the D211 position exhibited altered cleavage specificity preferences, primarily cleaving after leucine or alanine (L/A) under lower pH conditions. Specifically, the variants (1) D211G, (2) D211H, (3) A180S/V191C/P204DI/D211V, and (4) G179S/A180S/V191C/P204ST/D211V demonstrated these characteristics. The A180S/V191C/P204DI/D211V and G179S/A180S/V191C/P204ST/D211V variants were found to be catalytically inactive at pH levels of 9 and 10 (FIG. 16). However, they retained their ability to cleave after L/A when the pH was lowered to below 7.5.

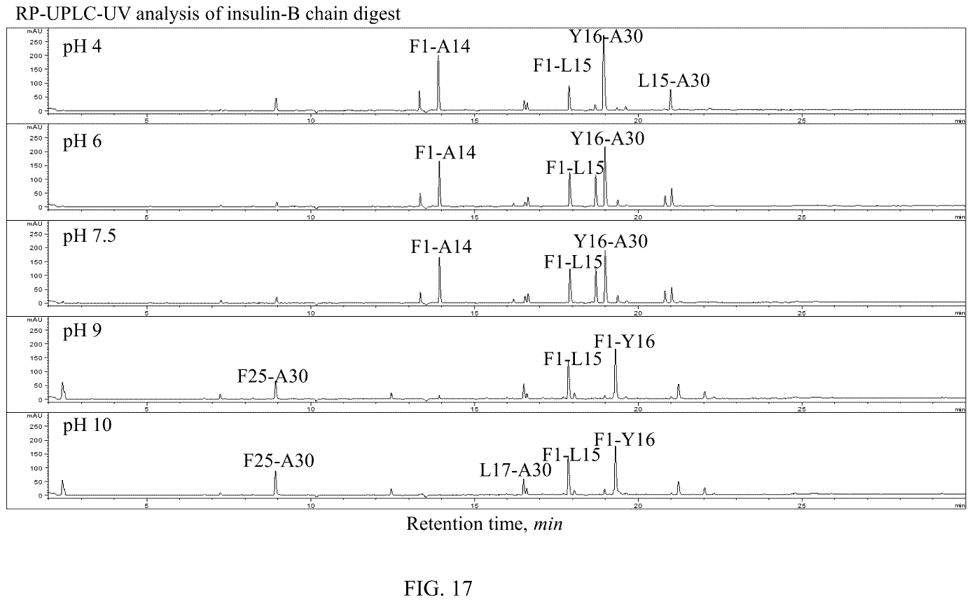

In contrast, the D211G and D211H variants maintained their cleavage activity across a broader range of pH conditions, up to pH 10, albeit with a shift in their specificity (FIG. 17). Under acidic conditions, these variants predominantly cleaved after L/A. As the pH increased, a change in the cleavage specificities were observed; with increased catalytic activity against Y/F. At pH 10 cleavages were predominantly after Y/F/L but not alanine.

Comparatively, mutations at the equivalent site in rat yielded a different outcome. For instance, in rat chymotrypsin B, the aspartic acid (D211) in honeybee chymotrypsin is an alanine (A226), and in bovine chymotrypsin, it is a glycine (G). Experimental investigations into an alanine to glycine (A226G) mutation in rat chymotrypsin revealed a new cleavage specificity towards tryptophan and leucine, with the primary cleavage sites being tyrosine, phenylalanine, and tryptophan (Y/F/W). However, the honeybee chymotrypsin variants D211G and D211H did not show primarily cleavage preference for tyrosine or phenylalanine. An additional example arises from nature, porcine chymotrypsin B has a glycine at this equivalent position. However, other data has shown that it does not cleave after tryptophan. Thus, while mutations at the D211 site in honeybee chymotrypsin were anticipated to modify cleavage specificity, the exact effects observed in this investigation were unexpected.

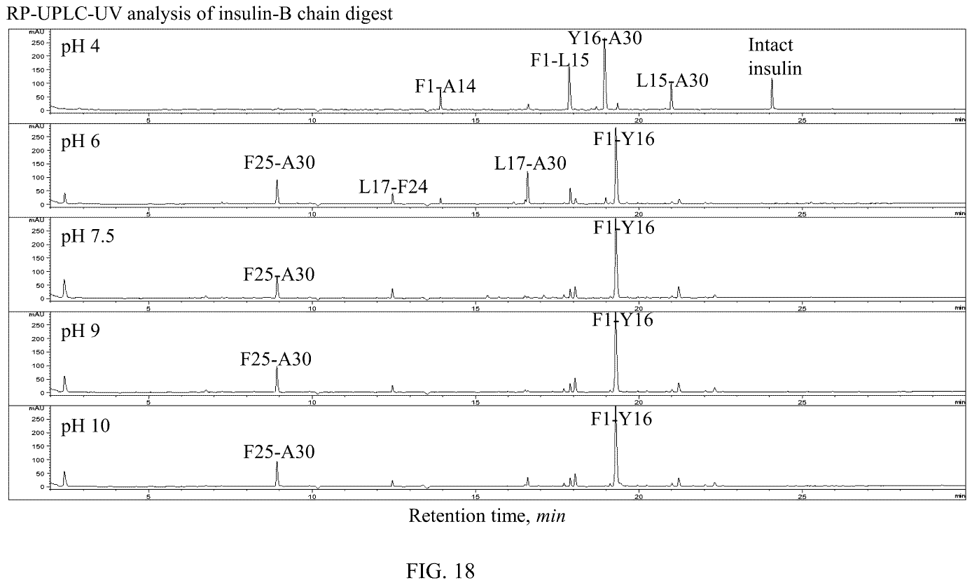

The specific variants of recombinant honeybee chymotrypsin that were identified are as follows: (1) V199G, (2) E153I, (3) F173L, (4) F229N/L230/ Δ 231-245, (5) C127A/I128C/V190C, and (6) F242A/Y243R. Under conditions of neutral pH, these variants demonstrated cleavage activities that align closely with those of the wildtype recombinant honeybee chymotrypsin. Under reactions conditions at pH 4, these variants exhibited a unique and previously undocumented cleavage specificity towards L/A (FIG. 18). The observed modulation of cleavage specificity towards L/A under low pH conditions in these recombinant honeybee chymotrypsins were unforeseen and novel.

What is claimed is:

1-144. (canceled)

145. A composition comprising:
(a) a cell comprising an exogenous nucleic acid sequence encoding a recombinant chymotrypsin polypeptide comprising at least 70% sequence identity to SEQ ID NO: 32 or 33;
(b) a nucleic acid sequence encoding a recombinant chymotrypsin polypeptide comprising at least 70% sequence identity to SEQ ID NO: 32 or 33; or
(c) a recombinant chymotrypsin polypeptide comprising at least 70% sequence identity to SEQ ID NO: 32 or 33.

146. The composition of claim 145, wherein the nucleic acid sequence is stably integrated into the genome of the cell.

147. The composition of claim 145, wherein the nucleic acid sequence is transiently expressed in the cell.

148. The composition of claim 145, wherein the cell is a yeast cell.

149. The composition of claim 148, wherein the cell is Pichia pastoris.

150. The composition of claim 145, wherein the recombinant chymotrypsin polypeptide comprises one or more of a secretion signal, a pro-peptide, or a purification tag.

151. The composition of claim 145, wherein the exogenous nucleic acid sequence further comprises a promoter and/or encodes any one or more of a secretion signal, a pro-peptide, or a purification tag.

152. The composition of claim 151, wherein the purification tag is a C-terminal histidine tag.

153. The composition of claim 145, wherein the exogenous nucleic acid sequence comprises at least 70% sequence identity to SEQ ID NO: 105 or 106.

154. The composition of claim 145, wherein the mutant honeybee recombinant chymotrypsin comprises one or more of a S at position 180, a C at position 191, a DI at position 204, and a V at position 211 relative to SEQ ID NO: 34.

155. The composition of claim 145, wherein the recombinant chymotrypsin polypeptide further comprises at least 70% sequence identity to SEQ ID NO: 20.

156. The composition of claim 145, wherein the recombinant chymotrypsin polypeptide further comprises at least 70% sequence identity to SEQ ID NO: 21.

157. The composition of claim 145, wherein the recombinant chymotrypsin polypeptide comprises at least 90% sequence identity to SEQ ID NO 32 or 33.

158. The composition of claim 157, wherein the recombinant chymotrypsin polypeptide comprises 100% sequence identity to SEQ ID NO 32 or 33.

159. The composition of claim 145, wherein the recombinant chymotrypsin polypeptide comprises 100% sequence identity to SEQ ID NO: 20.

160. The composition of claim 145, wherein the recombinant chymotrypsin polypeptide comprises at least 100% sequence identity to SEQ ID NO: 21.
